(19) United States
(12) Patent Application Publication
(10) Pub. No.: US 2011/0023143 A1
(43) Pub. Date: Jan. 27, 2011

(54) GENOMIC EDITING OF NEURODEVELOPMENTAL GENES IN ANIMALS

(75) Inventors: Edward Weinstein, St. Louis, MO (US); Xiaoxia Cui, St. Louis, MO (US); Phil Simmons, St. Louis, MO (US)

Correspondence Address: POLSINELLI SHUGHART PC, 700 W. 47TH STREET, SUITE 1000, KANSAS CITY, MO, 64112-1802, US

(73) Assignee: SIGMA-ALDRICH CO., St. Louis, MO (US)

(21) Appl. No.: 12/842,578

(22) Filed: Jul. 23, 2010

Related U.S. Application Data
(63) Continuation-in-part of application No. 12/592,852, filed on Dec. 3, 2009.
(60) Provisional application No. 61/343,287, filed on Apr. 26, 2010.
(60) Provisional application No. 61/323,702, filed on Apr. 13, 2010.
(60) Provisional application No. 61/323,719, filed on Apr. 13, 2010.
(60) Provisional application No. 61/323,698, filed on Apr. 13, 2010.
(60) Provisional application No. 61/309,729, filed on Mar. 2, 2010.
(60) Provisional application No. 61/308,089, filed on Feb. 25, 2010.
(60) Provisional application No. 61/336,000, filed on Jan. 14, 2010.
(60) Provisional application No. 61/263,904, filed on Nov. 24, 2009.
(60) Provisional application No. 61/263,696, filed on Nov. 23, 2009.
(60) Provisional application No. 61/245,877, filed on Sep. 25, 2009.
(60) Provisional application No. 61/232,620, filed on Aug. 10, 2009.
(60) Provisional application No. 61/228,419, filed on Jul. 24, 2009.
(60) Provisional application No. 61/200,985, filed on Dec. 4, 2008.
(60) Provisional application No. 61/205,970, filed on Jan. 26, 2009.

(51) Int. Cl. G01N 33/00; A01K 67/00; C12N 5/10

(52) U.S. Cl. 800/3; 800/13; 800/15; 800/16; 800/17; 800/14; 435/325; 435/351; 435/350; 435/366; 435/363; 435/352; 435/353

(57) ABSTRACT

The present invention provides genetically modified animals and cells comprising edited chromosomal sequences encoding proteins that are associated with neurodevelopmental disorders. In particular, the animals or cells are generated using a zinc finger nuclease-mediated editing process. Also provided are methods of using the genetically modified animals or cells disclosed herein to screen agents for toxicity and other effects.

### CROSS-REFERENCE TO RELATED APPLICATIONS

This application claims the priority of U.S. provisional application No. 61/343,287, filed Apr. 26, 2010, U.S. provisional application No. 61/323,702, filed Apr. 13, 2010, U.S. provisional application No. 61/323,719, filed Apr. 13, 2010, U.S. provisional application No. 61/323,698, filed Apr. 13, 2010, U.S. provisional application No. 61/309,729, filed Mar. 2, 2010, U.S. provisional application No. 61/308,089, filed Feb. 25, 2010, U.S. provisional application No. 61/336,000, filed Jan. 14, 2010, U.S. provisional application No. 61/263,904, filed Nov. 24, 2009, U.S. provisional application No. 61/263,696, filed Nov. 23, 2009, U.S. provisional application No. 61/245,877, filed Sep. 25, 2009, U.S. provisional application No. 61/232,620, filed Aug. 10, 2009, U.S. provisional application No. 61/228,419, filed Jul. 24, 2009, and is a continuation in part of U.S. non-provisional application Ser. No. 12/592,852, filed Dec. 3, 2009, which claims priority to U.S. provisional 61/200,985, filed Dec. 4, 2008 and U.S. provisional application 61/205,970, filed Jan. 26, 2009, all of which are hereby incorporated by reference in their entirety.

### FIELD OF THE INVENTION

The invention generally relates to genetically modified animals or cells comprising at least one edited chromosomal sequence encoding a neurodevelopmental protein. In particular, the invention relates to the use of a zinc finger nuclease-mediated process to edit chromosomal sequences encoding neurodevelopmental proteins in animals or cells.

### BACKGROUND OF THE INVENTION

A number of genes have been associated with complex neurodevelopmental disorders, which may adversely impact cognitive and social functions in affected individuals. The progress of ongoing research into the causes and treatments of these neurodevelopmental disorders is hampered by the onerous task of developing an animal model, which incorporates the genes proposed to be involved in the development or severity of the neurodevelopmental disorders.

Conventional methods such as gene knockout technology may be used to edit a particular gene in a potential model organism in order to develop an animal model of a neurodevelopmental disorder. However, gene knockout technology may require months or years to construct and validate the proper knockout models. In addition, genetic editing via gene knockout technology has been reliably developed in only a limited number of organisms, such as mice. Even in a best case scenario, mice typically show low intelligence, making mice a poor choice of organism in which to study complex disorders of cognition and behavior. Ideally, the selection of an organism in which to model a complex neurodevelopmental disorder should be based on the organism's ability to exhibit the characteristics of the disorder as well as its amenability to existing research methods.

The rat is emerging as a genetically malleable, preferred model organism for the study of neurodevelopmental disorders, particularly because these disorders are not well-modeled in mice. Rats are a superior choice compared to mice as model organisms for the study of human diseases of cognition such as learning and memory due to their higher intelligence, complex behavioral repertoire, and observable responses to behavior-modulating drugs, all of which better approximate the human condition. Further, the larger physical size of rats relative to mice facilitates experimentation that requires dissection, in vivo imaging, or isolation of specific cells or organ structures for cellular or molecular studies of these neurodevelopmental diseases.

A need exists for animals with modification to one or more genes associated with human neurodevelopmental disorders to be used as model organisms in which to study these disorders. The genetic modifications may include gene knockouts including tissue-specific or temporal-specific knockouts using loxP-flanked (“floxed”) alleles in combination with an inducible Cre-recombinase, as well as under-expression, modified expression, or over-expression of alleles that either cause or are associated with neurodevelopmental diseases in humans. Further, a need exists for modification of one or more genes associated with human neurodevelopmental disorders in a variety of organisms in order to develop appropriate animal models of neurodevelopmental disorders.

### SUMMARY OF THE INVENTION

One aspect of the present disclosure encompasses a genetically modified animal comprising at least one edited chromosomal sequence encoding a neurodevelopmental protein.

A further aspect provides a non-human embryo comprising at least one RNA molecule encoding a zinc finger nuclease that recognizes a chromosomal sequence encoding a neurodevelopmental protein, and, optionally, at least one donor polynucleotide comprising a sequence encoding an ortholog of the neurodevelopmental protein or an edited neurodevelopmental protein.

Another aspect provides a genetically modified cell comprising at least one edited chromosomal sequence encoding a neurodevelopmental protein.

Yet another aspect provides a method for assessing the effect of an agent in a genetically modified animal. The method includes administering the agent to the genetically modified animal comprising at least one edited chromosomal sequence encoding a neurodevelopmental protein, and comparing a parameter obtained from the genetically modified animal to the parameter obtained from a wild-type animal administered the same agent. The parameter is chosen from (a) rate of elimination of the agent or its metabolite(s); (b) circulatory levels of the agent or its metabolite(s); (c) bioavailability of the agent or its metabolite(s); (d) rate of metabolism of the agent or its metabolite(s); (e) rate of clearance of the agent or its metabolite(s); (f) toxicity of the agent or its metabolite(s); and (g) ability of the agent to modify an incidence or indication of a neurodevelopmental disorder in the genetically modified animal.

Still yet another aspect encompasses a method for assessing the therapeutic potential of an agent as a treatment for a neurodevelopmental disorder. This method includes administering the agent to a genetically modified animal, wherein the genetically modified animal comprises at least one edited chromosomal sequence encoding a neurodevelopmental protein, and comparing a selected parameter obtained from the genetically modified animal to the selected parameter obtained from a wild-type animal with no exposure to the same agent. The selected parameter is chosen from a) spontaneous behaviors; b) performance during behavioral testing; c) physiological anomalies; d) abnormalities in tissues or cells; e) biochemical function; and f) molecular structures.

Other aspects and features of the disclosure are described more thoroughly below.

### DETAILED DESCRIPTION OF THE INVENTION

The present disclosure provides a genetically modified animal or animal cell comprising at least one edited chromosomal sequence encoding a neurodevelopmental protein. The edited chromosomal sequence may be (1) inactivated, (2) modified, or (3) comprise an integrated sequence. An inactivated chromosomal sequence is altered such that a functional protein is not made. Thus, a genetically modified animal comprising an inactivated chromosomal sequence may be termed a “knock out” or a “conditional knock out.” Similarly, a genetically modified animal comprising an integrated sequence may be termed a “knock in” or a “conditional knock in.” As detailed below, a knock in animal may be a humanized animal. Furthermore, a genetically modified animal comprising a modified chromosomal sequence may comprise a targeted point mutation(s) or other modification such that an altered protein product is produced. The chromosomal sequence encoding the neurodevelopmental protein generally is edited using a zinc finger nuclease-mediated process. Briefly, the process comprises introducing into an embryo or cell at least one RNA molecule encoding a targeted zinc finger nuclease and, optionally, at least one accessory polynucleotide. The method further comprises incubating the embryo or cell to allow expression of the zinc finger nuclease, wherein a double-stranded break introduced into the targeted chromosomal sequence by the zinc finger nuclease is repaired by an error-prone non-homologous end-joining DNA repair process or a homology-directed DNA repair process. The method of editing chromosomal sequences encoding a neurodevelopmental protein using targeted zinc finger nuclease technology is rapid, precise, and highly efficient.

### (I) Genetically Modified Animals

One aspect of the present disclosure provides a genetically modified animal in which at least one chromosomal sequence encoding a neurodevelopmental protein has been edited. For example, the edited chromosomal sequence may be inactivated such that the sequence is not transcribed and/or a functional neurodevelopmental protein is not produced. Alternatively, the edited chromosomal sequence may be modified such that it codes for an altered neurodevelopmental protein. For example, the chromosomal sequence may be modified such that at least one nucleotide is changed and the expressed neurodevelopmental protein comprises at least one changed amino acid residue (missense mutation). The chromosomal sequence may be modified to comprise more than one missense mutation such that more than one amino acid is changed. Additionally, the chromosomal sequence may be modified to have a three nucleotide deletion or insertion such that the expressed neurodevelopmental protein comprises a single amino acid deletion or insertion, provided such a protein is functional. The modified protein may have altered substrate specificity, altered enzyme activity, altered kinetic rates, and so forth. Furthermore, the edited chromosomal sequence may comprise an integrated sequence and/or a sequence encoding an orthologous protein associated with a neurodevelopmental disorder. The genetically modified animal disclosed herein may be heterozygous for the edited chromosomal sequence encoding a protein associated with a neurodevelopmental disorder. Alternatively, the genetically modified animal may be homozygous for the edited chromosomal sequence encoding a protein associated with a neurodevelopmental disorder.

In one embodiment, the genetically modified animal may comprise at least one inactivated chromosomal sequence encoding a neurodevelopmental protein. The inactivated chromosomal sequence may include a deletion mutation (i.e., deletion of one or more nucleotides), an insertion mutation (i.e., insertion of one or more nucleotides), or a nonsense mutation (i.e., substitution of a single nucleotide for another nucleotide such that a stop codon is introduced). As a consequence of the mutation, the targeted chromosomal sequence is inactivated and a functional neurodevelopmental protein is not produced. The inactivated chromosomal sequence comprises no exogenously introduced sequence. Such an animal may be termed a “knockout.” Also included herein are genetically modified animals in which two, three, four, five, six, seven, eight, nine, or ten or more chromosomal sequences encoding proteins associated with neurodevelopmental disorders.

In another embodiment, the genetically modified animal may comprise at least one edited chromosomal sequence encoding an orthologous protein associated with a neurodevelopmental disorder. The edited chromosomal sequence encoding an orthologous neurodevelopmental protein may be modified such that it codes for an altered protein. For example, the edited chromosomal sequence encoding a neurodevelopmental protein may comprise at least one modification such that an altered version of the protein is produced. In some embodiments, the edited chromosomal sequence comprises at least one modification such that the altered version of the neurodevelopmental protein results in a neurodevelopmental disorder in the animal. In other embodiments, the edited chromosomal sequence encoding a neurodevelopmental protein comprises at least one modification such that the altered version of the protein protects against a neurodevelopmental disorder in the animal. The modification may be a missense mutation in which substitution of one nucleotide for another nucleotide changes the identity of the coded amino acid.

In yet another embodiment, the genetically modified animal may comprise at least one chromosomally integrated sequence. The chromosomally integrated sequence may encode an orthologous neurodevelopmental protein, an endogenous neurodevelopmental protein, or combinations of both. For example, a sequence encoding an orthologous protein or an endogenous protein may be integrated into a chromosomal sequence encoding a protein such that the chromosomal sequence is inactivated, but wherein the exogenous sequence may be expressed. In such a case, the sequence encoding the orthologous protein or endogenous protein may be operably linked to a promoter control sequence. Alternatively, a sequence encoding an orthologous protein or an endogenous protein may be integrated into a chromosomal sequence without affecting expression of a chromosomal sequence. For example, a sequence encoding a neurodevelopmental protein may be integrated into a “safe harbor” locus, such as the Rosa26 locus, HPRT locus, or AAV locus. In one iteration of the disclosure an animal comprising a chromosomally integrated sequence encoding a neurodevelopmental protein may be called a “knock-in”, and it should be understood that in such an iteration of the animal, no selectable marker is present. The present disclosure also encompasses genetically modified animals in which two, three, four, five, six, seven, eight, nine, ten, eleven, twelve, thirteen, fourteen, fifteen, sixteen, seventeen, eighteen, nineteen, twenty, twenty-one, twenty-two, twenty-three or more sequences encoding protein(s) associated with neurodevelopmental disorders are integrated into the genome.

The chromosomally integrated sequence encoding a neurodevelopmental protein may encode the wild type form of the protein. Alternatively, the chromosomally integrated sequence encoding a neurodevelopmental protein may comprise at least one modification such that an altered version of the protein is produced. In some embodiments, the chromosomally integrated sequence encoding a neurodevelopmental protein comprises at least one modification such that the altered version of the protein produced causes a neurodevelopmental disorder. In other embodiments, the chromosomally integrated sequence encoding a neurodevelopmental protein comprises at least one modification such that the altered version of the protein protects against the development of a neurodevelopmental disorder.

In yet another embodiment, the genetically modified animal may comprise at least one edited chromosomal sequence encoding a neurodevelopmental protein such that the expression pattern of the protein is altered. For example, regulatory regions controlling the expression of the neurodevelopmental protein, such as a promoter or transcription binding site, may be altered such that the protein is over-produced, or the tissue-specific or temporal expression of the protein is altered, or a combination thereof. Alternatively, the expression pattern of the protein may be altered using a conditional knockout system. A non-limiting example of a conditional knockout system includes a Cre-lox recombination system. A Cre-lox recombination system comprises a Cre recombinase enzyme, a site-specific DNA recombinase that can catalyze the recombination of a nucleic acid sequence between specific sites (lox sites) in a nucleic acid molecule. Methods of using this system to produce temporal and tissue specific expression are known in the art. In general, a genetically modified animal is generated with lox sites flanking a chromosomal sequence, such as a chromosomal sequence encoding a neurodevelopmental protein. The genetically modified animal comprising the lox-flanked chromosomal sequence encoding a neurodevelopmental protein may then be crossed with another genetically modified animal expressing Cre recombinase. Progeny animals comprising the lox-flanked chromosomal sequence and the Cre recombinase are then produced, and the lox-flanked chromosomal sequence encoding the protein is recombined, leading to deletion or inversion of the chromosomal sequence encoding a neurodevelopmental protein. Expression of Cre recombinase may be temporally and conditionally regulated to effect temporally and conditionally regulated recombination of the chromosomal sequence encoding a neurodevelopmental protein.

### (a) Neurodevelopmental Proteins

Neurodevelopmental proteins are a diverse set of proteins associated with the cellular and molecular mechanisms by which complex nervous systems emerge during embryonic development and throughout life. Landmarks of neural development include the birth and differentiation of neurons from stem cell precursors, the migration of immature neurons from their birthplaces in the embryo to their final positions, outgrowth of axons from neurons and guidance of the motile growth cone through the embryo towards postsynaptic partners, the generation of synapses between the newly-formed axons and their postsynaptic partners, the neuron pruning that occurs in adolescence, and finally the lifelong changes in synapses which are thought to underlie learning and memory.

In addition to implementing the numerous processes of neurodevelopment, malformations or malfunctions of numerous proteins associated with neurodevelopmental processes may result in any one or more of numerous neurodevelopmental disorders that may adversely impact communication, speech and language as well as motor function and associated physiological functions. Non-limiting examples of genetic neurodevelopmental disorders include autism spectrum disorders such as autism, Asperger syndrome, and Pediatric Autoimmune Neuropsychiatric Disorders Associated with Streptococcal infections (PANDAS); Rett syndrome; Williams syndrome; Renpenning's syndrome; fragile X syndrome; Down syndrome; Prader-Willi syndrome; Sotos syndrome; Tuberous sclerosis complex (TSC); Timothy syndrome; Joubert syndrome; holoprosencephaly; Hirschsprung's disease; intestinal neuronal dysplasia; and Williams syndrome. Other neurodevelopmental anomalies may result from injuries prior to or during early development such as traumatic brain injuries or exposure to environmental toxins such as mercury compounds, as in the case of Minamata disease. Other metabolic diseases such as diabetes mellitus or phenylketonuria that is present during early development, either in the embryo or in the mother during gestation, may induce neurodevelopmental disorders as well.

The neurodevelopmental proteins edited using ZFN-related methods may be any protein associated with a neurodevelopmental disorder, with biochemical pathways associated with a neurodevelopmental disorder, or associated with a disorder such as phenylketonuria that is closely associated with neurodevelopmental disorders.

Non-limiting examples of neurodevelopmental genes include A2BP1 [ataxin 2-binding protein 1], AADAT [aminoadipate aminotransferase], AANAT [arylalkylamine N-acetyltransferase], ABAT [4-aminobutyrate aminotransferase], ABCA1 [ATP-binding cassette, sub-family A (ABC1), member 1], ABCA13 [ATP-binding cassette, sub-family A (ABC1), member 13], ABCA2 [ATP-binding cassette, sub-family A (ABC1), member 2], ABCB1 [ATP-binding cassette, sub-family B (MDR/TAP), member 1], ABCB11 [ATP-binding cassette, sub-family B (MDR/TAP), member 11], ABCB4 [ATP-binding cassette, sub-family B (MDR/TAP), member 4], ABCB6 [ATP-binding cassette, sub-family B (MDR/TAP), member 6], ABCB7 [ATP-binding cassette, sub-family B (MDR/TAP), member 7], ABCC1 [ATP-binding cassette, sub-family C (CFTR/MRP), member 1], ABCC2 [ATP-binding cassette, sub-family C(CFTR/MRP), member 2], ABCC3 [ATP-binding cassette, sub-family C(CFTR/MRP), member 3], ABCC4 [ATP-binding cassette, sub-family C(CFTR/MRP), member 4], ABCD1 [ATP-binding cassette, sub-family D (ALD), member 1], ABCD3 [ATP-binding cassette, sub-family D (ALD), member 3], ABCG1 [ATP-binding cassette, sub-family G (WHITE), member 1], ABCC2 [ATP-binding cassette, sub-family G (WHITE), member 2], ABCC4 [ATP-binding cassette, sub-family G (WHITE), member 4], ABHD11 [abhydrolase domain containing 11], ABI1 [abl-interactor 1], ABL1 [c-abl oncogene 1, receptor tyrosine kinase], ABL2 [v-abl Abelson murine leukemia viral oncogene homolog 2 (arg, Abelson-related gene)], ABLIM1 [actin binding LIM protein 1], ABLIM2 [actin binding LIM protein family, member 2], ABLIM3 [actin binding LIM protein family, member 3], ABO [ABO blood group (transferase A, alpha 1-3-N-acetylgalactosaminyltransferase; transferase B, alpha 1-3-galactosyltransferase)], ACAA1 [acetyl-Coenzyme A acyltransferase 1], ACACA [acetyl-Coenzyme A carboxylase alpha], ACACB [acetyl-Coenzyme A carboxylase beta], ACADL [acyl-Coenzyme A dehydrogenase, long chain], ACADM [acyl-Coenzyme A dehydrogenase, C-4 to C-12 straight chain], ACADS [acyl-Coenzyme A dehydrogenase, C-2 to C-3 short chain], ACADSB [acyl-Coenzyme A dehydrogenase, short/branched chain], ACAN [aggrecan], ACAT2 [acetyl-Coenzyme A acetyltransferase 2], ACCN1 [amiloride-sensitive cation channel 1, neuronal], ACE [angiotensin I converting enzyme (peptidyl-dipeptidase A) 1], ACE2 [angiotensin I converting enzyme (peptidyl-dipeptidase A) 2], ACHE [acetylcholinesterase (Yt blood group)], ACLY [ATP citrate lyase], ACO1 [aconitase 1, soluble], ACTA1 [actin, alpha 1, skeletal muscle], ACTB [actin, beta], ACTC1 [actin, alpha, cardiac muscle 1], ACTG1 [actin, gamma 1], ACTL6A [actin-like 6A], ACTL6B [actin-like 6B], ACTN1 [actinin, alpha 1], ACTR1A [ARP1 actin-related protein 1 homolog A, centractin alpha (yeast)], ACTR2 [ARP2 actin-related protein 2 homolog (yeast)], ACTR3 [ARP3 actin-related protein 3 homolog (yeast)], ACTR3B [ARP3 actin-related protein 3 homolog B (yeast)], ACVR1 [activin A receptor, type I], ACVR2A [activin A receptor, type IIA], ADA [adenosine deaminase], ADAM10 [ADAM metallopeptidase domain 10], ADAM11 [ADAM metallopeptidase domain 11], ADAM12 [ADAM metallopeptidase domain 12], ADAM15 [ADAM metallopeptidase domain 15], ADAM17 [ADAM metallopeptidase domain 17], ADAM18 [ADAM metallopeptidase domain 18], ADAM19 [ADAM metallopeptidase domain 19 (meltrin beta)], ADAM2 [ADAM metallopeptidase domain 2], ADAM20 [ADAM metallopeptidase domain 20], ADAM21 [ADAM metallopeptidase domain 21], ADAM22 [ADAM metallopeptidase domain 22], ADAM23 [ADAM metallopeptidase domain 23], ADAM28 [ADAM metallopeptidase domain 28], ADAM29 [ADAM metallopeptidase domain 29], ADAM30 [ADAM metallopeptidase domain 30], ADAM8 [ADAM metallopeptidase domain 8], ADAMS [ADAM metallopeptidase domain 9 (meltrin gamma)], ADAMTS1 [ADAM metallopeptidase with thrombospondin type 1 motif, 1], ADAMTS13 [ADAM metallopeptidase with thrombospondin type 1 motif, 13], ADAMTS4 [ADAM metallopeptidase with thrombospondin type 1 motif, 4], ADAMTS5 [ADAM metallopeptidase with thrombospondin type 1 motif, 5], ADAP2 [ArfGAP with dual PH domains 2], ADAR [adenosine deaminase, RNA-specific], ADARB1 [adenosine deaminase, RNA-specific, B1 (RED1 homolog rat)], ADCY1 [adenylate cyclase 1 (brain)], ADCY10 [adenylate cyclase 10 (soluble)], ADCYAP1 [adenylate cyclase activating polypeptide 1 (pituitary)], ADD1 [adducin 1 (alpha)], ADD2 [adducin 2 (beta)], ADH1A [alcohol dehydrogenase 1A (class I), alpha polypeptide], ADIPOQ [adiponectin, C1Q and collagen domain containing], ADK [adenosine kinase], ADM [adrenomedullin], ADNP [activity-dependent neuroprotector homeobox], ADORA1 [adenosine A1 receptor], ADORA2A [adenosine A2a receptor], ADORA2B [adenosine A2b receptor], ADORA3 [adenosine A3 receptor], ADRA1 B [adrenergic, alpha-1 B-, receptor], ADRA2A [adrenergic, alpha-2A-, receptor], ADRA2B [adrenergic, alpha-2B-, receptor], ADRA2C [adrenergic, alpha-2C—, receptor], ADRB1 [adrenergic, beta-1-, receptor], ADRB2 [adrenergic, beta-2-, receptor, surface], ADRB3 [adrenergic, beta-3-, receptor], ADRBK2 [adrenergic, beta, receptor kinase 2], ADSL [adenylosuccinate lyase], AFF2 [AF4/FMR2 family, member 2], AFM [afamin], AFP [alpha-fetoprotein], AGAP1 [ArfGAP with GTPase domain, ankyrin repeat and PH domain 1], AGER [advanced glycosylation end product-specific receptor], AGFG1 [ArfGAP with FG repeats 1], AGPS [alkylglycerone phosphate synthase], AGRN [agrin], AGRP [agouti related protein homolog (mouse)], AGT [angiotensinogen (serpin peptidase inhibitor, Glade A, member 8)], AGTR1 [angiotensin II receptor, type 1], AGTR2 [angiotensin II receptor, type 2], AHOY [adenosylhomocysteinase], AHI1 [Abelson helper integration site 1], AHR [aryl hydrocarbon receptor], AHSG [alpha-2-HS-glycoprotein], AICDA [activation-induced cytidine deaminase], AIFM1 [apoptosis-inducing factor, mitochondrion-associated, 1], AIRE [autoimmune regulator], AKAP12 [A kinase (PRKA) anchor protein 12], AKAP9 [A kinase (PRKA) anchor protein (yotiao) 9], AKR1A1 [aldo-keto reductase family 1, member A1 (aldehyde reductase)], AKR1B1 [aldo-keto reductase family 1, member B1 (aldose reductase)], AKR1C3 [aldo-keto reductase family 1, member C3 (3-alpha hydroxysteroid dehydrogenase, type II)], AKT1 [v-akt murine thymoma viral oncogene homolog 1], AKT2 [v-akt murine thymoma viral oncogene homolog 2], AKT3 [v-akt murine thymoma viral oncogene homolog 3 (protein kinase B, gamma)], ALAD [aminolevulinate, delta-, dehydratase], ALB [albumin], ALB [albumin], ALCAM [activated leukocyte cell adhesion molecule], ALDH1A1 [aldehyde dehydrogenase 1 family, member A1], ALDH3A1 [aldehyde dehydrogenase 3 family, member A1], ALDH5A1 [aldehyde dehydrogenase 5 family, member A1], ALDH7A1 [aldehyde dehydrogenase 7 family, member A1], ALDH9A1 [aldehyde dehydrogenase 9 family, member A1], ALDOA [aldolase A, fructose-bisphosphate], ALDOB [aldolase B, fructose-bisphosphate], ALDOC [aldolase C, fructose-bisphosphate], ALK [anaplastic lymphoma receptor tyrosine kinase], ALOX12 [arachidonate 12-lipoxygenase], ALOX5 [arachidonate 5-lipoxygenase], ALOX5AP [arachidonate 5-lipoxygenase-activating protein], ALPI [alkaline phosphatase, intestinal], ALPL [alkaline phosphatase, liver/bone/kidney], ALPP [alkaline phosphatase, placental (Regan isozyme)], ALS2 [amyotrophic lateral sclerosis 2 (juvenile)], AMACR [alpha-methylacyl-CoA racemase], AMBP [alpha-1-microglobulin/bikunin precursor], AMPH [amphiphysin], ANG [angiogenin, ribonuclease, RNase A family, 5], ANGPT1 [angiopoietin 1], ANGPT2 [angiopoietin 2], ANGPTL3 [angiopoietin-like 3], ANK1 [ankyrin 1, erythrocytic], ANK3 [ankyrin 3, node of Ranvier (ankyrin G)], ANKRD1 [ankyrin repeat domain 1 (cardiac muscle)], ANP32E [acidic (leucine-rich) nuclear phosphoprotein 32 family, member E], ANPEP [alanyl (membrane) aminopeptidase], ANXA1 [annexin A1], ANXA2 [annexin A2], ANXA5 [annexin A5], AP1S1 [adaptor-related protein complex 1, sigma 1 subunit], AP1S2 [adaptor-related protein complex 1, sigma 2 subunit], AP2A1 [adaptor-related protein complex 2, alpha 1 subunit], AP2B1 [adaptor-related protein complex 2, beta 1 subunit], APAF1 [apoptotic peptidase activating factor 1], APBA1 [amyloid beta (A4) precursor protein-binding, family A, member 1], APBA2 [amyloid beta (A4) precursor protein-binding, family A, member 2], APBB1 [amyloid beta (A4) precursor protein-binding, family B, member 1 (Fe65)], APBB2 [amyloid beta (A4) precursor protein-binding, family B, member 2], APC [adenomatous polyposis coli], APCS [amyloid P component, serum], APEX1 [APEX nuclease (multifunctional DNA repair enzyme) 1], APH1 B [anterior pharynx defective 1 homolog B (C. elegans)], APLP1 [amyloid beta (A4) precursor-like protein 1], APOA1 [apolipoprotein A-I], APOA5 [apolipoprotein A-V], APOB [apolipoprotein B (including Ag(x) antigen)], APOC2 [apolipoprotein C-II], APOD [apolipoprotein D], APOE [apolipoprotein E], APOM [apolipoprotein M], APP [amyloid beta (A4) precursor protein], APPL1 [adaptor protein, phosphotyrosine interaction, PH domain and leucine zipper containing 1], APRT [adenine phosphoribosyltransferase], APTX [aprataxin], AQP1 [aquaporin 1 (Colton blood group)], AQP2 [aquaporin 2 (collecting duct)], AQP3 [aquaporin 3 (Gill blood group)], AQP4 [aquaporin 4], AR [androgen receptor], ARC [activity-regulated cytoskeleton-associated protein], AREG [amphiregulin], ARFGEF2 [ADP-ribosylation factor guanine nucleotide-exchange factor 2 (brefeldin A-inhibited)], ARG1 [arginase, liver], ARHGAP1 [Rho GTPase activating protein 1], ARHGAP32 [Rho GTPase activating protein 32], ARHGAP4 [Rho GTPase activating protein 4], ARHGAP5 [Rho GTPase activating protein 5], ARHGDIA [Rho GDP dissociation inhibitor (GDI) alpha], ARHGEF1 [Rho guanine nucleotide exchange factor (GEF) 1], ARHGEF10 [Rho guanine nucleotide exchange factor (GEF) 10], ARHGEF11 [Rho guanine nucleotide exchange factor (GEF) 11], ARHGEF12 [Rho guanine nucleotide exchange factor (GEF) 12], ARHGEF15 [Rho guanine nucleotide exchange factor (GEF) 15], ARHGEF16 [Rho guanine nucleotide exchange factor (GEF) 16], ARHGEF2 [Rho/Rac guanine nucleotide exchange factor (GEF) 2], ARHGEF3 [Rho guanine nucleotide exchange factor (GEF) 3], ARHGEF4 [Rho guanine nucleotide exchange factor (GEF) 4], ARHGEF5 [Rho guanine nucleotide exchange factor (GEF) 5], ARHGEF6 [Rac/Cdc42 guanine nucleotide exchange factor (GEF) 6], ARHGEF7 [Rho guanine nucleotide exchange factor (GEF) 7], ARHGEF9 [Cdc42 guanine nucleotide exchange factor (GEF) 9], ARID1A [AT rich interactive domain 1A (SWI-like)], ARID1B [AT rich interactive domain 1B (SWI1-like)], ARL13B [ADP-ribosylation factor-like 13B], ARPC1A [actin related protein ⅔ complex, subunit 1A, 41 kDa], ARPC1 B [actin related protein ⅔ complex, subunit 1 B, 41 kDa], ARPC2 [actin related protein ⅔ complex, subunit 2, 34 kDa], ARPC3 [actin related protein ⅔ complex, subunit 3, 21 kDa], ARPC4 [actin related protein ⅔ complex, subunit 4, kDa], ARPC5 [actin related protein ⅔ complex, subunit 5, 16 kDa], ARPC5L [actin related protein ⅔ complex, subunit 5-like], ARPP19 [cAMP-regulated phosphoprotein, 19 kDa], ARR3 [arrestin 3, retinal (X-arrestin)], ARRB2 [arrestin, beta 2], ARSA [arylsulfatase A], ARTN [artemin], ARX [aristaless related homeobox], ASCL1 [achaetescute complex homolog 1 (Drosophila)], ASMT [acetylserotonin O-methyltransferase], ASPA [aspartoacylase (Canavan disease)], ASPG [asparaginase homolog (S. cerevisiae)], ASPH [aspartate beta-hydroxylase], ASPM [asp (abnormal spindle) homolog, microcephaly associated (Drosophila)], ASRGL1 [asparaginase like 1], ASS1 [argininosuccinate synthase 1], ASTN1 [astrotactin 1], ATAD5 [ATPase family, AAA domain containing 5], ATF2 [activating transcription factor 2], ATF4 [activating transcription factor 4 (tax-responsive enhancer element B67)], ATF6 [activating transcription factor 6], ATM [ataxia telangiectasia mutated], ATOH1 [atonal homolog 1 (Drosophila)], ATOX1 [ATX1 antioxidant protein 1 homolog (yeast)], ATP10A [ATPase, class V, type 10A], ATP2A2 [ATPase, Ca++ transporting, cardiac muscle, slow twitch 2], ATP2B2 [ATPase, Ca++ transporting, plasma membrane 2], ATP2B4 [ATPase, Ca++ transporting, plasma membrane 4], ATP50 [ATP synthase, H+ transporting, mitochondrial F1 complex, 0 subunit], ATP6AP1 [ATPase, H+ transporting, lysosomal accessory protein 1], ATP6VOC [ATPase, H+ transporting, lysosomal 16 kDa, VO subunit c], ATP7A [ATPase, Cu++ transporting, alpha polypeptide], ATP8A1 [ATPase, aminophospholipid transporter (APLT), class I, type 8A, member 1], ATR [ataxia telangiectasia and Rad3 related], ATRN [attractin], ATRX [alpha thalassemia/mental retardation syndrome X-linked (RAD54 homolog, S. cerevisiae)], ATXN1 [ataxin 1], ATXN2 [ataxin 2], ATXN3 [ataxin 3], AURKA [aurora kinase A], AUTS2 [autism susceptibility candidate 2], AVP [arginine vasopressin], AVPR1A [arginine vasopressin receptor 1A], AXIN2 [axin 2], AXL [AXL receptor tyrosine kinase], AZU1 [azurocidin 1], B2M [beta-2-microglobulin], B3GNT2 [UDP-GlcNAc:betaGal beta-1 [3-N-acetylglucosaminyltransferase 2], B9D1 [B9 protein domain 1], BACE1 [beta-site APP-cleaving enzyme 1], BACE2 [beta-site APP-cleaving enzyme 2], BACH1 [BTB and CNC homology 1, basic leucine zipper transcription factor 1], BAD [BCL2-associated agonist of cell death], BACE2 [B melanoma antigen family, member 2], BAIAP2 [BAI1-associated protein 2], BAIAP2L1 [BAI1-associated protein 2-like 1], BAK1 [BCL2-antagonist/killer 1], BARD1 [BRCA1 associated RING domain 1], BARHL1 [BarH-like homeobox 1], BARHL2 [BarH-like homeobox 2], BASP1 [brain abundant, membrane attached signal protein 1], BAX [BCL2-associated X protein], BAZ1A [bromodomain adjacent to zinc finger domain, 1A], BAZ1 B [bromodomain adjacent to zinc finger domain, 1 B], BBS9 [Bardet-Biedl syndrome 9], BCAR1 [breast cancer anti-estrogen resistance 1], BCHE [butyrylcholinesterase], BCL10 [B-cell CLL/lymphoma 10], BCL2 [B-cell CLL/lymphoma 2], BCL2A1 [BCL2-related protein A1], BCL2L1 [BCL2-like 1], BCL2L11 [BCL2-like 11 (apoptosis facilitator)], BCL3 [B-cell CLL/lymphoma 3], BCL6 [B-cell CLL/lymphoma 6], BCL7A [B-cell CLL/lymphoma 7A], BCL7B [B-cell CLL/lymphoma 7B], BCL7C [B-cell CLL/lymphoma 70], BCR [breakpoint cluster region], BDKRB1 [bradykinin receptor B1], BDNF [brain-derived neurotrophic factor], BECN1 [beclin 1, autophagy related], BEST1 [bestrophin 1], BEX1 [brain expressed, X-linked 1], BEX2 [brain expressed X-linked 2], BGLAP [bone gamma-carboxyglutamate (gla) protein], BGN [biglycan], BID [BH3 interacting domain death agonist], BIN1 [bridging integrator 1], BIRC2 [baculoviral IAP repeat-containing 2], BIRC3 [baculoviral IAP repeat-containing 3], BIRC5 [baculoviral IAP repeat-containing 5], BIRC7 [baculoviral IAP repeat-containing 7], BLK [B lymphoid tyrosine kinase], BLVRB [biliverdin reductase B (flavin reductase (NADPH))], BMI1 [BMI1 polycomb ring finger oncogene], BMP1 [bone morphogenetic protein 1], BMP10 [bone morphogenetic protein 10], BMP15 [bone morphogenetic protein 15], BMP2 [bone morphogenetic protein 2], BMP3 [bone morphogenetic protein 3], BMP4 [bone morphogenetic protein 4], BMP5 [bone morphogenetic protein 5], BMP6 [bone morphogenetic protein 6], BMP7 [bone morphogenetic protein 7], BMP8A [bone morphogenetic protein 8a], BMP8B [bone morphogenetic protein 8b], BMPR1A [bone morphogenetic protein receptor, type IA], BMPR1B [bone morphogenetic protein receptor, type IB], BMPR2 [bone morphogenetic protein receptor, type II (serine/threonine kinase)], BOO [Boc homolog (mouse)], BOK [BCL2-related ovarian killer], BPI [bactericidal/permeability-increasing protein], BRAF [v-raf murine sarcoma viral oncogene homolog B1], BRCA1 [breast cancer 1, early onset], BRCA2 [breast cancer 2, early onset], BRWD1 [bromodomain and WD repeat domain containing 1], BSND [Bartter syndrome, infantile, with sensorineural deafness (Barttin)], BST2 [bone marrow stromal cell antigen 2], BTBD10 [BTB (POZ) domain containing 10], BTC [betacellulin], BTD [biotimidase], BTG3 [BTG family, member 3], BTK [Bruton agammaglobulinemia tyrosine kinase], BTN1A1 [butyrophilin, subfamily 1, member A1], BUB1B [budding uninhibited by benzimidazoles 1 homolog beta (yeast)], 015orf2 [chromosome 15 open reading frame 2], C16orf75 [chromosome 16 open reading frame 75], C17orf42 [chromosome 17 open reading frame 42], C1orf187 [chromosome 1 open reading frame 187], C1R [complement component 1, r subcomponent], C1S [complement component 1, s subcomponent], C21orf2 [chromosome 21 open reading frame 2], C21orf33 [chromosome 21 open reading frame 33], C21orf45 [chromosome 21 open reading frame 45], C21orf62 [chromosome 21 open reading frame 62], C21orf74 [chromosome 21 open reading frame 74], C3 [complement component 3], C3orf58 [chromosome 3 open reading frame 58], C4A [complement component 4A (Rodgers blood group)], C4B [complement component 4B (Chido blood group)], C5AR1 [complement component 5a receptor 1], C6orf106 [chromosome 6 open reading frame 106], C6orf25 [chromosome 6 open reading frame 25], CA1 [carbonic anhydrase I], CA2 [carbonic anhydrase II], CA3 [carbonic anhydrase III, muscle specific], CA6 [carbonic anhydrase VI], CA9 [carbonic anhydrase IX], CABIN1 [calcineurin binding protein 1], CABLES1 [CdkS and Abl enzyme substrate 1], CACNA1B [calcium channel, voltage-dependent, N type, alpha 1B subunit], CACNA1C [calcium channel, voltage-dependent, L type, alpha 1C subunit], CACNA1 G [calcium channel, voltage-dependent, T type, alpha 1G subunit], CACNA1H [calcium channel, voltage-dependent, T type, alpha 1H subunit], CACNA2D1 [calcium channel, voltage-dependent, alpha 2/delta subunit 1], CADM1 [cell adhesion molecule 1], CADPS2 [Ca++-dependent secretion activator 2], CALB2 [calbindin 2], CALCA [calcitonin-related polypeptide alpha], CALCR [calcitonin receptor], CALM3 [calmodulin 3 (phosphorylase kinase, delta)], CALR [calreticulin], CAMK1 [calcium/calmodulin-dependent protein kinase I], CAMK2A [calcium/calmodulin-dependent protein kinase II alpha], CAMK2B [calcium/calmodulin-dependent protein kinase II beta], CAMK2G [calcium/calmodulin-dependent protein kinase II gamma], CAMK4 [calcium/calmodulin-dependent protein kinase IV], CAMKK2 [calcium/calmodulin-dependent protein kinase kinase 2, beta], CAMP [cathelicidin antimicrobial peptide], CANT1 [calcium activated nucleotidase 1], CANX [calnexin], CAPN1 [calpain 1, (mu/I) large subunit], CAPN2 [calpain 2, (m/II) large subunit], CAPN5 [calpain 5], CAPZA1 [capping protein (actin filament) muscle Z-line, alpha 1], CARD16 [caspase recruitment domain family, member 16], CARM1 [coactivator-associated arginine methyltransferase 1], CARTPT [CART prepropeptide], CASK [calcium/calmodulin-dependent serine protein kinase (MAGUK family)], CASP1 [caspase 1, apoptosis-related cysteine peptidase (interleukin 1, beta, convertase)], CASP10 [caspase 10, apoptosis-related cysteine peptidase], CASP2 [caspase 2, apoptosis-related cysteine peptidase], CASP3 [caspase 3, apoptosis-related cysteine peptidase], CASP6 [caspase 6, apoptosis-related cysteine peptidase], CASP7 [caspase 7, apoptosis-related cysteine peptidase], CASP8 [caspase 8, apoptosis-related cysteine peptidase], CASP8AP2 [caspase 8 associated protein 2], CASP9 [caspase 9, apoptosis-related cysteine peptidase], CASR [calcium-sensing receptor], CAST [calpastatin], CAT [catalase], CAV1 [caveolin 1, caveolae protein, 22 kDa], CAV2 [caveolin 2], CAV3 [caveolin 3], CBL [Cas-Br-M (murine) ecotropic retroviral transforming sequence], CBLB [Cas-Br-M (murine) ecotropic retroviral transforming sequence b], CBR1 [carbonyl reductase 1], CBR3 [carbonyl reductase 3], CBS [cystathionine-beta-synthase], CBX1 [chromobox homolog 1 (HP1 beta homolog Drosophila)], CBX5 [chromobox homolog 5 (HP1 alpha homolog, Drosophila)], CC2D2A [coiled-coil and C2 domain containing 2A], CCBE1 [collagen and calcium binding EGF domains 1], CCBL1 [cysteine conjugate-beta lyase, cytoplasmic], CCDC50 [coiled-coil domain containing 50], CCK [cholecystokinin], CCKAR [cholecystokinin A receptor], CCL1 [chemokine (C-C motif) ligand 1], CCL11 [chemokine (C-C motif) ligand 11], CCL13 [chemokine (C-C motif) ligand 13], CCL17 [chemokine (C-C motif) ligand 17], CCL19 [chemokine (C-C motif) ligand 19], CCL2 [chemokine (C-C motif) ligand 2], CCL20 [chemokine (C-C motif) ligand 20], CCL21 [chemokine (C-C motif) ligand 21], CCL22 [chemokine (C-C motif) ligand 22], CCL26 [chemokine (C-C motif) ligand 26], CCL27 [chemokine (C-C motif) ligand 27], CCL3 [chemokine (C-C motif) ligand 3], CCL4 [chemokine (C-C motif) ligand 4], CCL5 [chemokine (C-C motif) ligand 5], CCL7 [chemokine (C-C motif) ligand 7], CCL8 [chemokine (C-C motif) ligand 8], CCNA1 [cyclin A1], CCNA2 [cyclin A2], CCNB1 [cyclin B1], CCND1 [cyclin D1], CCND2 [cyclin D2], CCND3 [cyclin D3], CCNG1 [cyclin G1], CCNH [cyclin H], CCNT1 [cyclin T1], CCR1 [chemokine (C-C motif) receptor 1], CCR3 [chemokine (C-C motif) receptor 3], CCR4 [chemokine (C-C motif) receptor 4], CCR5 [chemokine (C-C motif) receptor 5], CCR6 [chemokine (C-C motif) receptor 6], CCR7 [chemokine (C-C motif) receptor 7], CCT5 [chaperonin containing TCP1, subunit 5 (epsilon)], CD14 [CD14 molecule], CD19 [CD19 molecule], CD1A [CD1a molecule], CD1B [CD1b molecule], CD1D [CD1d molecule], CD2 [CD2 molecule], CD209 [CD209 molecule], CD22 [CD22 molecule], CD244 [CD244 molecule, natural killer cell receptor 2B4], CD247 [CD247 molecule], CD27 [CD27 molecule], CD274 [CD274 molecule], CD28 [CD28 molecule], CD2AP [CD2-associated protein], CD33 [CD33 molecule], CD34 [CD34 molecule], CD36 [CD36 molecule (thrombospondin receptor)], CD3E [CD3e molecule, epsilon (CD3-TCR complex)], CD3G [CD3g molecule, gamma (CD3-TCR complex)], CD4 [CD4 molecule], CD40 [CD40 molecule, TNF receptor superfamily member 5], CD40LG [CD40 ligand], CD44 [CD44 molecule (Indian blood group)], CD46 [CD46 molecule, complement regulatory protein], CD47 [CD47 molecule], CD5 [CD5 molecule], CD55 [CD55 molecule, decay accelerating factor for complement (Cromer blood group)], CD58 [CD58 molecule], CD59 [CD59 molecule, complement regulatory protein], CD63 [CD63 molecule], CD69 [CD69 molecule], CD7 [CD7 molecule], CD72 [CD72 molecule], CD74 [CD74 molecule, major histocompatibility complex, class II invariant chain], CD79A [CD79a molecule, immunoglobulin-associated alpha], CD79B [CD79b molecule, immunoglobulin-associated beta], CD80 [CD80 molecule], CD81 [CD81 molecule], CD86 [CD86 molecule], CD8A [CD8a molecule], CD9 [CD9 molecule], CD99 [CD99 molecule], CDA [cytidine deaminase], CDC25A [cell division cycle 25 homolog A (S. pombe)], CDC25C [cell division cycle 25 homolog C (S. pombe)], CDC37 [cell division cycle 37 homolog (S. cerevisiae)], CDC42 [cell division cycle 42 (GTP binding protein, 25 kDa)], CDC5L [CDC5 cell division cycle 5-like (S. pombe)], CDH1 [cadherin 1, type 1, E-cadherin (epithelial)], CDH10 [cadherin 10, type 2 (T2-cadherin)], CDH12 [cadherin 12, type 2 (N-cadherin 2)], CDH15 [cadherin 15, type 1, M-cadherin (myotubule)], CDH2 [cadherin 2, type 1, N-cadherin (neuronal)], CDH4 [cadherin 4, type 1, R-cadherin (retinal)], CDH5 [cadherin 5, type 2 (vascular endothelium)], CDH9 [cadherin 9, type 2 (T1-cadherin)], CDIPT [CDP-diacylglycerol-inositol 3-phosphatidyltransferase (phosphatidylinositol synthase)], CDK1 [cyclin-dependent kinase 1], CDK14 [cyclin-dependent kinase 14], CDK2 [cyclin-dependent kinase 2], CDK4 [cyclin-dependent kinase 4], CDK5 [cyclin-dependent kinase 5], CDK5R1 [cyclin-dependent kinase 5, regulatory subunit 1 (p35)], CDK5RAP2 [CDK5 regulatory subunit associated protein 2], CDK6 [cyclin-dependent kinase 6], CDK7 [cyclin-dependent kinase 7], CDK9 [cyclin-dependent kinase 9], CDKL5 [cyclin-dependent kinase-like 5], CDKN1A [cyclin-dependent kinase inhibitor 1A (p21, Cip1)], CDKN1B [cyclin-dependent kinase inhibitor 1B (p27, Kip1)], CDKN1C [cyclin-dependent kinase inhibitor 1C (p57, Kip2)], CDKN2A [cyclin-dependent kinase inhibitor 2A (melanoma, p16, inhibits CDK4)], CDKN2B [cyclin-dependent kinase inhibitor 2B (p15, inhibits CDK4)], CDKN2C [cyclin-dependent kinase inhibitor 2C (p18, inhibits CDK4)], CDKN2D [cyclin-dependent kinase inhibitor 2D (p19, inhibits CDK4)], CDNF [cerebral dopamine neurotrophic factor], CDO1 [cysteine dioxygenase, type I], CDR2 [cerebellar degeneration-related protein 2, 62 kDa], CDT1 [chromatin licensing and DNA replication factor 1], CDX1 [caudal type homeobox 1], CDX2 [caudal type homeobox 2], CEACAM1 [carcinoembryonic antigen-related cell adhesion molecule 1 (biliary glycoprotein)], CEACAM3 [carcinoembryonic antigen-related cell adhesion molecule 3], CEACAM5 [carcinoembryonic antigen-related cell adhesion molecule 5], CEACAM7 [carcinoembryonic antigen-related cell adhesion molecule 7], CEBPB [CCAAT/enhancer binding protein (C/EBP), beta], CEBPD [CCAAT/enhancer binding protein (C/EBP), delta], CECR2 [cat eye syndrome chromosome region, candidate 2], CEL [carboxyl ester lipase (bile salt-stimulated lipase)], CENPC1 [centromere protein C1], CENPJ [centromere protein J], CEP290 [centrosomal protein 290 kDa], CER1 [cerberus 1, cysteine knot superfamily, homolog (Xenopus laevis)], CETP [cholesteryl ester transfer protein, plasma], CFC1 [cripto, FRL-1, cryptic family 1], CFH [complement factor H], CFHR1 [complement factor H-related 1], CFHR3 [complement factor H-related 3], CFHR4 [complement factor H-related 4], CFI [complement factor I], CFL1 [cofilin 1 (non-muscle)], CFL2 [cofilin 2 (muscle)], CFLAR [CASP8 and FADD-like apoptosis regulator], CFTR [cystic fibrosis transmembrane conductance regulator (ATP-binding cassette sub-family C, member 7)], CGA [glycoprotein hormones, alpha polypeptide], CGB [chorionic gonadotropin, beta polypeptide], CGB5 [chorionic gonadotropin, beta polypeptide 5], CGGBP1 [CGG triplet repeat binding protein 1], CHAF1A [chromatin assembly factor 1, subunit A (p150)], CHAF1 B [chromatin assembly factor 1, subunit B (p60)], CHAT [choline acetyltransferase], CHEK1 [CHK1 checkpoint homolog (S. pombe)], CHEK2 [CHK2 checkpoint homolog (S. pombe)], CHGA [chromogranin A (parathyroid secretory protein 1)], CHKA [choline kinase alpha], CHL1 [cell adhesion molecule with homology to L1CAM (close homolog of L1)], CHN1 [chimerin (chimaerin) 1], CHP [calcium binding protein P22], CHP2 [calcineurin B homologous protein 2], CHRD [chordin], CHRM1 [cholinergic receptor, muscarinic 1], CHRM2 [cholinergic receptor, muscarinic 2], CHRM3 [cholinergic receptor, muscarinic 3], CHRM5 [cholinergic receptor, muscarinic 5], CHRNA3 [cholinergic receptor, nicotinic, alpha 3], CHRNA4 [cholinergic receptor, nicotinic, alpha 4], CHRNA7 [cholinergic receptor, nicotinic, alpha 7], CHRNB2 [cholinergic receptor, nicotinic, beta 2 (neuronal)], CHST1 [carbohydrate (keratan sulfate Gal-6) sulfotransferase 1], CHST10 [carbohydrate sulfotransferase 10], CHST3 [carbohydrate (chondroitin 6) sulfotransferase 3], CHUK [conserved helix-loop-helix ubiquitous kinase], CHURC1 [churchill domain containing 1], CIB1 [calcium and integrin binding 1 (calmyrin)], CIITA [class II, major histocompatibility complex, transactivator], CIRBP [cold inducible RNA binding protein], CISD1 [CDGSH iron sulfur domain 1], CISH [cytokine inducible SH2-containing protein], CIT [citron (rho-interacting, serine/threonine kinase 21)], CLASP2 [cytoplasmic linker associated protein 2], CLCF1 [cardiotrophin-like cytokine factor 1], CLCN2 [chloride channel 2], CLDN1 [claudin 1], CLDN14 [claudin 14], CLDN16 [claudin 16], CLDN3 [claudin 3], CLDN4 [claudin 4], CLDN5 [claudin 5], CLDN8 [claudin 8], CLEC12A [C-type lectin domain family 12, member A], CLEC16A [C-type lectin domain family 16, member A], CLEC5A [C-type lectin domain family 5, member A], CLEC7A [C-type lectin domain family 7, member A], CLIP2 [CAP-GLY domain containing linker protein 2], CLSTN1 [calsyntenin 1], CLTC [clathrin, heavy chain (Hc)], CLU [clusterin], CMIP [c-Maf-inducing protein], CNBP [CCHC-type zinc finger, nucleic acid binding protein], CNGA3 [cyclic nucleotide gated channel alpha 3], CNGB3 [cyclic nucleotide gated channel beta 3], CNN1 [calponin 1, basic, smooth muscle], CNN2 [calponin 2], CNN3 [calponin 3, acidic], CNOT8 [CCR4—NOT transcription complex, subunit 8], CNP [2′[3′-cyclic nucleotide 3′ phosphodiesterase], CNR1 [cannabinoid receptor 1 (brain)], CNR2 [cannabinoid receptor 2 (macrophage)], CNTF [ciliary neurotrophic factor], CNTFR [ciliary neurotrophic factor receptor], CNTFR [ciliary neurotrophic factor receptor], CNTFR [ciliary neurotrophic factor receptor], CNTLN [centlein, centrosomal protein], CNTN1 [contactin 1], CNTN2 [contactin 2 (axonal)], CNTN4 [contactin 4], CNTNAP1 [contactin associated protein 1], CNTNAP2 [contactin associated protein-like 2], COBL [cordon-bleu homolog (mouse)], COG2 [component of oligomeric golgi complex 2], COL18A1 [collagen, type XVIII, alpha 1], COL1A1 [collagen, type I, alpha 1], COL1A2 [collagen, type I, alpha 2], COL2A1 [collagen, type II, alpha 1], COL3A1 [collagen, type III, alpha 1], COL4A3 [collagen, type IV, alpha 3 (Goodpasture antigen)], COL4A3BP [collagen, type IV, alpha 3 (Goodpasture antigen) binding protein], COL5A1 [collagen, type V, alpha 1], COL5A2 [collagen, type V, alpha 2], COL6A1 [collagen, type VI, alpha 1], COL6A2 [collagen, type VI, alpha 2], COL6A3 [collagen, type VI, alpha 3], COMT [catechol-O-methyltransferase], COPG2 [coatomer protein complex, subunit gamma 2], COPS4 [COPS constitutive photomorphogenic homolog subunit 4 (Arabidopsis)], CORO1A [coronin, actin binding protein, 1A], COX5A [cytochrome c oxidase subunit Va], COX7B [cytochrome c oxidase subunit VIIb], CP [ceruloplasmin (ferroxidase)], CPA1 [carboxypeptidase A1 (pancreatic)], CPA2 [carboxypeptidase A2 (pancreatic)], CPA5 [carboxypeptidase A5], CPB2 [carboxypeptidase B2 (plasma)], CPDX [coproporphyrinogen oxidase], CPS1 [carbamoyl-phosphate synthetase 1, mitochondrial], CPT1A [carnitine palmitoyltransferase 1A (liver)], CR1 [complement component (3b/4b) receptor 1 (Knops blood group)], CR2 [complement component (3d/Epstein Barr virus) receptor 2], CRABP1 [cellular retinoic acid binding protein 1], CRABP2 [cellular retinoic acid binding protein 2], CRAT [carnitine O-acetyltransferase], CRB1 [crumbs homolog 1 (Drosophila)], CREB1 [cAMP responsive element binding protein 1], CREBBP [CREB binding protein], CRELD1 [cysteine-rich with EGF-like domains 1], CRH [corticotropin releasing hormone], CRIP1 [cysteine-rich protein 1 (intestinal)], CRK [v-crk sarcoma virus CT10 oncogene homolog (avian)], CRKL [v-crk sarcoma virus CT10 oncogene homolog (avian)-like], CRLF1 [cytokine receptor-like factor 1], CRLF2 [cytokine receptor-like factor 2], CRLF3 [cytokine receptor-like factor 3], CRMP1 [collapsin response mediator protein 1], CRP [C-reactive protein, pentraxin-related], CRTC1 [CREB regulated transcription coactivator 1], CRX [cone-rod homeobox], CRYAA [crystallin, alpha A], CRYAB [crystallin, alpha B], CS [citrate synthase], CSAD [cysteine sulfinic acid decarboxylase], CSF1 [colony stimulating factor 1 (macrophage)], CSF1 R [colony stimulating factor 1 receptor], CSF2 [colony stimulating factor 2 (granulocyte-macrophage)], CSF2RA [colony stimulating factor 2 receptor, alpha, low-affinity (granulocyte-macrophage)], CSF3 [colony stimulating factor 3 (granulocyte)], CSF3R [colony stimulating factor 3 receptor (granulocyte)], CSH2 [chorionic somatomammotropin hormone 2], CSK [c-src tyrosine kinase], CSMD1 [CUB and Sushi multiple domains 1], CSMD3 [CUB and Sushi multiple domains 3], CSNK1D [casein kinase 1, delta], CSNK1E [casein kinase 1, epsilon], CSNK2A1 [casein kinase 2, alpha 1 polypeptide], CSPG4 [chondroitin sulfate proteoglycan 4], CSPG5 [chondroitin sulfate proteoglycan 5 (neuroglycan C)], CST3 [cystatin C], CST7 [cystatin F (leukocystatin)], CSTB [cystatin B (stefin B)], CTAG1 B [cancer/testis antigen 1 B], CTBP1 [C-terminal binding protein 1], CTCF [CCCTC-binding factor (zinc finger protein)], CTDSP1 [CTD (carboxy-terminal domain, RNA polymerase II, polypeptide A) small phosphatase 1], CTF1 [cardiotrophin 1], CTGF [connective tissue growth factor], CTLA4 [cytotoxic T-lymphocyte-associated protein 4], CTNNA1 [catenin (cadherin-associated protein), alpha 1, 102 kDa], CTNNAL1 [catenin (cadherin-associated protein), alpha-like 1], CTNNB1 [catenin (cadherin-associated protein), beta 1, 88 kDa], CTNND1 [catenin (cadherin-associated protein), delta 1], CTNND2 [catenin (cadherin-associated protein), delta 2 (neural plakophilin-related arm-repeat protein)], CTNS [cystinosis, nephropathic], CTRL [chymotrypsin-like], CTSB [cathepsin B], CTSC [cathepsin C], CTSD [cathepsin D], CTSG [cathepsin G], CTSH [cathepsin H], CTSL1 [cathepsin L1], CTSS [cathepsin S], CTTN [cortactin], CTTNBP2 [cortactin binding protein 2], CUL4B [cullin 4B], CUL5 [cullin 5], CUX2 [cut-like homeobox 2], CX3CL1 [chemokine (C-X3-C motif) ligand 1], CX3CR1 [chemokine (C-X3-C motif) receptor 1], CXADR [coxsackie virus and adenovirus receptor], CXCL1 [chemokine (C-X-C motif) ligand 1 (melanoma growth stimulating activity, alpha)], CXCL10 [chemokine (C-X-C motif) ligand 10], CXCL12 [chemokine (C-X-C motif) ligand 12 (stromal cell-derived factor 1)], CXCL16 [chemokine (C-X-C motif) ligand 16], CXCL2 [chemokine (C-X-C motif) ligand 2], CXCL5 [chemokine (C-X-C motif) ligand 5], CXCR1 [chemokine (C-X-C motif) receptor 1], CXCR2 [chemokine (C-X-C motif) receptor 2], CXCR3 [chemokine (C-X-C motif) receptor 3], CXCR4 [chemokine (C-X-C motif) receptor 4], CXCR5 [chemokine (C-X-C motif) receptor 5], CYB5A [cytochrome b5 type A (microsomal)], CYBA [cytochrome b-245, alpha polypeptide], CYBB [cytochrome b-245, beta polypeptide], CYCS [cytochrome c, somatic], CYFIP1 [cytoplasmic FMR1 interacting protein 1], CYLD [cylindromatosis (turban tumor syndrome)], CYP11A1 [cytochrome P450, family 11, subfamily A, polypeptide 1], CYP11B1 [cytochrome P450, family 11, subfamily B, polypeptide 1], CYP11B2 [cytochrome P450, family 11, subfamily B, polypeptide 2], CYP17A1 [cytochrome P450, family 17, subfamily A, polypeptide 1], CYP19A1 [cytochrome P450, family 19, subfamily A, polypeptide 1], CYP1A1 [cytochrome P450, family 1, subfamily A, polypeptide 1], CYP1A2 [cytochrome P450, family 1, subfamily A, polypeptide 2], CYP1B1 [cytochrome P450, family 1, subfamily B, polypeptide 1], CYP21A2 [cytochrome P450, family 21, subfamily A, polypeptide 2], CYP2A6 [cytochrome P450, family 2, subfamily A, polypeptide 6], CYP2B6 [cytochrome P450, family 2, subfamily B, polypeptide 6], CYP2C9 [cytochrome P450, family 2, subfamily C, polypeptide 9], CYP2D6 [cytochrome P450, family 2, subfamily D, polypeptide 6], CYP2E1 [cytochrome P450, family 2, subfamily E, polypeptide 1], CYP3A4 [cytochrome P450, family 3, subfamily A, polypeptide 4], CYP7A1 [cytochrome P450, family 7, subfamily A, polypeptide 1], CYR61 [cysteine-rich, angiogenic inducer, 61], CYSLTR1 [cysteinyl leukotriene receptor 1], CYSLTR2 [cysteinyl leukotriene receptor 2], DAB1 [disabled homolog 1 (Drosophila)], DAGLA [diacylglycerol lipase, alpha], DAGLB [diacylglycerol lipase, beta], DAO [D-amino-acid oxidase], DAOA [D-amino acid oxidase activator], DAPK1 [death-associated protein kinase 1], DAPK3 [death-associated protein kinase 3], DAXX [death-domain associated protein], DBH [dopamine beta-hydroxylase (dopamine beta-monooxygenase)], DBI [diazepam binding inhibitor (GABA receptor modulator, acyl-Coenzyme A binding protein)], DBN1 [drebrin 1], DCAF6 [DDB1 and CUL4 associated factor 6], DCC [deleted in colorectal carcinoma], DCDC2 [doublecortin domain containing 2], DCK [deoxycytidine kinase], DCLK1 [doublecortin-like kinase 1], DCN [decorin], DCTN1 [dynactin 1 (p150, glued homolog, Drosophila)], DCTN2 [dynactin 2 (p50)], DCTN4 [dynactin 4 (p62)], DCUN1D1 [DCN1, defective in cullin neddylation 1, domain containing 1 (S. cerevisiae)], DCX [doublecortin], DDB1 [damage-specific DNA binding protein 1, 127 kDa], DDC [dopa decarboxylase (aromatic L-amino acid decarboxylase)], DDIT3 [DNA-damage-inducible transcript 3], DDIT4 [DNA-damage-inducible transcript 4], DDIT4L [DNA-damage-inducible transcript 4-like], DDR1 [discoidin domain receptor tyrosine kinase 1], DDX10 [DEAD (Asp-Glu-Ala-Asp) box polypeptide 10], DDX17 [DEAD (Asp-Glu-Ala-Asp) box polypeptide 17], DEFB4A [defensin, beta 4A], DEK [DEK oncogene], DES [desmin], DEXI [Dexi homolog (mouse)], DFFA [DNA fragmentation factor, 45 kDa, alpha polypeptide], DFNB31 [deafness, autosomal recessive 31], DGCR6 [DiGeorge syndrome critical region gene 6], DGUOK [deoxyguanosine kinase], DHCR7 [7-dehydrocholesterol reductase], DHFR [dihydrofolate reductase], DIAPH1 [diaphanous homolog 1 (Drosophila)], DICER1 [dicer 1, ribonuclease type III], D101 [deiodinase, iodothyronine, type I], D102 [deiodinase, iodothyronine, type II], DIP2A [DIP2 disco-interacting protein 2 homolog A (Drosophila)], DIRAS3 [DIRAS family, GTP-binding RAS-like 3], DISCI [disrupted in schizophrenia 1], DISC2 [disrupted in schizophrenia 2 (non-protein coding)], DKC1 [dyskeratosis congenita 1, dyskerin], DLG1 [discs, large homolog 1 (Drosophila)], DLG2 [discs, large homolog 2 (Drosophila)], DLG3 [discs, large homolog 3 (Drosophila)], DLG4 [discs, large homolog 4 (Drosophila)], DLGAP1 [discs, large (Drosophila) homolog-associated protein 1], DLGAP2 [discs, large (Drosophila) homolog-associated protein 2], DLK1 [delta-like 1 homolog (Drosophila)], DLL1 [delta-like 1 (Drosophila)], DLX1 [distal-less homeobox 1], DLX2 [distal-less homeobox 2], DLX3 [distal-less homeobox 3], DLX4 [distal-less homeobox 4], DLX5 [distal-less homeobox 5], DLX6 [distal-less homeobox 6], DMBT1 [deleted in malignant brain tumors 1], DMC1 [DMC1 dosage suppressor of mck1 homolog, meiosis-specific homologous recombination (yeast)], DMD [dystrophin], DMPK [dystrophia myotonica-protein kinase], DNAI2 [dynein, axonemal, intermediate chain 2], DNAJC28 [DnaJ (Hsp40) homolog, subfamily C, member 28], DNAJC30 [DnaJ (Hsp40) homolog, subfamily C, member 30], DNASE1 [deoxyribonuclease I], DNER [delta/notch-like EGF repeat containing], DNLZ [DNL-type zinc finger], DNM1 [dynamin 1], DNM3 [dynamin 3], DNMT1 [DNA (cytosine-5-)-methyltransferase 1], DNMT3A [DNA (cytosine-5-)-methyltransferase 3 alpha], DNMT3B [DNA (cytosine-5-)-methyltransferase 3 beta], DNTT [deoxynucleotidyltransferase, terminal], DOC2A [double C2-like domains, alpha], DOCK1 [dedicator of cytokinesis 1], DOCK3 [dedicator of cytokinesis 3], DOCK4 [dedicator of cytokinesis 4], DOCK7 [dedicator of cytokinesis 7], DOK7 [docking protein 7], DONSON [downstream neighbor of SON], DOPEY1 [dopey family member 1], DOPEY2 [dopey family member 2], DPF1 [D4, zinc and double PHD fingers family 1], DPF3 [D4, zinc and double PHD fingers, family 3], DPH1 [DPH1 homolog (S. cerevisiae)], DPP10 [dipeptidyl-peptidase 10], DPP4 [dipeptidyl-peptidase 4], DPRXP4 [divergent-paired related homeobox pseudogene 4], DPT [dermatopontin], DPYD [dihydropyrimidine dehydrogenase], DPYSL2 [dihydropyrimidinase-like 2], DPYSL3 [dihydropyrimidinase-like 3], DPYSL4 [dihydropyrimidinase-like 4], DPYSL5 [dihydropyrimidinase-like 5], DRD1 [dopamine receptor D1], DRD2 [dopamine receptor D2], DRD3 [dopamine receptor D3], DRD4 [dopamine receptor D4], DRD5 [dopamine receptor D5], DRG1 [developmentally regulated GTP binding protein 1], DRGX [dorsal root ganglia homeobox], DSC2 [desmocollin 2], DSCAM [Down syndrome cell adhesion molecule], DSCAML1 [Down syndrome cell adhesion molecule like 1], DSCR3 [Down syndrome critical region gene 3], DSCR4 [Down syndrome critical region gene 4], DSCR6 [Down syndrome critical region gene 6], DSERG1 [Down syndrome encephalopathy related protein 1], DSG1 [desmoglein 1], DSG2 [desmoglein 2], DSP [desmoplakin], DST [dystonin], DSTN [destrin (actin depolymerizing factor)], DTNBP1 [dystrobrevin binding protein 1], DULLARD [dullard homolog (Xenopus laevis)], DUSP1 [dual specificity phosphatase 1], DUSP13 [dual specificity phosphatase 13], DUSP6 [dual specificity phosphatase 6], DUT [deoxyuridine triphosphatase], DVL1 [dishevelled, dsh homolog 1 (Drosophila)], DYRK1A [dual-specificity tyrosine-(Y)-phosphorylation regulated kinase 1A], DYRK3 [dual-specificity tyrosine-(Y)-phosphorylation regulated kinase 3], DYSF [dysferlin, limb girdle muscular dystrophy 2B (autosomal recessive)], DYX1C1 [dyslexia susceptibility 1 candidate 1], E2F1 [E2F transcription factor 1], EARS2 [glutamyl-tRNA synthetase 2, mitochondrial (putative)], EBF4 [early B-cell factor 4], ECE1 [endothelin converting enzyme 1], ECHS1 [enoyl Coenzyme A hydratase, short chain, 1, mitochondrial], EDN1 [endothelin 1], EDN2 [endothelin 2], EDN3 [endothelin 3], EDNRA [endothelin receptor type A], EDNRB [endothelin receptor type B], EEF1A1 [eukaryotic translation elongation factor 1 alpha 1], EEF2 [eukaryotic translation elongation factor 2], EEF2K [eukaryotic elongation factor-2 kinase], EFHA1 [EF-hand domain family, member A1], EFNA1 [ephrin-A1], EFNA2 [ephrin-A2], EFNA3 [ephrin-A3], EFNA4 [ephrin-A4], EFNA5 [ephrin-A5], EFNB2 [ephrin-B2], EFNB3 [ephrin-B3], EFS [embryonal Fyn-associated substrate], EGF [epidermal growth factor (beta-urogastrone)], EGFR [epidermal growth factor receptor (erythroblastic leukemia viral (v-erb-b) oncogene homolog, avian)], EGLN1 [egl nine homolog 1 (C. elegans)], EGR1 [early growth response 1], EGR2 [early growth response 2], EGR3 [early growth response 3], EHHADH [enoyl-Coenzyme A, hydratase/3-hydroxyacyl Coenzyme A dehydrogenase], EHMT2 [euchromatic histone-lysine N-methyltransferase 2], EID1 [EP300 interacting inhibitor of differentiation 1], EIF1AY [eukaryotic translation initiation factor 1A, Y-linked], EIF2AK2 [eukaryotic translation initiation factor 2-alpha kinase 2], EIF2AK3 [eukaryotic translation initiation factor 2-alpha kinase 3], EIF2B2 [eukaryotic translation initiation factor 2B, subunit 2 beta, 39 kDa], EIF2B5 [eukaryotic translation initiation factor 2B, subunit 5 epsilon, 82 kDa], EIF2S1 [eukaryotic translation initiation factor 2, subunit 1 alpha, 35 kDa], EIF2S2 [eukaryotic translation initiation factor 2, subunit 2 beta, 38 kDa], EIF3M [eukaryotic translation initiation factor 3, subunit M], EIF4E [eukaryotic translation initiation factor 4E], EIF4EBP1 [eukaryotic translation initiation factor 4E binding protein 1], EIF4G1 [eukaryotic translation initiation factor 4 gamma, 1], EIF4H [eukaryotic translation initiation factor 4H], ELANE [elastase, neutrophil expressed], ELAVL1 [ELAV (embryonic lethal, abnormal vision, Drosophila)-like 1 (Hu antigen R)], ELAVL3 [ELAV (embryonic lethal, abnormal vision, Drosophila)-like 3 (Hu antigen C)], ELAVL4 [ELAV (embryonic lethal, abnormal vision, Drosophila)-like 4 (Hu antigen D)], ELF5 [E74-like factor 5 (ets domain transcription factor)], ELK1 [ELK1, member of ETS oncogene family], ELMO1 [engulfment and cell motility 1], ELN [elastin], ELP4 [elongation protein 4 homolog (S. cerevisiae)], EMP2 [epithelial membrane protein 2], EMP3 [epithelial membrane protein 3], EMX1 [empty spiracles homeobox 1], EMX2 [empty spiracles homeobox 2], EN1 [engrailed homeobox 1], EN2 [engrailed homeobox 2], ENAH [enabled homolog (Drosophila)], ENDOG [endonuclease G], ENG [endoglin], ENO1 [enolase 1, (alpha)], ENO2 [enolase 2 (gamma, neuronal)], ENPEP [glutamyl aminopeptidase (aminopeptidase A)], ENPP1 [ectonucleotide pyrophosphatase/phosphodiesterase 1], ENPP2 [ectonucleotide pyrophosphatase/phosphodiesterase 2], ENSA [endosulfine alpha], ENSG00000174496 [ ], ENSG00000183653 [ ], ENSG00000215557 [ ], ENTPD1 [ectonucleoside triphosphate diphosphohydrolase 1], EP300 [E1A binding protein p300], EPCAM [epithelial cell adhesion molecule], EPHA1 [EPH receptor A1], EPHA10 [EPH receptor A10], EPHA2 [EPH receptor A2], EPHA3 [EPH receptor A3], EPHA4 [EPH receptor A4], EPHA5 [EPH receptor A5], EPHA6 [EPH receptor A6], EPHA7 [EPH receptor A7], EPHA8 [EPH receptor A8], EPHB1 [EPH receptor B1], EPHB2 [EPH receptor B2], EPHB3 [EPH receptor B3], EPHB4 [EPH receptor B4], EPHB6 [EPH receptor B6], EPHX2 [epoxide hydrolase 2, cytoplasmic], EPM2A [epilepsy, progressive myoclonus type 2A, Lafora disease (laforin)], EPO [erythropoietin], EPOR [erythropoietin receptor], EPRS [glutamyl-prolyl-tRNA synthetase], EPS15 [epidermal growth factor receptor pathway substrate 15], ERBB2 [v-erb-b2 erythroblastic leukemia viral oncogene homolog 2, neuro/glioblastoma derived oncogene homolog (avian)], ERBB3 [v-erb-b2 erythroblastic leukemia viral oncogene homolog 3 (avian)], ERBB4 [v-erb-a erythroblastic leukemia viral oncogene homolog 4 (avian)], ERC2 [ELKS/RAB6-interacting/CAST family member 2], ERCC2 [excision repair cross-complementing rodent repair deficiency, complementation group 2], ERCC3 [excision repair cross-complementing rodent repair deficiency, complementation group 3 (xeroderma pigmentosum group B complementing)], ERCC5 [excision repair cross-complementing rodent repair deficiency, complementation group 5], ERCC6 [excision repair cross-complementing rodent repair deficiency, complementation group 6], ERCC8 [excision repair cross-complementing rodent repair deficiency, complementation group 8], EREG [epiregulin], ERG [v-ets erythroblastosis virus E26 oncogene homolog (avian)], ERVWE1 [endogenous retroviral family W, env(C7), member 1], ESD [esterase D/formylglutathione hydrolase], ESR1 [estrogen receptor 1], ESR2 [estrogen receptor 2 (ER beta)], ESRRA [estrogen-related receptor alpha], ESRRB [estrogen-related receptor beta], ETS1 [v-ets erythroblastosis virus E26 oncogene homolog 1 (avian)], ETS2 [v-ets erythroblastosis virus E26 oncogene homolog 2 (avian)], ETV1 [ets variant 1], ETV4 [ets variant 4], ETV5 [ets variant 5], ETV6 [ets variant 6], EVL [Enah/Vasp-like], EXOC4 [exocyst complex component 4], EXOC8 [exocyst complex component 8], EXT1 [exostoses (multiple) 1], EXT2 [exostoses (multiple) 2], EZH2 [enhancer of zeste homolog 2 (Drosophila)], EZR [ezrin], F12 [coagulation factor XII (Hageman factor)], F2 [coagulation factor II (thrombin)], F2R [coagulation factor II (thrombin) receptor], F2RL1 [coagulation factor II (thrombin) receptor-like 1], F3 [coagulation factor III (thromboplastin, tissue factor)], F7 [coagulation factor VII (serum prothrombin conversion accelerator)], F8 [coagulation factor VIII, procoagulant component], F9 [coagulation factor IX], FAAH [fatty acid amide hydrolase], FABP3 [fatty acid binding protein 3, muscle and heart (mammary-derived growth inhibitor)], FABP4 [fatty acid binding protein 4, adipocyte], FABP5 [fatty acid binding protein 5 (psoriasis-associated)], FABP7 [fatty acid binding protein 7, brain], FADD [Fas (TNFRSF6)-associated via death domain], FADS2 [fatty acid desaturase 2], FAM120C [family with sequence similarity 120C], FAM165B [family with sequence similarity 165, member B], FAM3C [family with sequence similarity 3, member C], FAM53A [family with sequence similarity 53, member A], FARP2 [FERM, RhoGEF and pleckstrin domain protein 2], FARSA [phenylalanyl-tRNA synthetase, alpha subunit], FAS [Fas (TNF receptor superfamily, member 6)], FASLG [Fas ligand (TNF superfamily, member 6)], FASN [fatty acid synthase], FASTK [Fas-activated serine/threonine kinase], FBLN1 [fibulin 1], FBN1 [fibrillin 1], FBP1 [fructose-1 [6-bisphosphatase 1], FBXO45 [F-box protein 45], FBXW5 [F-box and WD repeat domain containing 5], FBXW7 [F-box and WD repeat domain containing 7], FCER2 [Fc fragment of IgE, low affinity II, receptor for (CD23)], FCGR1A [Fc fragment of IgG, high affinity Ia, receptor (CD64)], FCGR2A [Fc fragment of IgG, low affinity IIa, receptor (CD32)], FCGR2B [Fc fragment of IgG, low affinity IIb, receptor (CD32)], FCGR3A [Fc fragment of IgG, low affinity IIIa, receptor (CD16a)], FCRL3 [Fc receptor-like 3], FDFT1 [farnesyl-diphosphate farnesyltransferase 1], FDX1 [ferredoxin 1], FDXR [ferredoxin reductase], FECH [ferrochelatase (protoporphyria)], FEM1A [fem-1 homolog a (C. elegans)], FER [fer (fps/fes related) tyrosine kinase], FES [feline sarcoma oncogene], FEZ1 [fasciculation and elongation protein zeta 1 (zygin I)], FEZ2 [fasciculation and elongation protein zeta 2 (zygin II)], FEZF1 [FEZ family zinc finger 1], FEZF2 [FEZ family zinc finger 2], FGF1 [fibroblast growth factor 1 (acidic)], FGF19 [fibroblast growth factor 19], FGF2 [fibroblast growth factor 2 (basic)], FGF20 [fibroblast growth factor 20], FGF3 [fibroblast growth factor 3 (murine mammary tumor virus integration site (v-int-2) oncogene homolog)], FGF4 [fibroblast growth factor 4], FGF5 [fibroblast growth factor 5], FGF7 [fibroblast growth factor 7 (keratinocyte growth factor)], FGF8 [fibroblast growth factor 8 (androgen-induced)], FGF9 [fibroblast growth factor 9 (glia-activating factor)], FGFBP1 [fibroblast growth factor binding protein 1], FGFR1 [fibroblast growth factor receptor 1], FGFR2 [fibroblast growth factor receptor 2], FGFR3 [fibroblast growth factor receptor 3], FGFR4 [fibroblast growth factor receptor 4], FHIT [fragile histidine triad gene], FHL1 [four and a half L1M domains 1], FHL2 [four and a half LIM domains 2], FIBP [fibroblast growth factor (acidic) intracellular binding protein], FIGF [c-fos induced growth factor (vascular endothelial growth factor D)], FIGNL1 [fidgetin-like 1], FKBP15 [FK506 binding protein 15, 133 kDa], FKBP1B [FK506 binding protein 1B, 12.6 kDa], FKBP5 [FK506 binding protein 5], FKBP6 [FK506 binding protein 6, 36 kDa], FKBP8 [FK506 binding protein 8, 38 kDa], FKTN [fukutin], FLCN [folliculin], FLG [filaggrin], FLI1 [Friend leukemia virus integration 1], FLNA [filamin A, alpha], FLNB [filamin B, beta], FLNC [filamin C, gamma], FLT1 [fms-related tyrosine kinase 1 (vascular endothelial growth factor/vascular permeability factor receptor)], FLT3 [fms-related tyrosine kinase 3], FMN1 [formin 1], FMNL2 [formin-like 2], FMR1 [fragile X mental retardation 1], FN1 [fibronectin 1], FOLH1 [folate hydrolase (prostate-specific membrane antigen) 1], FOLR1 [folate receptor 1 (adult)], FOS [FBJ murine osteosarcoma viral oncogene homolog], FOSB [FBJ murine osteosarcoma viral oncogene homolog B], FOXC2 [forkhead box C2 (MFH-1, mesenchyme forkhead 1)], FOXG1 [forkhead box G1], FOXL2 [forkhead box L2], FOXM1 [forkhead box M1], FOXO1 [forkhead box 01], FOXO3 [forkhead box 03], FOXP2 [forkhead box P2], FOXP3 [forkhead box P3], FPR1 [formyl peptide receptor 1], FPR2 [formyl peptide receptor 2], FRMD7 [FERM domain containing 7], FRS2 [fibroblast growth factor receptor substrate 2], FRS3 [fibroblast growth factor receptor substrate 3], FRYL [FRY-like], FSCN1 [fascin homolog 1, actin-bundling protein (Strongylocentrotus purpuratus)], FSHB [follicle stimulating hormone, beta polypeptide], FSHR [follicle stimulating hormone receptor], FST [follistatin], FSTL1 [follistatin-like 1], FSTL3 [follistatin-like 3 (secreted glycoprotein)], FTCD [formiminotransferase cyclodeaminase], FTH1 [ferritin, heavy polypeptide 1], FTL [ferritin, light polypeptide], FTMT [ferritin mitochondrial], FTSJ1 [FtsJ homolog 1 (E. coli)], FUCA1 [fucosidase, alpha-L-1, tissue], FURIN [furin (paired basic amino acid cleaving enzyme)], FUT1 [fucosyltransferase 1 (galactoside 2-alpha-L-fucosyltransferase, H blood group)], FUT4 [fucosyltransferase 4 (alpha (1 [3) fucosyltransferase, myeloid-specific)], FXN [frataxin], FXR1 [fragile X mental retardation, autosomal homolog 1], FXR2 [fragile X mental retardation, autosomal homolog 2], FXYD1 [FXYD domain containing ion transport regulator 1], FYB [FYN binding protein (FYB-120/130)], FYN [FYN oncogene related to SRC, FGR, YES], FZD1 [frizzled homolog 1 (Drosophila)], FZD10 [frizzled homolog 10 (Drosophila)], FZD2 [frizzled homolog 2 (Drosophila)], FZD3 [frizzled homolog 3 (Drosophila)], FZD4 [frizzled homolog 4 (Drosophila)], FZD5 [frizzled homolog 5 (Drosophila)], FZD6 [frizzled homolog 6 (Drosophila)], FZD7 [frizzled homolog 7 (Drosophila)], FZD8 [frizzled homolog 8 (Drosophila)], FZD9 [frizzled homolog 9 (Drosophila)], FZR1 [fizzy/cell division cycle 20 related 1 (Drosophila)], G6PD [glucose-6-phosphate dehydrogenase], GAA [glucosidase, alpha; acid], GAB1 [GRB2-associated binding protein 1], GABARAP [GABA(A) receptor-associated protein], GABBR1 [gamma-aminobutyric acid (GABA) B receptor, 1], GABBR2 [gamma-aminobutyric acid (GABA) B receptor, 2], GABPA [GA binding protein transcription factor, alpha subunit 60 kDa], GABRA1 [gamma-aminobutyric acid (GABA) A receptor, alpha 1], GABRA2 [gamma-aminobutyric acid (GABA) A receptor, alpha 2], GABRA3 [gamma-aminobutyric acid (GABA) A receptor, alpha 3], GABRA4 [gamma-aminobutyric acid (GABA) A receptor, alpha 4], GABRA5 [gamma-aminobutyric acid (GABA) A receptor, alpha 5], GABRA6 [gamma-aminobutyric acid (GABA) A receptor, alpha 6], GABRB1 [gamma-aminobutyric acid (GABA) A receptor, beta 1], GABRB2 [gamma-aminobutyric acid (GABA) A receptor, beta 2], GABRB3 [gamma-aminobutyric acid (GABA) A receptor, beta 3], GABRD [gamma-aminobutyric acid (GABA) A receptor, delta], GABRE [gamma-aminobutyric acid (GABA) A receptor, epsilon], GABRG1 [gamma-aminobutyric acid (GABA) A receptor, gamma 1], GABRG2 [gamma-aminobutyric acid (GABA) A receptor, gamma 2], GABRG3 [gamma-aminobutyric acid (GABA) A receptor, gamma 3], GABRP [gamma-aminobutyric acid (GABA) A receptor, pi], GAD1 [glutamate decarboxylase 1 (brain, 67 kDa)], GAD2 [glutamate decarboxylase 2 (pancreatic islets and brain, 65 kDa)], GAL [galanin prepropeptide], GALE [UDP-galactose-4-epimerase], GALK1 [galactokinase 1], GALT [galactose-1-phosphate uridylyltransferase], GAP43 [growth associated protein 43], GAPDH [glyceraldehyde-3-phosphate dehydrogenase], GARS [glycyl-tRNA synthetase], GART [phosphoribosylglycinamide formyltransferase, phosphoribosylglycinamide synthetase, phosphoribosylaminoimidazole synthetase], GAS1 [growth arrest-specific 1], GAS6 [growth arrest-specific 6], GAST [gastrin], GATA1 [GATA binding protein 1 (globin transcription factor 1)], GATA2 [GATA binding protein 2], GATA3 [GATA binding protein 3], GATA4 [GATA binding protein 4], GATA6 [GATA binding protein 6], GBA [glucosidase, beta, acid], GBE1 [glucan (1 [4-alpha-), branching enzyme 1], GBX2 [gastrulation brain homeobox 2], GC [group-specific component (vitamin D binding protein)], GCG [glucagon], GCH1 [GTP cyclohydrolase 1], GCNT1 [glucosaminyl (N-acetyl) transferase 1, core 2], GDAP1 [ganglioside-induced differentiation-associated protein 1], GDF1 [growth differentiation factor 1], GDF11 [growth differentiation factor 11], GDF15 [growth differentiation factor 15], GDF7 [growth differentiation factor 7], GDI1 [GDP dissociation inhibitor 1], GDI2 [GDP dissociation inhibitor 2], GDNF [glial cell derived neurotrophic factor], GDPD5 [glycerophosphodiester phosphodiesterase domain containing 5], GEM [GTP binding protein overexpressed in skeletal muscle], GFAP [glial fibrillary acidic protein], GFER [growth factor, augmenter of liver regeneration], GFI1B [growth factor independent 1B transcription repressor], GFRA1 [GDNF family receptor alpha 1], GFRA2 [GDNF family receptor alpha 2], GFRA3 [GDNF family receptor alpha 3], GFRA4 [GDNF family receptor alpha 4], GGCX [gamma-glutamyl carboxylase], GGNBP2 [gametogenetin binding protein 2], GGT1 [gamma-glutamyltransferase 1], GGT2 [gamma-glutamyltransferase 2], GH1 [growth hormone 1], GHR [growth hormone receptor], GHRH [growth hormone releasing hormone], GHRHR [growth hormone releasing hormone receptor], GHRL [ghrelin/obestatin prepropeptide], GHSR [growth hormone secretagogue receptor], GIPR [gastric inhibitory polypeptide receptor], GIT1 [G protein-coupled receptor kinase interacting ArfGAP 1], GJA1 [gap junction protein, alpha 1, 43 kDa], GJA4 [gap junction protein, alpha 4, 37 kDa], GJA5 [gap junction protein, alpha 5, 40 kDa], GJB1 [gap junction protein, beta 1, 32 kDa], GJB2 [gap junction protein, beta 2, 26 kDa], GJB6 [gap junction protein, beta 6, 30 kDa], GLA [galactosidase, alpha], GLB1 [galactosidase, beta 1], GLDC [glycine dehydrogenase (decarboxylating)], GLI1 [GLI family zinc finger 1], GLI2 [GLI family zinc finger 2], GLI3 [GLI family zinc finger 3], GLIS1 [GLIS family zinc finger 1], GLIS2 [GLIS family zinc finger 2], GLO1 [glyoxalase I], GLRA2 [glycine receptor, alpha 2], GLRB [glycine receptor, beta], GLS [glutaminase], GLUD1 [glutamate dehydrogenase 1], GLUD2 [glutamate dehydrogenase 2], GLUL [glutamate-ammonia ligase (glutamine synthetase)], GLYAT [glycine-N-acyltransferase], GMFB [glia maturation factor, beta], GMNN [geminin, DNA replication inhibitor], GMPS [guanine monphosphate synthetase], GNA11 [guanine nucleotide binding protein (G protein), alpha 11 (Gq class)], GNA12 [guanine nucleotide binding protein (G protein) alpha 12], GNA13 [guanine nucleotide binding protein (G protein), alpha 13], GNA14 [guanine nucleotide binding protein (G protein), alpha 14], GNA15 [guanine nucleotide binding protein (G protein), alpha 15 (Gq class)], GNAI1 [guanine nucleotide binding protein (G protein), alpha inhibiting activity polypeptide 1], GNAI2 [guanine nucleotide binding protein (G protein), alpha inhibiting activity polypeptide 2], GNAI3 [guanine nucleotide binding protein (G protein), alpha inhibiting activity polypeptide 3], GNAL [guanine nucleotide binding protein (G protein), alpha activating activity polypeptide, olfactory type], GNAO1 [guanine nucleotide binding protein (G protein), alpha activating activity polypeptide 0], GNAQ [guanine nucleotide binding protein (G protein), q polypeptide], GNAS [GNAS complex locus], GNAT1 [guanine nucleotide binding protein (G protein), alpha transducing activity polypeptide 1], GNAT2 [guanine nucleotide binding protein (G protein), alpha transducing activity polypeptide 2], GNAZ [guanine nucleotide binding protein (G protein), alpha z polypeptide], GNB1 [guanine nucleotide binding protein (G protein), beta polypeptide 1], GNB1L [guanine nucleotide binding protein (G protein), beta polypeptide 1-like], GNB2 [guanine nucleotide binding protein (G protein), beta polypeptide 2], GNB2L1 [guanine nucleotide binding protein (G protein), beta polypeptide 2-like 1], GNB3 [guanine nucleotide binding protein (G protein), beta polypeptide 3], GNB4 [guanine nucleotide binding protein (G protein), beta polypeptide 4], GNB5 [guanine nucleotide binding protein (G protein), beta 5], GNG10 [guanine nucleotide binding protein (G protein), gamma 10], GNG11 [guanine nucleotide binding protein (G protein), gamma 11], GNG12 [guanine nucleotide binding protein (G protein), gamma 12], GNG13 [guanine nucleotide binding protein (G protein), gamma 13], GNG2 [guanine nucleotide binding protein (G protein), gamma 2], GNG3 [guanine nucleotide binding protein (G protein), gamma 3], GNG4 [guanine nucleotide binding protein (G protein), gamma 4], GNG5 [guanine nucleotide binding protein (G protein), gamma 5], GNG7 [guanine nucleotide binding protein (G protein), gamma 7], GNLY [granulysin], GNRH1 [gonadotropin-releasing hormone 1 (luteinizing-releasing hormone)], GNRHR [gonadotropin-releasing hormone receptor], GOLGA2 [golgin A2], GOLGA4 [golgin A4], GOT2 [glutamic-oxaloacetic transaminase 2, mitochondrial (aspartate aminotransferase 2)], GP1 BA [glycoprotein Ib (platelet), alpha polypeptide], GP5 [glycoprotein V (platelet)], GP6 [glycoprotein VI (platelet)], GP9 [glycoprotein IX (platelet)], GPC1 [glypican 1], GPC3 [glypican 3], GPD1 [glycerol-3-phosphate dehydrogenase 1 (soluble)], GPHN [gephyrin], GPI [glucose phosphate isomerase], GPM6A [glycoprotein M6A], GPM6B [glycoprotein M6B], GPR161 [G protein-coupled receptor 161], GPR182 [G protein-coupled receptor 182], GPR56 [G protein-coupled receptor 56], GPRC6A [G protein-coupled receptor, family C, group 6, member A], GPRIN1 [G protein regulated inducer of neurite outgrowth 1], GPT [glutamic-pyruvate transaminase (alanine aminotransferase)], GPT2 [glutamic pyruvate transaminase (alanine aminotransferase) 2], GPX1 [glutathione peroxidase 1], GPX3 [glutathione peroxidase 3 (plasma)], GPX4 [glutathione peroxidase 4 (phospholipid hydroperoxidase)], GRAP [GRB2-related adaptor protein], GRB10 [growth factor receptor-bound protein 10], GRB2 [growth factor receptor-bound protein 2], GRB7 [growth factor receptor-bound protein 7], GREM1 [gremlin 1, cysteine knot superfamily, homolog (Xenopus laevis)], GRIA1 [glutamate receptor, ionotropic, AMPA 1], GRIA2 [glutamate receptor, ionotropic, AMPA 2], GRIA3 [glutamate receptor, ionotrophic, AMPA 3], GRID2 [glutamate receptor, ionotropic, delta 2], GRID21P [glutamate receptor, ionotropic, delta 2 (Grid2) interacting protein], GRIK1 [glutamate receptor, ionotropic, kainate 1], GRIK2 [glutamate receptor, ionotropic, kainate 2], GRIN1 [glutamate receptor, ionotropic, N-methyl D-aspartate 1], GRIN2A [glutamate receptor, ionotropic, N-methyl D-aspartate 2A], GRIP1 [glutamate receptor interacting protein 1], GRLF1 [glucocorticoid receptor DNA binding factor 1], GRM1 [glutamate receptor, metabotropic 1], GRM2 [glutamate receptor, metabotropic 2], GRM5 [glutamate receptor, metabotropic 5], GRM7 [glutamate receptor, metabotropic 7], GRM8 [glutamate receptor, metabotropic 8], GRN [granulin], GRP [gastrin-releasing peptide], GRPR [gastrin-releasing peptide receptor], GSK3B [glycogen synthase kinase 3 beta], GSN [gelsolin], GSR [glutathione reductase], GSS [glutathione synthetase], GSTA1 [glutathione S-transferase alpha 1], GSTM1 [glutathione S-transferase mu 1], GSTP1 [glutathione S-transferase pi 1], GSTT1 [glutathione S-transferase theta 1], GSTZ1 [glutathione transferase zeta 1], GTF2B [general transcription factor IIB], GTF2E2 [general transcription factor 11E, polypeptide 2, beta 34 kDa], GTF2H1 [general transcription factor 11H, polypeptide 1, 62 kDa], GTF2H2 [general transcription factor 11H, polypeptide 2, 44 kDa], GTF2H3 [general transcription factor 11H, polypeptide 3, 34 kDa], GTF2H4 [general transcription factor 11H, polypeptide 4, 52 kDa], GTF2I [general transcription factor IIi], GTF21RD1 [GTF2I repeat domain containing 1], GTF21RD2 [GTF2I repeat domain containing 2], GUCA2A [guanylate cyclase activator 2A (guanylin)], GUCY1A3 [guanylate cyclase 1, soluble, alpha 3], GUSB [glucuronidase, beta], GYPA [glycophorin A (MNS blood group)], GYPC [glycophorin C (Gerbich blood group)], GZF1 [GDNF-inducible zinc finger protein 1], GZMA [granzyme A (granzyme 1, cytotoxic T-lymphocyte-associated serine esterase 3)], GZMB [granzyme B (granzyme 2, cytotoxic T-lymphocyte-associated serine esterase 1)], H19 [H19, imprinted maternally expressed transcript (non-protein coding)], H1F0 [H1 histone family, member 0], H2AFX [H2A histone family, member X], H2AFY [H2A histone family, member Y], H6PD [hexose-6-phosphate dehydrogenase (glucose 1-dehydrogenase)], HADHA [hydroxyacyl-Coenzyme A dehydrogenase/3-ketoacyl-Coenzyme A thiolase/enoyl-Coenzyme A hydratase (trifunctional protein), alpha subunit], HAMP [hepcidin antimicrobial peptide], HAND1 [heart and neural crest derivatives expressed 1], HAND2 [heart and neural crest derivatives expressed 2], HAP1 [huntingtin-associated protein 1], HAPLN1 [hyaluronan and proteoglycan link protein 1], HARS [histidyl-tRNA synthetase], HAS1 [hyaluronan synthase 1], HAS2 [hyaluronan synthase 2], HAS3 [hyaluronan synthase 3], HAX1 [HCLS1 associated protein X-1], HBA2 [hemoglobin, alpha 2], HBB [hemoglobin, beta], HBEGF [heparin-binding EGF-like growth factor], HBG1 [hemoglobin, gamma A], HBG2 [hemoglobin, gamma G], HCCS [holocytochrome c synthase (cytochrome c heme-lyase)], HCK [hemopoietic cell kinase], HCLS1 [hematopoietic cell-specific Lyn substrate 1], HCN4 [hyperpolarization activated cyclic nucleotide-gated potassium channel 4], HCRT [hypocretin (orexin) neuropeptide precursor], HCRTR1 [hypocretin (orexin) receptor 1], HCRTR2 [hypocretin (orexin) receptor 2], HDAC1 [histone deacetylase 1], HDAC2 [histone deacetylase 2], HDAC4 [histone deacetylase 4], HDAC9 [histone deacetylase 9], HDC [histidine decarboxylase], HDLBP [high density lipoprotein binding protein], HEPACAM [hepatocyte cell adhesion molecule], HES1 [hairy and enhancer of split 1, (Drosophila)], HES3 [hairy and enhancer of split 3 (Drosophila)], HES5 [hairy and enhancer of split 5 (Drosophila)], HES6 [hairy and enhancer of split 6 (Drosophila)], HEXA [hexosaminidase A (alpha polypeptide)], HFE [hemochromatosis], HFE2 [hemochromatosis type 2 (juvenile)], HGF [hepatocyte growth factor (hepapoietin A; scatter factor)], HGS [hepatocyte growth factor-regulated tyrosine kinase substrate], HHEX [hematopoietically expressed homeobox], HHIP [hedgehog interacting protein], HIF1A [hypoxia inducible factor 1, alpha subunit (basic helix-loop-helix transcription factor)], HINT1 [histidine triad nucleotide binding protein 1], HIPK2 [homeodomain interacting protein kinase 2], HIRA [HIR histone cell cycle regulation defective homolog A (S. cerevisiae)], HIRIP3 [HIRA interacting protein 3], H1ST1H2AB [histone cluster 1, H2ab], H1ST1H2AC [histone cluster 1, H2ac], H1ST1H2AD [histone cluster 1, H2ad], H1ST1H2AE [histone cluster 1, H2ae], H1ST1H2AG [histone cluster 1, H2ag], H1ST1H2AI [histone cluster 1, H2ai], H1ST1H2AJ [histone cluster 1, H2aj], H1ST1H2AK [histone cluster 1, H2ak], H1ST1H2AL [histone cluster 1, H2al], H1ST1H2AM [histone cluster 1, H2 am], HIST1H3E [histone cluster 1, H3e], H1ST2H2AA3 [histone cluster 2, H2aa3], H1ST2H2AA4 [histone cluster 2, H2aa4], H1ST2H2AC [histone cluster 2, H2ac], HKR1 [GLI-Kruppel family member HKR1], HLA-A [major histocompatibility complex, class I, A], HLA-B [major histocompatibility complex, class I, B], HLA-C [major histocompatibility complex, class I, C], HLA-DMA [major histocompatibility complex, class II, DM alpha], HLA-DOB [major histocompatibility complex, class II, DO beta], HLA-DQA1 [major histocompatibility complex, class II, DQ alpha 1], HLA-DQB1 [major histocompatibility complex, class II, DQ beta 1], HLA-DRA [major histocompatibility complex, class II, DR alpha], HLA-DRB1 [major histocompatibility complex, class II, DR beta 1], HLA-DRB4 [major histocompatibility complex, class II, DR beta 4], HLA-DRB5 [major histocompatibility complex, class II, DR beta 5], HLA-E [major histocompatibility complex, class I, E], HLA-F [major histocompatibility complex, class I, F], HLA-G [major histocompatibility complex, class I, G], HLCS [holocarboxylase synthetase (biotin-(proprionyl-Coenzyme A-carboxylase (ATP-hydrolysing)) ligase)], HMBS [hydroxymethylbilane synthase], HMGA1 [high mobility group AT-hook 1], HMGA2 [high mobility group AT-hook 2], HMGB1 [high-mobility group box 1], HMGCR [3-hydroxy-3-methylglutaryl-Coenzyme A reductase], HMGN1 [high-mobility group nucleosome binding domain 1], HMOX1 [heme oxygenase (decycling) 1], HMOX2 [heme oxygenase (decycling) 2], HNF1A [HNF1 homeobox A], HNF4A [hepatocyte nuclear factor 4, alpha], HNMT [histamine N-methyltransferase], HNRNPA2B1 [heterogeneous nuclear ribonucleoprotein A2/B1], HNRNPK [heterogeneous nuclear ribonucleoprotein K], HNRNPL [heterogeneous nuclear ribonucleoprotein L], HNRNPU [heterogeneous nuclear ribonucleoprotein U (scaffold attachment factor A)], HNRPDL [heterogeneous nuclear ribonucleoprotein D-like], HOMER1 [homer homolog 1 (Drosophila)], HOXA1 [homeobox A1], HOXA10 [homeobox A10], HOXA2 [homeobox A2], HOXA5 [homeobox A5], HOXA9 [homeobox A9], HOXB1 [homeobox B1], HOXB4 [homeobox B4], HOXB9 [homeobox B9], HOXD11 [homeobox D11], HOXD12 [homeobox D12], HOXD13 [homeobox D13], HP [haptoglobin], HPD [4-hydroxyphenylpyruvate dioxygenase], HPRT1 [hypoxanthine phosphoribosyltransferase 1], HPS4 [Hermansky-Pudlak syndrome 4], HPX [hemopexin], HRAS [v-Ha-ras Harvey rat sarcoma viral oncogene homolog], HRG [histidine-rich glycoprotein], HRH1 [histamine receptor H1], HRH2 [histamine receptor H2], HRH3 [histamine receptor H3], HSD11B1 [hydroxysteroid (11-beta) dehydrogenase 1], HSD11B2 [hydroxysteroid (11-beta) dehydrogenase 2], HSD17B10 [hydroxysteroid (17-beta) dehydrogenase 10], HSD3B2 [hydroxy-delta-5-steroid dehydrogenase, 3 beta- and steroid delta-isomerase 2], HSF1 [heat shock transcription factor 1], HSP90AA1 [heat shock protein 90 kDa alpha (cytosolic), class A member 1], HSP90B1 [heat shock protein 90 kDa beta (Grp94), member 1], HSPA1A [heat shock 70 kDa protein 1A], HSPA4 [heat shock 70 kDa protein 4], HSPA5 [heat shock 70 kDa protein 5 (glucose-regulated protein, 78 kDa)], HSPA8 [heat shock 70 kDa protein 8], HSPA9 [heat shock 70 kDa protein 9 (mortalin)], HSPB1 [heat shock 27 kDa protein 1], HSPD1 [heat shock 60 kDa protein 1 (chaperonin)], HSPE1 [heat shock 10 kDa protein 1 (chaperonin 10)], HSPG2 [heparan sulfate proteoglycan 2], HTN1 [histatin 1], HTR1A [5-hydroxytryptamine (serotonin) receptor 1A], HTR1B [5-hydroxytryptamine (serotonin) receptor 1 B], HTR1D [5-hydroxytryptamine (serotonin) receptor 1 D], HTR1E [5-hydroxytryptamine (serotonin) receptor 1 E], HTR1F [5-hydroxytryptamine (serotonin) receptor 1F], HTR2A [5-hydroxytryptamine (serotonin) receptor 2A], HTR2B [5-hydroxytryptamine (serotonin) receptor 2B], HTR2c [5-hydroxytryptamine (serotonin) receptor 20], HTR3A [5-hydroxytryptamine (serotonin) receptor 3A], HTR3B [5-hydroxytryptamine (serotonin) receptor 3B], HTR5A [5-hydroxytryptamine (serotonin) receptor 5A], HTR6 [5-hydroxytryptamine (serotonin) receptor 6], HTR7 [5-hydroxytryptamine (serotonin) receptor 7 (adenylate cyclase-coupled)], HTT [huntingtin], HYAL1 [hyaluronoglucosaminidase 1], HYOU1 [hypoxia up-regulated 1], IAPP [islet amyloid polypeptide], IBSP [integrin-binding sialoprotein], ICAM1 [intercellular adhesion molecule 1], ICAM2 [intercellular adhesion molecule 2], ICAM3 [intercellular adhesion molecule 3], ICAM5 [intercellular adhesion molecule 5, telencephalin], ICOS [inducible T-cell co-stimulator], ID1 [inhibitor of DNA binding 1, dominant negative helix-loop-helix protein], ID2 [inhibitor of DNA binding 2, dominant negative helix-loop-helix protein], ID3 [inhibitor of DNA binding 3, dominant negative helix-loop-helix protein], ID4 [inhibitor of DNA binding 4, dominant negative helix-loop-helix protein], IDE [insulin-degrading enzyme], IDI1 [isopentenyl-diphosphate delta isomerase 1], ID01 [indoleamine 2 [3-dioxygenase 1], IDS [iduronate 2-sulfatase], IDUA [iduronidase, alpha-L-], IER3 [immediate early response 3], IF127 [interferon, alpha-inducible protein 27], IFNA1 [interferon, alpha 1], IFNA2 [interferon, alpha 2], IFNAR1 [interferon (alpha, beta and omega) receptor 1], IFNAR2 [interferon (alpha, beta and omega) receptor 2], IFNB1 [interferon, beta 1, fibroblast], IFNG [interferon, gamma], IFNGR1 [interferon gamma receptor 1], IFNGR2 [interferon gamma receptor 2 (interferon gamma transducer 1)], IGF1 [insulin-like growth factor 1 (somatomedin C)], IGF1 R [insulin-like growth factor 1 receptor], IGF2 [insulin-like growth factor 2 (somatomedin A)], IGF2R [insulin-like growth factor 2 receptor], IGFBP1 [insulin-like growth factor binding protein 1], IGFBP2 [insulin-like growth factor binding protein 2, 36 kDa], IGFBP3 [insulin-like growth factor binding protein 3], IGFBP4 [insulin-like growth factor binding protein 4], IGFBP5 [insulin-like growth factor binding protein 5], IGFBP6 [insulin-like growth factor binding protein 6], IGFBP7 [insulin-like growth factor binding protein 7], IGHA1 [immunoglobulin heavy constant alpha 1], IGHE [immunoglobulin heavy constant epsilon], IGHG1 [immunoglobulin heavy constant gamma 1 (G1 m marker)], IGHJ1 [immunoglobulin heavy joining 1], IGHM [immunoglobulin heavy constant mu], IGHMBP2 [immunoglobulin mu binding protein 2], IGKC [immunoglobulin kappa constant], IKBKAP [inhibitor of kappa light polypeptide gene enhancer in B-cells, kinase complex-associated protein], IKBKB [inhibitor of kappa light polypeptide gene enhancer in B-cells, kinase beta], IKZF1 [IKAROS family zinc finger 1 (Ikaros)], IL10 [interleukin 10], IL10RA [interleukin 10 receptor, alpha], IL10RB [interleukin 10 receptor, beta], IL11 [interleukin 11], IL11RA [interleukin 11 receptor, alpha], IL12A [interleukin 12A (natural killer cell stimulatory factor 1, cytotoxic lymphocyte maturation factor 1, p35)], IL12B [interleukin 12B (natural killer cell stimulatory factor 2, cytotoxic lymphocyte maturation factor 2, p40)], IL12RB1 [interleukin 12 receptor, beta 1], IL13 [interleukin 13], IL15 [interleukin 15], IL15RA [interleukin 15 receptor, alpha], IL16 [interleukin 16 (lymphocyte chemoattractant factor)], IL17A [interleukin 17A], IL18 [interleukin 18 (interferon-gamma-inducing factor)], IL18BP [interleukin 18 binding protein], IL1A [interleukin 1, alpha], IL1B [interleukin 1, beta], IL1F7 [interleukin 1 family, member 7 (zeta)], IL1R1 [interleukin 1 receptor, type I], IL1 R2 [interleukin 1 receptor, type II], IL1 RAPL1 [interleukin 1 receptor accessory protein-like 1], IL1 RL1 [interleukin 1 receptor-like 1], URN [interleukin 1 receptor antagonist], IL2 [interleukin 2], IL21 [interleukin 21], IL22 [interleukin 22], IL23A [interleukin 23, alpha subunit p19], IL23R [interleukin 23 receptor], IL29 [interleukin 29 (interferon, lambda 1)], IL2RA [interleukin 2 receptor, alpha], IL2RB [interleukin 2 receptor, beta], IL3 [interleukin 3 (colony-stimulating factor, multiple)], IL3RA [interleukin 3 receptor, alpha (low affinity)], IL4 [interleukin 4], IL4R [interleukin 4 receptor], IL5 [interleukin 5 (colony-stimulating factor, eosinophil)], IL6 [interleukin 6 (interferon, beta 2)], IL6R [interleukin 6 receptor], IL6ST [interleukin 6 signal transducer (gp130, oncostatin M receptor)], IL7 [interleukin 7], IL7R [interleukin 7 receptor], IL8 [interleukin 8], IL9 [interleukin 9], ILK [integrin-linked kinase], IMMP2L [IMP2 inner mitochondrial membrane peptidase-like (S. cerevisiae)], IMMT [inner membrane protein, mitochondrial (mitofilin)], IMPA1 [inositol(myo)-[(or 4)-monophosphatase 1], IMPDH2 [IMP (inosine monophosphate) dehydrogenase 2], INADL [InaD-like (Drosophila)], INCENP [inner centromere protein antigens 135/155 kDa], ING1 [inhibitor of growth family, member 1], ING3 [inhibitor of growth family, member 3], INHA [inhibin, alpha], INHBA [inhibin, beta A], INPP1 [inositol polyphosphate-1-phosphatase], INPP5D [inositol polyphosphate-5-phosphatase, 145 kDa], INPP5E [inositol polyphosphate-5-phosphatase, 72 kDa], INPP5J [inositol polyphosphate-5-phosphatase J], INPPL1 [inositol polyphosphate phosphatase-like 1], INS [insulin], INSIG2 [insulin induced gene 2], INS-IGF2 [INS-IGF2 readthrough transcript], INSL3 [insulin-like 3 (Leydig cell)], INSR [insulin receptor], INVS [inversin], IQCB1 [IQ motif containing B1], IQGAP1 [IQ motif containing GTPase activating protein 1], IRAK1 [interleukin-1 receptor-associated kinase 1], IRAK4 [interleukin-1 receptor-associated kinase 4], IREB2 [iron-responsive element binding protein 2], IRF1 [interferon regulatory factor 1], IRF4 [interferon regulatory factor 4], IRF8 [interferon regulatory factor 8], IRS1 [insulin receptor substrate 1], IRS2 [insulin receptor substrate 2], IRS4 [insulin receptor substrate 4], IRX3 [iroquois homeobox 3], ISG15 [ISG15 ubiquitin-like modifier], ISL1 [ISL L1M homeobox 1], ISL2 [ISL LIM homeobox 2], ISLR2 [immunoglobulin superfamily containing leucine-rich repeat 2], ITGA2 [integrin, alpha 2 (CD49B, alpha 2 subunit of VLA-2 receptor)], ITGA2B [integrin, alpha 2b (platelet glycoprotein IIb of IIb/IIIa complex, antigen CD41)], ITGA3 [integrin, alpha 3 (antigen CD49C, alpha 3 subunit of VLA-3 receptor)], ITGA4 [integrin, alpha 4 (antigen CD49D, alpha 4 subunit of VLA-4 receptor)], ITGA5 [integrin, alpha 5 (fibronectin receptor, alpha polypeptide)], ITGA6 [integrin, alpha 6], ITGA9 [integrin, alpha 9], ITGAL [integrin, alpha L (antigen CD11A (p180), lymphocyte function-associated antigen 1; alpha polypeptide)], ITGAM [integrin, alpha M (complement component 3 receptor 3 subunit)], ITGAV [integrin, alpha V (vitronectin receptor, alpha polypeptide, antigen CD51)], ITGAX [integrin, alpha X (complement component 3 receptor 4 subunit)], ITGB1 [integrin, beta 1 (fibronectin receptor, beta polypeptide, antigen CD29 includes MDF2, MSK12)], ITGB2 [integrin, beta 2 (complement component 3 receptor 3 and 4 subunit)], ITGB3 [integrin, beta 3 (platelet glycoprotein 111a, antigen CD61)], ITGB4 [integrin, beta 4], ITGB6 [integrin, beta 6], ITGB7 [integrin, beta 7], ITIH4 [inter-alpha (globulin) inhibitor H4 (plasma Kallikrein-sensitive glycoprotein)], ITM2B [integral membrane protein 2B], ITPR1 [inositol 1 [4 Ξ-triphosphate receptor, type 1], ITPR2 [inositol 1 [4 Ξ-triphosphate receptor, type 2], ITPR3 [inositol 1 [4 Ξ-triphosphate receptor, type 3], ITSN1 [intersectin 1 (SH3 domain protein)], ITSN2 [intersectin 2], IVL [involucrin], JAG1 [jagged 1 (Alagille syndrome)], JAK1 [Janus kinase 1], JAK2 [Janus kinase 2], JAK3 [Janus kinase 3], JAM2 [junctional adhesion molecule 2], JARID2 [jumonji, AT rich interactive domain 2], JMJD1C [jumonji domain containing 10], JMY [junction mediating and regulatory protein, p53 cofactor], JRKL [jerky homolog-like (mouse)], JUN [jun oncogene], JUNB [jun B proto-oncogene], JUND [jun D proto-oncogene], JUP [junction plakoglobin], KAL1 [Kallmann syndrome 1 sequence], KALRN [kalirin, RhoGEF kinase], KARS [lysyl-tRNA synthetase], KAT2B [K(lysine) acetyltransferase 2B], KATNA1 [katanin p60 (ATPase-containing) subunit A 1], KATNB1 [katanin p80 (WD repeat containing) subunit B1], KCNA4 [potassium voltage-gated channel, shaker-related subfamily, member 4], KCND1 [potassium voltage-gated channel, ShaI-related subfamily, member 1], KCND2 [potassium voltage-gated channel, ShaI-related subfamily, member 2], KCNE1 [potassium voltage-gated channel, Isk-related family, member 1], KCNE2 [potassium voltage-gated channel, Isk-related family, member 2], KCNH₂ [potassium voltage-gated channel, subfamily H (eag-related), member 2], KCNH₄ [potassium voltage-gated channel, subfamily H (eag-related), member 4], KCNJ15 [potassium inwardly-rectifying channel, subfamily J, member 15], KCNJ3 [potassium inwardly-rectifying channel, subfamily J, member 3], KCNJ4 [potassium inwardly-rectifying channel, subfamily J, member 4], KCNJ5 [potassium inwardly-rectifying channel, subfamily J, member 5], KCNJ6 [potassium inwardly-rectifying channel, subfamily J, member 6], KCNMA1 [potassium large conductance calcium-activated channel, subfamily M, alpha member 1], KCNN1 [potassium intermediate/small conductance calcium-activated channel, subfamily N, member 1], KCNN2 [potassium intermediate/small conductance calcium-activated channel, subfamily N, member 2], KCNN3 [potassium intermediate/small conductance calcium-activated channel, subfamily N, member 3], KCNQ1 [potassium voltage-gated channel, KQT-like subfamily, member 1], KCNQ2 [potassium voltage-gated channel, KQT-like subfamily, member 2], KDM5C [lysine (K)-specific demethylase 5C], KDR [kinase insert domain receptor (a type III receptor tyrosine kinase)], KIAA0101 [KIAA0101], KIAA0319 [KIAA0319], KIAA1715 [KIAA1715], KIDINS220 [kinase D-interacting substrate, 220 kDa], KIF15 [kinesin family member 15], KIF16B [kinesin family member 16B], KIF1A [kinesin family member 1A], KIF2A [kinesin heavy chain member 2A], KIF2B [kinesin family member 2B], KIF3A [kinesin family member 3A], KIF5C [kinesin family member 5C], KIF7 [kinesin family member 7], KIR2□L1 [killer cell immunoglobulin-like receptor, two domains, long cytoplasmic tail, 1], KIR2□L3 [killer cell immunoglobulin-like receptor, two domains, long cytoplasmic tail, 3], KIR2DS2 [killer cell immunoglobulin-like receptor, two domains, short cytoplasmic tail, 2], KIR3□L1 [killer cell immunoglobulin-like receptor, three domains, long cytoplasmic tail, 1], KIR3□L2 [killer cell immunoglobulin-like receptor, three domains, long cytoplasmic tail, 2], KIRREL3 [kin of IRRE like 3 (Drosophila)], KISS1 [KiSS-1 metastasis-suppressor], KISS1 R [KISS1 receptor], KIT [v-kit Hardy-Zuckerman 4 feline sarcoma viral oncogene homolog], KITLG [KIT ligand], KL [klotho], KLF7 [Kruppel-like factor 7 (ubiquitous)], KLK1 [kallikrein 1], KLK10 [kallikrein-related peptidase 10], KLK11 [kallikrein-related peptidase 11], KLK2 [kallikrein-related peptidase 2], KLK3 [kallikrein-related peptidase 3], KLK5 [kallikrein-related peptidase 5], KLRD1 [killer cell lectin-like receptor subfamily D, member 1], KLRK1 [killer cell lectin-like receptor subfamily K, member 1], KMO [kynurenine 3-monooxygenase (kynurenine 3-hydroxylase)], KNG1 [kininogen 1], KPNA2 [karyopherin alpha 2 (RAG cohort 1, importin alpha 1)], KPNB1 [karyopherin (importin) beta 1], KPTN [kaptin (actin binding protein)], KRAS [v-Ki-ras2 Kirsten rat sarcoma viral oncogene homolog], KRIT1 [KRIT1, ankyrin repeat containing], KRT1 [keratin 1], KRT10 [keratin 10], KRT14 [keratin 14], KRT18 [keratin 18], KRT19 [keratin 19], KRT3 [keratin 3], KRT5 [keratin 5], KRT7 [keratin 7], KRT8 [keratin 8], KRTAP19-3 [keratin associated protein 19-3], KRTAP2-1 [keratin associated protein 2-1], L1 CAM [L1 cell adhesion molecule], LACTB [lactamase, beta], LALBA [lactalbumin, alpha-], LAMA1 [laminin, alpha 1], LAMB1 [laminin, beta 1], LAMB2 [laminin, beta 2 (laminin S)], LAMB4 [laminin, beta 4], LAMP1 [lysosomal-associated membrane protein 1], LAMP2 [lysosomal-associated membrane protein 2], LAP3 [leucine aminopeptidase 3], LAPTM4A [lysosomal protein transmembrane 4 alpha], LARGE [like-glycosyltransferase], LARS [leucyl-tRNA synthetase], LASP1 [LIM and SH3 protein 1], LAT2 [linker for activation of T cells family, member 2], LBP [lipopolysaccharide binding protein], LBR [lamin B receptor], LCA10 [lung carcinoma-associated protein 10], LCA5 [Leber congenital amaurosis 5], LCAT [lecithin-cholesterol acyltransferase], LCK [lymphocyte-specific protein tyrosine kinase], LCN1 [lipocalin 1 (tear prealbumin)], LCN2 [lipocalin 2], LCP1 [lymphocyte cytosolic protein 1 (L-plastin)], LCP2 [lymphocyte cytosolic protein 2 (SH2 domain containing leukocyte protein of 76 kDa)], LCT [lactase], LDB1 [LIM domain binding 1], LDB2 [LIM domain binding 2], LDHA [lactate dehydrogenase A], LDLR [low density lipoprotein receptor], LDLRAP1 [low density lipoprotein receptor adaptor protein 1], LEF1 [lymphoid enhancer-binding factor 1], LEO1 [Leo1, Paf1/RNA polymerase II complex component, homolog (S. cerevisiae)], LEP [leptin], LEPR [leptin receptor], LGALS13 [lectin, galactoside-binding, soluble, 13], LGALS3 [lectin, galactoside-binding, soluble, 3], LGMN [legumain], LGR4 [leucine-rich repeat-containing G protein-coupled receptor 4], LGTN [ligatin], LHCGR [luteinizing hormone/choriogonadotropin receptor], LHFPL3 [lipoma HMGIC fusion partner-like 3], LHX1 [LIM homeobox 1], LHX2 [LIM homeobox 2], LHX3 [LIM homeobox 3], LHX4 [LIM homeobox 4], LHX9 [LIM homeobox 9], LIF [leukemia inhibitory factor (cholinergic differentiation factor)], LIFR [leukemia inhibitory factor receptor alpha], LIG1 [ligase I, DNA, ATP-dependent], LIG3 [ligase III, DNA, ATP-dependent], LIG4 [ligase IV, DNA, ATP-dependent], LILRA3 [leukocyte immunoglobulin-like receptor, subfamily A (without TM domain), member 3], LILRB1 [leukocyte immunoglobulin-like receptor, subfamily B (with TM and ITIM domains), member 1], LIMK1 [LIM domain kinase 1], LIMK2 [LIM domain kinase 2], LIN7A [lin-7 homolog A (C. elegans)], LIN7B [lin-7 homolog B (C. elegans)], LIN7C [lin-7 homolog C(C. elegans)], LINGO1 [leucine rich repeat and Ig domain containing 1], LIPC [lipase, hepatic], LIPE [lipase, hormone-sensitive], LLGL1 [lethal giant larvae homolog 1 (Drosophila)], LMAN1 [lectin, mannose-binding, 1], LMNA [lamin NC], LMO2 [LIM domain only 2 (rhombotin-like 1)], LMX1A [LIM homeobox transcription factor 1, alpha], LMX1 B [LIM homeobox transcription factor 1, beta], LNPEP [leucyl/cystinyl aminopeptidase], LOC400590 [hypothetical LOC400590], LOC646021 [similar to hCG1774990], LOC646030 [similar to hCG1991475], LOC646627 [phospholipase inhibitor], LOR [loricrin], LOX [lysyl oxidase], LOXL1 [lysyl oxidase-like 1], LPA [lipoprotein, Lp(a)], LPL [lipoprotein lipase], LPO [lactoperoxidase], LPP [LIM domain containing preferred translocation partner in lipoma], LPPR1 [lipid phosphate phosphatase-related protein type 1], LPPR3 [lipid phosphate phosphatase-related protein type 3], LPPR4 [lipid phosphate phosphatase-related protein type 4], LPXN [leupaxin], LRP1 [low density lipoprotein receptor-related protein 1], LRP6 [low density lipoprotein receptor-related protein 6], LRP8 [low density lipoprotein receptor-related protein 8, apolipoprotein e receptor], LRPAP1 [low density lipoprotein receptor-related protein associated protein 1], LRPPRC [leucine-rich PPR-motif containing], LRRC37B [leucine rich repeat containing 37B], LRRC4C [leucine rich repeat containing 4C], LRRTM1 [leucine rich repeat transmembrane neuronal 1], LSAMP [limbic system-associated membrane protein], LSM2 [LSM2 homolog, U6 small nuclear RNA associated (S. cerevisiae)], LSS [lanosterol synthase (2 [3-oxidosqualene-lanosterol cyclase)], LTA [lymphotoxin alpha (TNF superfamily, member 1)], LTA4H [leukotriene A4 hydrolase], LTBP1 [latent transforming growth factor beta binding protein 1], LTBP4 [latent transforming growth factor beta binding protein 4], LTBR [lymphotoxin beta receptor (TNFR superfamily, member 3)], LTC4S [leukotriene C4 synthase], LTF [lactotransferrin], LY96 [lymphocyte antigen 96], LYN [v-yes-1 Yamaguchi sarcoma viral related oncogene homolog], LYVE1 [lymphatic vessel endothelial hyaluronan receptor 1], M6PR [mannose-6-phosphate receptor (cation dependent)], MAB21L1 [mab-2′-like 1 (C. elegans)], MAB21L2 [mab-2′-like 2 (C. elegans)], MAF [v-maf musculoaponeurotic fibrosarcoma oncogene homolog (avian)], MAG [myelin associated glycoprotein], MAGEA1 [melanoma antigen family A, 1 (directs expression of antigen MZ2-E)], MAGEL2 [MAGE-like 2], MAL [mal, T-cell differentiation protein], MAML2 [mastermind-like 2 (Drosophila)], MAN2A1 [mannosidase, alpha, class 2A, member 1], MANBA [mannosidase, beta A, lysosomal], MANF [mesencephalic astrocyte-derived neurotrophic factor], MAOA [monoamine oxidase A], MAOB [monoamine oxidase B], MAP1 B [microtubule-associated protein 1 B], MAP2 [microtubule-associated protein 2], MAP2K1 [mitogen-activated protein kinase kinase 1], MAP2K2 [mitogen-activated protein kinase kinase 2], MAP2K3 [mitogen-activated protein kinase kinase 3], MAP2K4 [mitogen-activated protein kinase kinase 4], MAP3K1 [mitogen-activated protein kinase kinase kinase 1], MAP3K12 [mitogen-activated protein kinase kinase kinase 12], MAP3K13 [mitogen-activated protein kinase kinase kinase 13], MAP3K14 [mitogen-activated protein kinase kinase kinase 14], MAP3K4 [mitogen-activated protein kinase kinase kinase 4], MAP3K7 [mitogen-activated protein kinase kinase kinase 7], MAPK1 [mitogen-activated protein kinase 1], MAPK10 [mitogen-activated protein kinase 10], MAPK14 [mitogen-activated protein kinase 14], MAPK3 [mitogen-activated protein kinase 3], MAPK8 [mitogen-activated protein kinase 8], MAPK81P2 [mitogen-activated protein kinase 8 interacting protein 2], MAPK81P3 [mitogen-activated protein kinase 8 interacting protein 3], MAPK9 [mitogen-activated protein kinase 9], MAPKAPK2 [mitogen-activated protein kinase-activated protein kinase 2], MAPKSP1 [MAPK scaffold protein 1], MAPRE3 [microtubule-associated protein, RP/EB family, member 3], MAPT [microtubule-associated protein tau], MARCKS [myristoylated alanine-rich protein kinase C substrate], MARK1 [MAP/microtubule affinity-regulating kinase 1], MARK2 [MAP/microtubule affinity-regulating kinase 2], MAT2A [methionine adenosyltransferase II, alpha], MATR3 [matrin 3], MAX [MYC associated factor X], MAZ [MYC-associated zinc finger protein (purine-binding transcription factor)], MB [myoglobin], MBD1 [methyl-CpG binding domain protein 1], MBD2 [methyl-CpG binding domain protein 2], MBD3 [methyl-CpG binding domain protein 3], MBD4 [methyl-CpG binding domain protein 4], MBL2 [mannose-binding lectin (protein C) 2, soluble (opsonic defect)], MBP [myelin basic protein], MBTPS1 [membrane-bound transcription factor peptidase, site 1], MC1R [melanocortin 1 receptor (alpha melanocyte stimulating hormone receptor)], MC3R [melanocortin 3 receptor], MC4R [melanocortin 4 receptor], MCCC2 [methylcrotonoyl-Coenzyme A carboxylase 2 (beta)], MCF2L [MCF.2 cell line derived transforming sequence-like], MCHR1 [melanin-concentrating hormone receptor 1], MCL1 [myeloid cell leukemia sequence 1 (BCL2-related)], MCM7 [minichromosome maintenance complex component 7], MCPH1 [microcephalin 1], MDC1 [mediator of DNA-damage checkpoint 1], MDFIC [MyoD family inhibitor domain containing], MDGA1 [MAM domain containing glycosylphosphatidylinositol anchor 1], MDK [midkine (neurite growth-promoting factor 2)], MDM2 [Mdm2 p53 binding protein homolog (mouse)], ME2 [malic enzyme 2, NAD(+)-dependent, mitochondrial], MECP2 [methyl CpG binding protein 2 (Rett syndrome)], MED1 [mediator complex subunit 1], MED12 [mediator complex subunit 12], MED24 [mediator complex subunit 24], MEF2A [myocyte enhancer factor 2A], MEF2C [myocyte enhancer factor 20], MEIS1 [Meis homeobox 1], MEN1 [multiple endocrine neoplasia I], MERTK [c-mer proto-oncogene tyrosine kinase], MESP2 [mesoderm posterior 2 homolog (mouse)], MEST [mesoderm specific transcript homolog (mouse)], MET [met proto-oncogene (hepatocyte growth factor receptor)], METAP2 [methionyl aminopeptidase 2], METRN [meteorin, glial cell differentiation regulator], MFSD6 [major facilitator superfamily domain containing 6], MGAT2 [mannosyl (alpha-1 [6-)-glycoprotein beta-1 [2-N-acetylglucosaminyltransferase], MGMT [O-6-methylguanine-DNA methyltransferase], MGP [matrix Gla protein], MGST1 [microsomal glutathione S-transferase 1], MICA [MHC class I polypeptide-related sequence A], MICAL1 [microtubule associated monoxygenase, calponin and LIM domain containing 1], MICB [MHC class I polypeptide-related sequence B], MIF [macrophage migration inhibitory factor (glycosylation-inhibiting factor)], MITF [microphthalmia-associated transcription factor], MKI67 [antigen identified by monoclonal antibody Ki-67], MKKS [McKusick-Kaufman syndrome], MKNK1 [MAP kinase interacting serine/threonine kinase 1], MKRN3 [makorin ring finger protein 3], MKS1 [Meckel syndrome, type 1], MLH1 [mutL homolog 1, colon cancer, nonpolyposis type 2 (E. coli)], MLL [myeloid/lymphoid or mixed-lineage leukemia (trithorax homolog, Drosophila)], MLLT4 [myeloid/lymphoid or mixed-lineage leukemia (trithorax homolog, Drosophila); translocated to, 4], MLPH [melanophilin], MLX [MAX-like protein X], MLXIPL [MLX interacting protein-like], MME [membrane metallo-endopeptidase], MMP1 [matrix metallopeptidase 1 (interstitial collagenase)], MMP10 [matrix metallopeptidase 10 (stromelysin 2)], MMP12 [matrix metallopeptidase 12 (macrophage elastase)], MMP13 [matrix metallopeptidase 13 (collagenase 3)], MMP14 [matrix metallopeptidase 14 (membrane-inserted)], MMP2 [matrix metallopeptidase 2 (gelatinase A, 72 kDa gelatinase, 72 kDa type IV collagenase)], MMP24 [matrix metallopeptidase 24 (membrane-inserted)], MMP26 [matrix metallopeptidase 26], MMP3 [matrix metallopeptidase 3 (stromelysin 1, progelatinase)], MMP7 [matrix metallopeptidase 7 (matrilysin, uterine)], MMP8 [matrix metallopeptidase 8 (neutrophil collagenase)], MMP9 [matrix metallopeptidase 9 (gelatinase B, 92 kDa gelatinase, 92 kDa type IV collagenase)], MN1 [meningioma (disrupted in balanced translocation) 1], MNAT1 [menage a trois homolog 1, cyclin H assembly factor (Xenopus laevis)], MNX1 [motor neuron and pancreas homeobox 1], MOG [myelin oligodendrocyte glycoprotein], MPL [myeloproliferative leukemia virus oncogene], MPO [myeloperoxidase], MPP1 [membrane protein, palmitoylated 1, 55 kDa], MPZL1 [myelin protein zero-like 1], MR1 [major histocompatibility complex, class I-related], MRAP [melanocortin 2 receptor accessory protein], MRAS [muscle RAS oncogene homolog], MRC1 [mannose receptor, C type 1], MRGPRX1 [MAS-related GPR, member X1], MS4A1 [membrane-spanning 4-domains, subfamily A, member 1], MSH2 [mutS homolog 2, colon cancer, nonpolyposis type 1 (E. coli)], MSH3 [mutS homolog 3 (E. coli)], MSI1 [musashi homolog 1 (Drosophila)], MSN [moesin], MSR1 [macrophage scavenger receptor 1], MSTN [myostatin], MSX1 [msh homeobox 1], MSX2 [msh homeobox 2], MT2A [metallothionein 2A], MT3 [metallothionein 3], MT-ATP6 [mitochondrially encoded ATP synthase 6], MT-CO1 [mitochondrially encoded cytochrome c oxidase I], MT-CO2 [mitochondrially encoded cytochrome c oxidase II], MT-CO3 [mitochondrially encoded cytochrome c oxidase III], MTF1 [metal-regulatory transcription factor 1], MTHFD1 [methylenetetrahydrofolate dehydrogenase (NADP+dependent)₁, methenyltetrahydrofolate cyclohydrolase, formyltetrahydrofolate synthetase], MTHFD1L [methylenetetrahydrofolate dehydrogenase (NADP+dependent) 1-like], MTHFR [5 [10-methylenetetrahydrofolate reductase (NADPH)], MTL5 [metallothionein-like 5, testis-specific (tesmin)], MTMR14 [myotubularin related protein 14], MT-ND6 [mitochondrially encoded NADH dehydrogenase 6], MTNR1A [melatonin receptor 1A], MTNR1B [melatonin receptor 1 B], MTOR [mechanistic target of rapamycin (serine/threonine kinase)], MTR [5-methyltetrahydrofolate-homocysteine methyltransferase], MTRR [5-methyltetrahydrofolate-homocysteine methyltransferase reductase], MTTP [microsomal triglyceride transfer protein], MUC1 [mucin 1, cell surface associated], MUC16 [mucin 16, cell surface associated], MUC19 [mucin 19, oligomeric], MUC2 [mucin 2, oligomeric mucus/gel-forming], MUC3A [mucin 3A, cell surface associated], MUC5AC [mucin SAC, oligomeric mucus/gel-forming], MUSK [muscle, skeletal, receptor tyrosine kinase], MUT [methylmalonyl Coenzyme A mutase], MVK [mevalonate kinase], MVP [major vault protein], MX1 [myxovirus (influenza virus) resistance 1, interferon-inducible protein p78 (mouse)], MXD1 [MAX dimerization protein 1], MXI1 [MAX interactor 1], MYB [v-myb myeloblastosis viral oncogene homolog (avian)], MYC [v-myc myelocytomatosis viral oncogene homolog (avian)], MYCBP2 [MYC binding protein 2], MYCN [v-myc myelocytomatosis viral related oncogene, neuroblastoma derived (avian)], MYD88 [myeloid differentiation primary response gene (88)], MYF5 [myogenic factor 5], MYH10 [myosin, heavy chain 10, non-muscle], MYH14 [myosin, heavy chain 14, non-muscle], MYH7 [myosin, heavy chain 7, cardiac muscle, beta], MYL1 [myosin, light chain 1, alkali; skeletal, fast], MYL10 [myosin, light chain 10, regulatory], MYL12A [myosin, light chain 12A, regulatory, non-sarcomeric], MYL12B [myosin, light chain 12B, regulatory], MYL2 [myosin, light chain 2, regulatory, cardiac, slow], MYL3 [myosin, light chain 3, alkali; ventricular, skeletal, slow], MYL4 [myosin, light chain 4, alkali; atrial, embryonic], MYL5 [myosin, light chain 5, regulatory], MYL6 [myosin, light chain 6, alkali, smooth muscle and non-muscle], MYL6B [myosin, light chain 6B, alkali, smooth muscle and non-muscle], MYL7 [myosin, light chain 7, regulatory], MYL9 [myosin, light chain 9, regulatory], MYLK [myosin light chain kinase], MYLPF [myosin light chain, phosphorylatable, fast skeletal muscle], MYO1 D [myosin ID], MYO5A [myosin VA (heavy chain 12, myoxin)], MYOC [myocilin, trabecular meshwork inducible glucocorticoid response], MYOD1 [myogenic differentiation 1], MYOG [myogenin (myogenic factor 4)], MYOM2 [myomesin (M-protein) 2, 165 kDa], MYST3 [MYST histone acetyltransferase (monocytic leukemia) 3], NACA [nascent polypeptide-associated complex alpha subunit], NAGLU [N-acetylglucosaminidase, alpha-], NAIP [NLR family, apoptosis inhibitory protein], NAMPT [nicotinamide phosphoribosyltransferase], NANOG [Nanog homeobox], NANS [N-acetylneuraminic acid synthase], NAP1L2 [nucleosome assembly protein 1-like 2], NAPA [N-ethylmaleimide-sensitive factor attachment protein, alpha], NAPG [N-ethylmaleimide-sensitive factor attachment protein, gamma], NAT2 [N-acetyltransferase 2 (arylamine N-acetyltransferase)], NAV1 [neuron navigator 1], NAV3 [neuron navigator 3], NBEA [neurobeachin], NCALD [neurocalcin delta], NCAM1 [neural cell adhesion molecule 1], NCAM2 [neural cell adhesion molecule 2], NCF1 [neutrophil cytosolic factor 1], NCF2 [neutrophil cytosolic factor 2], NCK1 [NCK adaptor protein 1], NCK2 [NCK adaptor protein 2], NCKAP1 [NCK-associated protein 1], NCL [nucleolin], NCOA2 [nuclear receptor coactivator 2], NCOA3 [nuclear receptor coactivator 3], NCOR1 [nuclear receptor co-repressor 1], NCOR2 [nuclear receptor co-repressor 2], NDE1 [nudE nuclear distribution gene E homolog 1 (A. nidulans)], NDEL1 [nudE nuclear distribution gene E homolog (A. nidulans)-like 1], NDN [necdin homolog (mouse)], NDNL2 [necdin-like 2], NDP [Norrie disease (pseudoglioma)], NDUFA1 [NADH dehydrogenase (ubiquinone) 1 alpha subcomplex, 1, 7.5 kDa], NDUFAB1 [NADH dehydrogenase (ubiquinone)₁, alpha/beta subcomplex, 1, 8 kDa], NDUFS3 [NADH dehydrogenase (ubiquinone) Fe-S protein 3, 30 kDa (NADH-coenzyme Q reductase)], NDUFV3 [NADH dehydrogenase (ubiquinone) flavoprotein 3, 10 kDa], NEDD4 [neural precursor cell expressed, developmentally down-regulated 4], NEDD4L [neural precursor cell expressed, developmentally down-regulated 4-like], NEFH [neurofilament, heavy polypeptide], NEFL [neurofilament, light polypeptide], NEFM [neurofilament, medium polypeptide], NENF [neuron derived neurotrophic factor], NEO1 [neogenin homolog 1 (chicken)], NES [nestin], NET1 [neuroepithelial cell transforming 1], NEU1 [sialidase 1 (lysosomal sialidase)], NEU3 [sialidase 3 (membrane sialidase)], NEUROD1 [neurogenic differentiation 1], NEUROD4 [neurogenic differentiation 4], NEUROG1 [neurogenin 1], NEUROG2 [neurogenin 2], NF1 [neurofibromin 1], NF2 [neurofibromin 2 (merlin)], NFASC [neurofascin homolog (chicken)], NFAT5 [nuclear factor of activated T-cells 5, tonicity-responsive], NFATC1 [nuclear factor of activated T-cells, cytoplasmic, calcineurin-dependent 1], NFATC2 [nuclear factor of activated T-cells, cytoplasmic, calcineurin-dependent 2], NFATC3 [nuclear factor of activated T-cells, cytoplasmic, calcineurin-dependent 3], NFATC4 [nuclear factor of activated T-cells, cytoplasmic, calcineurin-dependent 4], NFE2L2 [nuclear factor (erythroid-derived 2)-like 2], NFIC [nuclear factor I/C(CCAAT-binding transcription factor)], NFIL3 [nuclear factor, interleukin 3 regulated], NFKB1 [nuclear factor of kappa light polypeptide gene enhancer in B-cells 1], NFKB2 [nuclear factor of kappa light polypeptide gene enhancer in B-cells 2 (p49/p100)], NFKBIA [nuclear factor of kappa light polypeptide gene enhancer in B-cells inhibitor, alpha], NFKBIB [nuclear factor of kappa light polypeptide gene enhancer in B-cells inhibitor, beta], NFKBIL1 [nuclear factor of kappa light polypeptide gene enhancer in B-cells inhibitor-like 1], NFYA [nuclear transcription factor Y, alpha], NFYB [nuclear transcription factor Y, beta], NGEF [neuronal guanine nucleotide exchange factor], NGF [nerve growth factor (beta polypeptide)], NGFR [nerve growth factor receptor (TNFR superfamily, member 16)], NGFRAP1 [nerve growth factor receptor (TNFRSF16) associated protein 1], NHLRC1 [NHL repeat containing 1], NINJ1 [ninjurin 1], NINJ2 [ninjurin 2], NIP7 [nuclear import 7 homolog (S. cerevisiae)], NIPA1 [non imprinted in Prader-Willi/Angelman syndrome 1], NIPA2 [non imprinted in Prader-Willi/Angelman syndrome 2], NIPAL1 [NIPA-like domain containing 1], NIPAL4 [NIPA-like domain containing 4], NIPSNAP1 [nipsnap homolog 1 (C. elegans)], NISCH [nischarin], NIT2 [nitrilase family, member 2], NKX2-1 [NK2 homeobox 1], NKX2-2 [NK2 homeobox 2], NLGN1 [neuroligin 1], NLGN2 [neuroligin 2], NLGN3 [neuroligin 3], NLGN4X [neuroligin 4, X-linked], NLGN4Y [neuroligin 4, P-linked], NLRP3 [NLR family, pyrin domain containing 3], NMB [neuromedin B], NME1 [non-metastatic cells 1, protein (NM23A) expressed in], NME2 [non-metastatic cells 2, protein (NM23B) expressed in], NME4 [non-metastatic cells 4, protein expressed in], NNAT [neuronatin], NOD1 [nucleotide-binding oligomerization domain containing 1], NOD2 [nucleotide-binding oligomerization domain containing 2], NOG [noggin], NOL6 [nucleolar protein family 6 (RNA-associated)], NOS1 [nitric oxide synthase 1 (neuronal)], NOS2 [nitric oxide synthase 2, inducible], NOS3 [nitric oxide synthase 3 (endothelial cell)], NOSTRIN [nitric oxide synthase trafficker], NOTCH1 [Notch homolog 1, translocation-associated (Drosophila)], NOTCH2 [Notch homolog 2 (Drosophila)], NOTCH3 [Notch homolog 3 (Drosophila)], NOV [nephroblastoma overexpressed gene], NOVA1 [neuro-oncological ventral antigen 1], NOVA2 [neuro-oncological ventral antigen 2], NOX4 [NADPH oxidase 4], NPAS4 [neuronal PAS domain protein 4], NPFF [neuropeptide FF-amide peptide precursor], NPHP1 [nephronophthisis 1 (juvenile)], NPHP4 [nephronophthisis 4], NPHS1 [nephrosis 1, congenital, Finnish type (nephrin)], NPM1 [nucleophosmin (nucleolar phosphoprotein B23, numatrin)], NPPA [natriuretic peptide precursor A], NPPB [natriuretic peptide precursor B], NPPC [natriuretic peptide precursor C], NPR1 [natriuretic peptide receptor A/guanylate cyclase A (atrionatriuretic peptide receptor A)], NPR3 [natriuretic peptide receptor C/guanylate cyclase C (atrionatriuretic peptide receptor C)], NPRL2 [nitrogen permease regulator-like 2 (S. cerevisiae)], NPTX1 [neuronal pentraxin I], NPTX2 [neuronal pentraxin II], NPY [neuropeptide Y], NPY1 R [neuropeptide Y receptor Y1], NPY2R [neuropeptide Y receptor Y2], NPY5R [neuropeptide Y receptor Y5], NQO1 [NAD(P)H dehydrogenase, quinone 1], NQO2 [NAD(P)H dehydrogenase, quinone 2], NROB1 [nuclear receptor subfamily 0, group B, member 1], NROB2 [nuclear receptor subfamily 0, group B, member 2], NR1H3 [nuclear receptor subfamily 1, group H, member 3], NR1H4 [nuclear receptor subfamily 1, group H, member 4], NR112 [nuclear receptor subfamily 1, group I, member 2], NR113 [nuclear receptor subfamily 1, group I, member 3], NR2C1 [nuclear receptor subfamily 2, group C, member 1], NR2C2 [nuclear receptor subfamily 2, group C, member 2], NR2E1 [nuclear receptor subfamily 2, group E, member 1], NR2F1 [nuclear receptor subfamily 2, group F, member 1], NR2F2 [nuclear receptor subfamily 2, group F, member 2], NR3C1 [nuclear receptor subfamily 3, group C, member 1 (glucocorticoid receptor)], NR3C2 [nuclear receptor subfamily 3, group C, member 2], NR4A2 [nuclear receptor subfamily 4, group A, member 2], NR4A3 [nuclear receptor subfamily 4, group A, member 3], NR5A1 [nuclear receptor subfamily 5, group A, member 1], NR6A1 [nuclear receptor subfamily 6, group A, member 1], NRAS [neuroblastoma RAS viral (v-ras) oncogene homolog], NRCAM [neuronal cell adhesion molecule], NRD1 [nardilysin (N-arginine dibasic convertase)], NRF1 [nuclear respiratory factor 1], NRG1 [neuregulin 1], NRIP1 [nuclear receptor interacting protein 1], NRN1 [neuritin 1], NRP1 [neuropilin 1], NRP2 [neuropilin 2], NRSN1 [neurensin 1], NRTN [neurturin], NRXN1 [neurexin 1], NRXN3 [neurexin 3], NSD1 [nuclear receptor binding SET domain protein 1], NSF [N-ethylmaleimide-sensitive factor], NSUN5 [NOP2/Sun domain family, member 5], NT5E [5′-nucleotidase, ecto (CD73)], NTF3 [neurotrophin 3], NTF4 [neurotrophin 4], NTHL1 [nth endonuclease III-like 1 (E. coli)], NTN1 [netrin 1], NTN3 [netrin 3], NTN4 [netrin 4], NTNG1 [netrin G1], NTRK1 [neurotrophic tyrosine kinase, receptor, type 1], NTRK2 [neurotrophic tyrosine kinase, receptor, type 2], NTRK3 [neurotrophic tyrosine kinase, receptor, type 3], NTS [neurotensin], NTSR1 [neurotensin receptor 1 (high affinity)], NUCB2 [nucleobindin 2], NUDC [nuclear distribution gene C homolog (A. nidulans)], NUDT6 [nudix (nucleoside diphosphate linked moiety X)-type motif 6], NUDT7 [nudix (nucleoside diphosphate linked moiety X)-type motif 7], NUMB [numb homolog (Drosophila)], NUP98 [nucleoporin 98 kDa], NUPR1 [nuclear protein, transcriptional regulator, 1], NXF1 [nuclear RNA export factor 1], NXNL1 [nucleoredoxin-like 1], OAT [ornithine aminotransferase], OCA2 [oculocutaneous albinism II], OCLN [occludin], OCM [oncomodulin], ODC1 [ornithine decarboxylase 1], OFD1 [oral-facial-digital syndrome 1], OGDH [oxoglutarate (alpha-ketoglutarate) dehydrogenase (lipoamide)], OLA1 [Obg-like ATPase 1], OLIG1 [oligodendrocyte transcription factor 1], OLIG2 [oligodendrocyte lineage transcription factor 2], OLR1 [oxidized low density lipoprotein (lectin-like) receptor 1], OMG [oligodendrocyte myelin glycoprotein], OPHN1 [oligophrenin 1], OPN1SW [opsin 1 (cone pigments), short-wave-sensitive], OPRD1 [opioid receptor, delta 1], OPRK1 [opioid receptor, kappa 1], OPRL1 [opiate receptor-like 1], OPRM1 [opioid receptor, mu 1], OPTN [optineurin], OSBP [oxysterol binding protein], OSBPL10 [oxysterol binding protein-like 10], OSBPL6 [oxysterol binding protein-like 6], OSM [oncostatin M], OTC [ornithine carbamoyltransferase], OTX2 [orthodenticle homeobox 2], OXA1L [oxidase (cytochrome c) assembly 1-like], OXT [oxytocin, prepropeptide], OXTR [oxytocin receptor], P2RX7 [purinergic receptor P2X, ligand-gated ion channel, 7], P2RY1 [purinergic receptor P2Y, G-protein coupled, 1], P2RY12 [purinergic receptor P2Y, G-protein coupled, 12], P2RY2 [purinergic receptor P2Y, G-protein coupled, 2], P4HB [prolyl 4-hydroxylase, beta polypeptide], PABPC1 [poly(A) binding protein, cytoplasmic 1], PADI4 [peptidyl arginine deiminase, type IV], PAEP [progestagen-associated endometrial protein], PAFAH1 B1 [platelet-activating factor acetylhydrolase 1b, regulatory subunit 1 (45 kDa)], PAFAH1 B2 [platelet-activating factor acetylhydrolase 1b, catalytic subunit 2 (30 kDa)], PAG1 [phosphoprotein associated with glycosphingolipid microdomains 1], PAH [phenylalanine hydroxylase], PAK1 [p21 protein (Cdc42/Rac)-activated kinase 1], PAK2 [p21 protein (Cdc42/Rac)-activated kinase 2], PAK3 [p21 protein (Cdc42/Rac)-activated kinase 3], PAK-4 [p21 protein (Cdc42/Rac)-activated kinase 4], PAK6 [p21 protein (Cdc42/Rac)-activated kinase 6], PAK7 [p21 protein (Cdc42/Rac)-activated kinase 7], PAPPA [pregnancy-associated plasma protein A, pappalysin 1], PAPPA2 [pappalysin 2], PARD6A [par-6 partitioning defective 6 homolog alpha (C. elegans)], PARG [poly (ADP-ribose) glycohydrolase], PARK2 [Parkinson disease (autosomal recessive, juvenile)₂, parkin], PARK7 [Parkinson disease (autosomal recessive, early onset) 7], PARN [poly(A)-specific ribonuclease (deadenylation nuclease)], PARP1 [poly (ADP-ribose) polymerase 1], PAWR [PRKC, apoptosis, WT1, regulator], PAX2 [paired box 2], PAX3 [paired box 3], PAX5 [paired box 5], PAX6 [paired box 6], PAX7 [paired box 7], PBX1 [pre-B-cell leukemia homeobox 1], PC [pyruvate carboxylase], PCDH10 [protocadherin 10], PCDH19 [protocadherin 19], PCDHAl2 [protocadherin alpha 12], PCK2 [phosphoenolpyruvate carboxykinase 2 (mitochondrial)], POLO [piccolo (presynaptic cytomatrix protein)], PCM1 [pericentriolar material 1], PCMT1 [protein-L-isoaspartate (D-aspartate) O-methyltransferase], PCNA [proliferating cell nuclear antigen], PCNT [pericentrin], PCP4 [Purkinje cell protein 4], PCSK7 [proprotein convertase subtilisin/kexin type 7], PDCD1 [programmed cell death 1], PDE11A [phosphodiesterase 11A], PDE3B [phosphodiesterase 3B, cGMP-inhibited], PDE4A [phosphodiesterase 4A, cAMP-specific (phosphodiesterase E2 dunce homolog, Drosophila)], PDE4B [phosphodiesterase 4B, cAMP-specific (phosphodiesterase E4 dunce homolog, Drosophila)], PDE4D [phosphodiesterase 4D, cAMP-specific (phosphodiesterase E3 dunce homolog, Drosophila)], PDE5A [phosphodiesterase 5A, cGMP-specific], PDE8A [phosphodiesterase 8A], PDGFA [platelet-derived growth factor alpha polypeptide], PDGFB [platelet-derived growth factor beta polypeptide (simian sarcoma viral (v-sis) oncogene homolog)], PDGFC [platelet derived growth factor C], PDGFD [platelet derived growth factor D], PDGFRA [platelet-derived growth factor receptor, alpha polypeptide], PDGFRB [platelet-derived growth factor receptor, beta polypeptide], PDHA1 [pyruvate dehydrogenase (lipoamide) alpha 1], PDIA2 [protein disulfide isomerase family A, member 2], PDIA3 [protein disulfide isomerase family A, member 3], PDLIM1 [PDZ and LIM domain 1], PDLIM7 [PDZ and LIM domain 7 (enigma)], PDP1 [pyruvate dehyrogenase phosphatase catalytic subunit 1], PDPN [podoplanin], PDXK [pyridoxal (pyridoxine, vitamin B6) kinase], PDXP [pyridoxal (pyridoxine, vitamin B6) phosphatase], PDYN [prodynorphin], PDZK1 [PDZ domain containing 1], PEBP1 [phosphatidylethanolamine binding protein 1], PECAM1 [platelet/endothelial cell adhesion molecule], PENK [proenkephalin], PER1 [period homolog 1 (Drosophila)], PER2 [period homolog 2 (Drosophila)], PEX13 [peroxisomal biogenesis factor 13], PEX2 [peroxisomal biogenesis factor 2], PEX5 [peroxisomal biogenesis factor 5], PEX7 [peroxisomal biogenesis factor 7], PF4 [platelet factor 4], PFAS [phosphoribosylformylglycinamidine synthase], PFKL [phosphofructokinase, liver], PFKM [phosphofructokinase, muscle], PFN1 [profilin 1], PFN2 [profilin 2], PFN3 [profilin 3], PFN4 [profilin family, member 4], PGAM2 [phosphoglycerate mutase 2 (muscle)], PGD [phosphogluconate dehydrogenase], PGF [placental growth factor], PGK1 [phosphoglycerate kinase 1], PGM1 [phosphoglucomutase 1], PGR [progesterone receptor], PHB [prohibitin], PHEX [phosphate regulating endopeptidase homolog, X-linked], PHF10 [PHD finger protein 10], PHF8 [PHD finger protein 8], PHGDH [phosphoglycerate dehydrogenase], PHKA2 [phosphorylase kinase, alpha 2 (liver)], PHLDA2 [pleckstrin homology-like domain, family A, member 2], PHOX2B [paired-like homeobox 2b], PHYH [phytanoyl-CoA 2-hydroxylase], PHYHIP [phytanoyl-CoA 2-hydroxylase interacting protein], PIAS1 [protein inhibitor of activated STAT, 1], PICALM [phosphatidylinositol binding clathrin assembly protein], PIGF [phosphatidylinositol glycan anchor biosynthesis, class F], PIGP [phosphatidylinositol glycan anchor biosynthesis, class P], PIK3C2A [phosphoinositide-3-kinase, class 2, alpha polypeptide], PIK3C2B [phosphoinositide-3-kinase, class 2, beta polypeptide], PIK3C2G [phosphoinositide-3-kinase, class 2, gamma polypeptide], PIK3C3 [phosphoinositide-3-kinase, class 3], PIK3CA [phosphoinositide-3-kinase, catalytic, alpha polypeptide], PIK3CB [phosphoinositide-3-kinase, catalytic, beta polypeptide], PIK3CD [phosphoinositide-3-kinase, catalytic, delta polypeptide], PIK3CG [phosphoinositide-3-kinase, catalytic, gamma polypeptide], PIK3R1 [phosphoinositide-3-kinase, regulatory subunit 1 (alpha)], PIK3R2 [phosphoinositide-3-kinase, regulatory subunit 2 (beta)], PIK3R3 [phosphoinositide-3-kinase, regulatory subunit 3 (gamma)], PIK3R4 [phosphoinositide-3-kinase, regulatory subunit 4], PIK3R5 [phosphoinositide-3-kinase, regulatory subunit 5], PINK1 [PTEN induced putative kinase 1], PITX1 [paired-like homeodomain 1], PITX2 [paired-like homeodomain 2], PITX3 [paired-like homeodomain 3], PKD1 [polycystic kidney disease 1 (autosomal dominant)], PKD2 [polycystic kidney disease 2 (autosomal dominant)], PKHD1 [polycystic kidney and hepatic disease 1 (autosomal recessive)], PKLR [pyruvate kinase, liver and RBC], PKN2 [protein kinase N2], PKNOX1 [PBX/knotted 1 homeobox 1], PL-5283 [PL-5283 protein], PLA2G10 [phospholipase A2, group X], PLA2G2A [phospholipase A2, group IIA (platelets, synovial fluid)], PLA2G4A [phospholipase A2, group IVA (cytosolic, calcium-dependent)], PLA2G6 [phospholipase A2, group VI (cytosolic, calcium-independent)], PLA2G7 [phospholipase A2, group VII (platelet-activating factor acetylhydrolase, plasma)], PLAC4 [placenta-specific 4], PLAG1 [pleiomorphic adenoma gene 1], PLAGL1 [pleiomorphic adenoma gene-like 1], PLAT [plasminogen activator, tissue], PLAU [plasminogen activator, urokinase], PLAUR [plasminogen activator, urokinase receptor], PLCB1 [phospholipase C, beta 1 (phosphoinositide-specific)], PLCB2 [phospholipase C, beta 2], PLCB3 [phospholipase C, beta 3 (phosphatidylinositol-specific)], PLCB4 [phospholipase C, beta 4], PLCG1 [phospholipase C, gamma 1], PLCG2 [phospholipase C, gamma 2 (phosphatidylinositol-specific)], PLCL1 [phospholipase C-like 1], PLD1 [phospholipase D1, phosphatidylcholine-specific], PLD2 [phospholipase D2], PLEK [pleckstrin], PLEKHH1 [pleckstrin homology domain containing, family H (with MyTH4 domain) member 1], PLG [plasminogen], PLIN1 [perilipin 1], PLK1 [polo-like kinase 1 (Drosophila)], PLOD1 [procollagen-lysine 1,2-oxoglutarate 5-dioxygenase 1], PLP1 [proteolipid protein 1], PLTP [phospholipid transfer protein], PLXNA1 [plexin A1], PLXNA2 [plexin A2], PLXNA3 [plexin A3], PLXNA4 [plexin A4], PLXNB1 [plexin B1], PLXNB2 [plexin B2], PLXNB3 [plexin B3], PLXNC1 [plexin C1], PLXND1 [plexin D1], PML [promyelocytic leukemia], PMP2 [peripheral myelin protein 2], PMP22 [peripheral myelin protein 22], PMS2 [PMS2 postmeiotic segregation increased 2 (S. cerevisiae)], PMVK [phosphomevalonate kinase], PNOC [prepronociceptin], PNP [purine nucleoside phosphorylase], PNPLA6 [patatin-like phospholipase domain containing 6], PNPO [pyridoxamine 5′-phosphate oxidase], POFUT2 [protein O-fucosyltransferase 2], POLB [polymerase (DNA directed), beta], POLR1C [polymerase (RNA) I polypeptide C, 30 kDa], POLR2A [polymerase (RNA) II (DNA directed) polypeptide A, 220 kDa], POLR3K [polymerase (RNA) III (DNA directed) polypeptide K, 12.3 kDa], POM121C [POM121 membrane glycoprotein C], POMC [proopiomelanocortin], POMGNT1 [protein O-linked mannose beta1 [2-N-acetylglucosaminyltransferase], POMT1 [protein-O-mannosyltransferase 1], PON1 [paraoxonase 1], PON2 [paraoxonase 2], POR [P450 (cytochrome) oxidoreductase], POSTN [periostin, osteoblast specific factor], POU1F1 [POU class 1 homeobox 1], POU2F1 [POU class 2 homeobox 1], POU3F4 [POU class 3 homeobox 4], POU4F1 [POU class 4 homeobox 1], POU4F2 [POU class 4 homeobox 2], POU4F3 [POU class 4 homeobox 3], POU5F1 [POU class 5 homeobox 1], PPA1 [pyrophosphatase (inorganic) 1], PPARA [peroxisome proliferator-activated receptor alpha], PPARD [peroxisome proliferator-activated receptor delta], PPARG [peroxisome proliferator-activated receptor gamma], PPARGC1A [peroxisome proliferator-activated receptor gamma, coactivator 1 alpha], PPAT [phosphoribosyl pyrophosphate amidotransferase], PPBP [pro-platelet basic protein (chemokine (C-X-C motif) ligand 7)], PPFIA1 [protein tyrosine phosphatase, receptor type, f polypeptide (PTPRF), interacting protein (liprin), alpha 1], PPFIA2 [protein tyrosine phosphatase, receptor type, f polypeptide (PTPRF), interacting protein (liprin), alpha 2], PPFIA3 [protein tyrosine phosphatase, receptor type, f polypeptide (PTPRF), interacting protein (liprin), alpha 3], PPFIBP1 [PTPRF interacting protein, binding protein 1 (liprin beta 1)], PPIC [peptidylprolyl isomerase C (cyclophilin C)], PPIG [peptidylprolyl isomerase G (cyclophilin G)], PPP1R15A [protein phosphatase 1, regulatory (inhibitor) subunit 15A], PPP1R1B [protein phosphatase 1, regulatory (inhibitor) subunit 1 B], PPP1 R9A [protein phosphatase 1, regulatory (inhibitor) subunit 9A], PPP1 R9B [protein phosphatase 1, regulatory (inhibitor) subunit 9B], PPP2CA [protein phosphatase 2, catalytic subunit, alpha isozyme], PPP2R4 [protein phosphatase 2A activator, regulatory subunit 4], PPP3CA [protein phosphatase 3, catalytic subunit, alpha isozyme], PPP3CB [protein phosphatase 3, catalytic subunit, beta isozyme], PPP3CC [protein phosphatase 3, catalytic subunit, gamma isozyme], PPP3R1 [protein phosphatase 3, regulatory subunit B, alpha], PPP3R2 [protein phosphatase 3, regulatory subunit B, beta], PPP4C [protein phosphatase 4, catalytic subunit], PPY [pancreatic polypeptide], PQBP1 [polyglutamine binding protein 1], PRAM1 [PML-RARA regulated adaptor molecule 1], PRAME [preferentially expressed antigen in melanoma], PRDM1 [PR domain containing 1, with ZNF domain], PRDM15 [PR domain containing 15], PRDM2 [PR domain containing 2, with ZNF domain], PRDX1 [peroxiredoxin 1], PRDX2 [peroxiredoxin 2], PRDX3 [peroxiredoxin 3], PRDX4 [peroxiredoxin 4], PRDX6 [peroxiredoxin 6], PRF1 [perforin 1 (pore forming protein)], PRKAA1 [protein kinase, AMP-activated, alpha 1 catalytic subunit], PRKAA2 [protein kinase, AMP-activated, alpha 2 catalytic subunit], PRKAB1 [protein kinase, AMP-activated, beta 1 non-catalytic subunit], PRKACA [protein kinase, cAMP-dependent, catalytic, alpha], PRKACB [protein kinase, cAMP-dependent, catalytic, beta], PRKACG [protein kinase, cAMP-dependent, catalytic, gamma], PRKAG1 [protein kinase, AMP-activated, gamma 1 non-catalytic subunit], PRKAG2 [protein kinase, AMP-activated, gamma 2 non-catalytic subunit], PRKAR1A [protein kinase, cAMP-dependent, regulatory, type I, alpha (tissue specific extinguisher 1)], PRKAR1B [protein kinase, cAMP-dependent, regulatory, type I, beta], PRKAR2A [protein kinase, cAMP-dependent, regulatory, type II, alpha], PRKAR2B [protein kinase, cAMP-dependent, regulatory, type II, beta], PRKCA [protein kinase C, alpha], PRKCB [protein kinase C, beta], PRKCD [protein kinase C, delta], PRKCE [protein kinase C, epsilon], PRKCG [protein kinase C, gamma], PRKCH [protein kinase C, eta], PRKCI [protein kinase C, iota], PRKCQ [protein kinase C, theta], PRKCZ [protein kinase C, zeta], PRKD1 [protein kinase D1], PRKDC [protein kinase, DNA-activated, catalytic polypeptide], PRKG1 [protein kinase, cGMP-dependent, type I], PRL [prolactin], PRLR [prolactin receptor], PRMT1 [protein arginine methyltransferase 1], PRNP [prion protein], PROC [protein C (inactivator of coagulation factors Va and VIIIa)], PROCR [protein C receptor, endothelial (EPCR)], PRODH [proline dehydrogenase (oxidase) 1], PROK1 [prokineticin 1], PROK2 [prokineticin 2], PROM1 [prominin 1], PRO51 [protein S (alpha)], PRPF40A [PRP40 pre-mRNA processing factor 40 homolog A (S. cerevisiae)], PRPF40B [PRP40 pre-mRNA processing factor 40 homolog B (S. cerevisiae)], PRPH [peripherin], PRPH2 [peripherin 2 (retinal degeneration, slow)], PRPS1 [phosphoribosyl pyrophosphate synthetase 1], PRRG4 [proline rich Gla (G-carboxyglutamic acid) 4 (transmembrane)], PRSS8 [protease, serine, 8], PRTN3 [proteinase 3], PRX [periaxin], PSAP [prosaposin], PSEN1 [presenilin 1], PSEN2 [presenilin 2 (Alzheimer disease 4)], PSG1 [pregnancy specific beta-1-glycoprotein 1], PSIP1 [PC4 and SFRS1 interacting protein 1], PSMA5 [proteasome (prosome, macropain) subunit, alpha type, 5], PSMA6 [proteasome (prosome, macropain) subunit, alpha type, 6], PSMB8 [proteasome (prosome, macropain) subunit, beta type, 8 (large multifunctional peptidase 7)], PSMB9 [proteasome (prosome, macropain) subunit, beta type, 9 (large multifunctional peptidase 2)], PSMC1 [proteasome (prosome, macropain) 26S subunit, ATPase, 1], PSMC4 [proteasome (prosome, macropain) 26S subunit, ATPase, 4], PSMD9 [proteasome (prosome, macropain) 26S subunit, non-ATPase, 9], PSME1 [proteasome (prosome, macropain) activator subunit 1 (PA28 alpha)], PSME2 [proteasome (prosome, macropain) activator subunit 2 (PA28 beta)], PSMG1 [proteasome (prosome, macropain) assembly chaperone 1], PSPH [phosphoserine phosphatase], PSPN [persephin], PSTPIP1 [proline-serine-threonine phosphatase interacting protein 1], PTAFR [platelet-activating factor receptor], PTCH1 [patched homolog 1 (Drosophila)], PTCH2 [patched homolog 2 (Drosophila)], PTEN [phosphatase and tensin homolog], PTF1A [pancreas specific transcription factor, 1a], PTGER1 [prostaglandin E receptor 1 (subtype EP1), 42 kDa], PTGER2 [prostaglandin E receptor 2 (subtype EP2), 53 kDa], PTGER3 [prostaglandin E receptor 3 (subtype EP3)], PTGER4 [prostaglandin E receptor 4 (subtype EP4)], PTGES [prostaglandin E synthase], PTGES2 [prostaglandin E synthase 2], PTGIR [prostaglandin 12 (prostacyclin) receptor (IP)], PTGS1 [prostaglandin-endoperoxide synthase 1 (prostaglandin G/H synthase and cyclooxygenase)], PTGS2 [prostaglandin-endoperoxide synthase 2 (prostaglandin G/H synthase and cyclooxygenase)], PTH [parathyroid hormone], PTH1 R [parathyroid hormone 1 receptor], PTHLH [parathyroid hormone-like hormone], PTK2 [PTK2 protein tyrosine kinase 2], PTK2B [PTK2B protein tyrosine kinase 2 beta], PTK7 [PTK7 protein tyrosine kinase 7], PTN [pleiotrophin], PTPN1 [protein tyrosine phosphatase, non-receptor type 1], PTPN11 [protein tyrosine phosphatase, non-receptor type 11], PTPN13 [protein tyrosine phosphatase, non-receptor type 13 (APO-1/CD95 (Fas)-associated phosphatase)], PTPN18 [protein tyrosine phosphatase, non-receptor type 18 (brain-derived)], PTPN2 [protein tyrosine phosphatase, non-receptor type 2], PTPN22 [protein tyrosine phosphatase, non-receptor type 22 (lymphoid)], PTPN6 [protein tyrosine phosphatase, non-receptor type 6], PTPN7 [protein tyrosine phosphatase, non-receptor type 7], PTPRA [protein tyrosine phosphatase, receptor type, A], PTPRB [protein tyrosine phosphatase, receptor type, B], PTPRC [protein tyrosine phosphatase, receptor type, C], PTPRD [protein tyrosine phosphatase, receptor type, D], PTPRE [protein tyrosine phosphatase, receptor type, E], PTPRF [protein tyrosine phosphatase, receptor type, F], PTPRJ [protein tyrosine phosphatase, receptor type, J], PTPRK [protein tyrosine phosphatase, receptor type, K], PTPRM [protein tyrosine phosphatase, receptor type, M], PTPRO [protein tyrosine phosphatase, receptor type, 0], PTPRS [protein tyrosine phosphatase, receptor type, S], PTPRT [protein tyrosine phosphatase, receptor type, T], PTPRU [protein tyrosine phosphatase, receptor type, U], PTPRZ1 [protein tyrosine phosphatase, receptor-type, Z polypeptide 1], PTS [6-pyruvoyltetrahydropterin synthase], PTTG1 [pituitary tumor-transforming 1], PVR [poliovirus receptor], PVRL1 [poliovirus receptor-related 1 (herpesvirus entry mediator C)], PWP2 [PWP2 periodic tryptophan protein homolog (yeast)], PXN [paxillin], PYCARD [PYD and CARD domain containing], PYGB [phosphorylase, glycogen; brain], PYGM [phosphorylase, glycogen, muscle], PYY [peptide YY], QDPR [quinoid dihydropteridine reductase], QKI [quaking homolog, KH domain RNA binding (mouse)], RAB11A [RAB11A, member RAS oncogene family], RAB11FIP5 [RAB11 family interacting protein 5 (class I)], RAB39B [RAB39B, member RAS oncogene family], RAB3A [RAB3A, member RAS oncogene family], RAB4A [RAB4A, member RAS oncogene family], RAB5A [RAB5A, member RAS oncogene family], RAB8A [RAB8A, member RAS oncogene family], RAB9A [RAB9A, member RAS oncogene family], RABEP1 [rabaptin, RAB GTPase binding effector protein 1], RABGEF1 [RAB guanine nucleotide exchange factor (GEF) 1], RAC1 [ras-related C3 botulinum toxin substrate 1 (rho family, small GTP binding protein Rac1)], RAC2 [ras-related C3 botulinum toxin substrate 2 (rho family, small GTP binding protein Rac2)], RAC3 [ras-related C3 botulinum toxin substrate 3 (rho family, small GTP binding protein Rac3)], RAD51 [RAD51 homolog (RecA homolog, E. coli) (S. cerevisiae)], RAF1 [v-raf-1 murine leukemia viral oncogene homolog 1], RAG1 [recombination activating gene 1], RAC2 [recombination activating gene 2], RAGE [renal tumor antigen], RALA [v-ral simian leukemia viral oncogene homolog A (ras related)], RALBP1 [ralA binding protein 1], RALGAPA2 [Ral GTPase activating protein, alpha subunit 2 (catalytic)], RALGAPB [Ral GTPase activating protein, beta subunit (non-catalytic)], RALGDS [ral guanine nucleotide dissociation stimulator], RAN [RAN, member RAS oncogene family], RAP1A [RAP1A, member of RAS oncogene family], RAP1 B [RAP1 B, member of RAS oncogene family], RAP1 GAP [RAP1 GTPase activating protein], RAPGEF3 [Rap guanine nucleotide exchange factor (GEF) 3], RAPGEF4 [Rap guanine nucleotide exchange factor (GEF) 4], RAPH1 [Ras association (RalGDS/AF-6) and pleckstrin homology domains 1], RAPSN [receptor-associated protein of the synapse], RARA [retinoic acid receptor, alpha], RARB [retinoic acid receptor, beta], RARG [retinoic acid receptor, gamma], RARS [arginyl-tRNA synthetase], RASA1 [RAS p21 protein activator (GTPase activating protein) 1], RASA2 [RAS p21 protein activator 2], RASGRF1 [Ras protein-specific guanine nucleotide-releasing factor 1], RASGRP1 [RAS guanyl releasing protein 1 (calcium and DAG-regulated)], RASSF1 [Ras association (RalGDS/AF-6) domain family member 1], RASSF5 [Ras association (RalGDS/AF-6) domain family member 5], RB1 [retinoblastoma 1], RBBP4 [retinoblastoma binding protein 4], RBM11 [RNA binding motif protein 11], RBM4 [RNA binding motif protein 4], RBM45 [RNA binding motif protein 45], RBP4 [retinol binding protein 4, plasma], RBPJ [recombination signal binding protein for immunoglobulin kappa J region], RCAN1 [regulator of calcineurin 1], RCAN2 [regulator of calcineurin 2], RCAN3 [ROAN family member 3], RCOR1 [REST corepressor 1], RDX [radixin], REEP3 [receptor accessory protein 3], REG1A [regenerating islet-derived 1 alpha], RELA [v-rel reticuloendotheliosis viral oncogene homolog A (avian)], RELN [reelin], REN [renin], REPIN1 [replication initiator 1], REST [RE1-silencing transcription factor], RET [ret proto-oncogene], RETN [resistin], RFC1 [replication factor C (activator 1) 1, 145 kDa], RFC2 [replication factor C (activator 1) 2, 40 kDa], RFX1 [regulatory factor X, 1 (influences HLA class II expression)], RGMA [RGM domain family, member A], RGMB [RGM domain family, member B], RGS3 [regulator of G-protein signaling 3], RHD [Rh blood group, D antigen], RHEB [Ras homolog enriched in brain], RHO [rhodopsin], RHOA [ras homolog gene family, member A], RHOB [ras homolog gene family, member B], RHOC [ras homolog gene family, member C], RHOD [ras homolog gene family, member D], RHOG [ras homolog gene family, member G (rho G)], RHOH [ras homolog gene family, member H], RICTOR [RPTOR independent companion of MTOR, complex 2], RIMS3 [regulating synaptic membrane exocytosis 3], RIPK1 [receptor (TNFRSF)-interacting serine-threonine kinase 1], RIPK2 [receptor-interacting serine-threonine kinase 2], RNASE1 [ribonuclease, RNase A family, 1 (pancreatic)], RNASE3 [ribonuclease, RNase A family, 3 (eosinophil cationic protein)], RNASEL [ribonuclease L (2′[5′-oligoisoadenylate synthetase-dependent)], RND1 [Rho family GTPase 1], RND2 [Rho family GTPase 2], RND3 [Rho family GTPase 3], RNF123 [ring finger protein 123], RNF128 [ring finger protein 128], RNF13 [ring finger protein 13], RNF135 [ring finger protein 135], RNF2 [ring finger protein 2], RNF6 [ring finger protein (C3H2C3 type) 6], RNH1 [ribonuclease/angiogenin inhibitor 1], RNPC3 [RNA-binding region (RNP1, RRM) containing 3], ROBO1 [roundabout, axon guidance receptor, homolog 1 (Drosophila)], ROBO2 [roundabout, axon guidance receptor, homolog 2 (Drosophila)], ROBO3 [roundabout, axon guidance receptor, homolog 3 (Drosophila)], ROBO4 [roundabout homolog 4, magic roundabout (Drosophila)], ROCK1 [Rho-associated, coiled-coil containing protein kinase 1], ROCK2 [Rho-associated, coiled-coil containing protein kinase 2], RPGR [retinitis pigmentosa GTPase regulator], RPGRIP1 [retinitis pigmentosa GTPase regulator interacting protein 1], RPGRIP1L [RPGRIP1-like], RPL10 [ribosomal protein L10], RPL24 [ribosomal protein L24], RPL5 [ribosomal protein L5], RPL7A [ribosomal protein L7a], RPLPO [ribosomal protein, large, PO], RPS17 [ribosomal protein S17], RPS17P3 [ribosomal protein S17 pseudogene 3], RPS19 [ribosomal protein S19], RPS27A [ribosomal protein S27a], RPS6 [ribosomal protein S6], RPS6KA1 [ribosomal protein S6 kinase, 90 kDa, polypeptide 1], RPS6KA3 [ribosomal protein S6 kinase, 90 kDa, polypeptide 3], RPS6KA6 [ribosomal protein S6 kinase, 90 kDa, polypeptide 6], RPS6 KB1 [ribosomal protein S6 kinase, 70 kDa, polypeptide 1], RRAS [related RAS viral (r-ras) oncogene homolog], RRAS2 [related RAS viral (r-ras) oncogene homolog 2], RRBP1 [ribosome binding protein 1 homolog 180 kDa (dog)], RRM1 [ribonucleotide reductase M1], RRM2 [ribonucleotide reductase M2], RRM2B [ribonucleotide reductase M2 B (TP53 inducible)], RTN4 [reticulon 4], RTN4R [reticulon 4 receptor], RUFY3 [RUN and FYVE domain containing 3], RUNX1 [runt-related transcription factor 1], RUNX1T1 [runt-related transcription factor 1; translocated to, 1 (cyclin D-related)], RUNX2 [runt-related transcription factor 2], RUNX3 [runt-related transcription factor 3], RUVBL2 [RuvB-like 2 (E. coli)], RXRA [retinoid X receptor, alpha], RYK [RYK receptor-like tyrosine kinase], RYR2 [ryanodine receptor 2 (cardiac)], RYR3 [ryanodine receptor 3], S100A1 [S100 calcium binding protein A1], S100A10 [S100 calcium binding protein A10], S100A12 [S100 calcium binding protein A12], S100A2 [S100 calcium binding protein A2], S100A4 [S100 calcium binding protein A4], S100A6 [S100 calcium binding protein A6], S100A7 [S100 calcium binding protein A7], S100A8 [S100 calcium binding protein A8], S100A9 [S100 calcium binding protein A9], S100B [S100 calcium binding protein B], SAA4 [serum amyloid A4, constitutive], SACS [spastic ataxia of Charlevoix-Saguenay (sacsin)], SAFB [scaffold attachment factor B], SAG [S-antigen; retina and pineal gland (arrestin)], SAMHD1 [SAM domain and HD domain 1], SATB2 [SATB homeobox 2], SBDS [Shwachman-Bodian-Diamond syndrome], SCARB1 [scavenger receptor class B, member 1], SCD [stearoyl-CoA desaturase (delta-9-desaturase)], SCD5 [stearoyl-CoA desaturase 5], SCG2 [secretogranin II], SCG5 [secretogranin V (7B2 protein)], SCGB1A1 [secretoglobin, family 1A, member 1 (uteroglobin)], SCN11A [sodium channel, voltage-gated, type XI, alpha subunit], SCN1A [sodium channel, voltage-gated, type I, alpha subunit], SCN2A [sodium channel, voltage-gated, type II, alpha subunit], SCN3A [sodium channel, voltage-gated, type III, alpha subunit], SCN5A [sodium channel, voltage-gated, type V, alpha subunit], SCN7A [sodium channel, voltage-gated, type VII, alpha], SCNN1B [sodium channel, nonvoltage-gated 1, beta], SCNN1G [sodium channel, nonvoltage-gated 1, gamma], SCP2 [sterol carrier protein 2], SCT [secretin], SCTR [secretin receptor], SCUBE1 [signal peptide, CUB domain, EGF-like 1], SDC2 [syndecan 2], SDC3 [syndecan 3], SDCBP [syndecan binding protein (syntenin)], SDHB [succinate dehydrogenase complex, subunit B, iron sulfur (Ip)], SDHD [succinate dehydrogenase complex, subunit D, integral membrane protein], SDS [serine dehydratase], SEC14L2 [SEC14-like 2 (S. cerevisiae)], SELE [selectin E], SELL [selectin L], SELP [selectin P (granule membrane protein 140 kDa, antigen CD62)], SELPLG [selectin P ligand], SEMA3A [sema domain, immunoglobulin domain (Ig), short basic domain, secreted, (semaphorin) 3A], SEMA3B [sema domain, immunoglobulin domain (Ig), short basic domain, secreted, (semaphorin) 3B], SEMA3C [sema domain, immunoglobulin domain (Ig), short basic domain, secreted, (semaphorin) 30], SEMA3D [sema domain, immunoglobulin domain (Ig), short basic domain, secreted, (semaphorin) 3D], SEMA3E [sema domain, immunoglobulin domain (Ig), short basic domain, secreted, (semaphorin) 3E], SEMA3F [sema domain, immunoglobulin domain (Ig), short basic domain, secreted, (semaphorin) 3F], SEMA3G [sema domain, immunoglobulin domain (Ig), short basic domain, secreted, (semaphorin) 3G], SEMA4A [sema domain, immunoglobulin domain (Ig), transmembrane domain (TM) and short cytoplasmic domain, (semaphorin) 4A], SEMA4B [sema domain, immunoglobulin domain (Ig), transmembrane domain (TM) and short cytoplasmic domain, (semaphorin) 4B], SEMA4C [sema domain, immunoglobulin domain (Ig), transmembrane domain (TM) and short cytoplasmic domain, (semaphorin) 40], SEMA4D [sema domain, immunoglobulin domain (Ig), transmembrane domain (TM) and short cytoplasmic domain, (semaphorin) 4D], SEMA4F [sema domain, immunoglobulin domain (Ig), transmembrane domain (TM) and short cytoplasmic domain, (semaphorin) 4F], SEMA4G [sema domain, immunoglobulin domain (Ig), transmembrane domain (TM) and short cytoplasmic domain, (semaphorin) 4G], SEMA5A [sema domain, seven thrombospondin repeats (type 1 and type 1-like), transmembrane domain (TM) and short cytoplasmic domain, (semaphorin) 5A], SEMA5B [sema domain, seven thrombospondin repeats (type 1 and type 1-like), transmembrane domain (TM) and short cytoplasmic domain, (semaphorin) 5B], SEMA6A [sema domain, transmembrane domain (TM), and cytoplasmic domain, (semaphorin) 6A], SEMA6B [sema domain, transmembrane domain (TM), and cytoplasmic domain, (semaphorin) 6B], SEMA6C [sema domain, transmembrane domain (TM), and cytoplasmic domain, (semaphorin) 60], SEMA6D [sema domain, transmembrane domain (TM), and cytoplasmic domain, (semaphorin) 6D], SEMA7A [semaphorin 7A, GPI membrane anchor (John Milton Hagen blood group)], SEPP1 [selenoprotein P, plasma, 1], SEPT2 [septin 2], SEPT4 [septin 4], SEPT5 [septin 5], SEPT6 [septin 6], SEPT7 [septin 7], SEPT9 [septin 9], SERPINA1 [serpin peptidase inhibitor, Glade A (alpha-1 antiproteinase, antitrypsin), member 1], SERPINA3 [serpin peptidase inhibitor, Glade A (alpha-1 antiproteinase, antitrypsin), member 3], SERPINA7 [serpin peptidase inhibitor, Glade A (alpha-1 antiproteinase, antitrypsin), member 7], SERPINB1 [serpin peptidase inhibitor, Glade B (ovalbumin), member 1], SERPINB2 [serpin peptidase inhibitor, Glade B (ovalbumin), member 2], SERPINB6 [serpin peptidase inhibitor, Glade B (ovalbumin), member 6], SERPINC1 [serpin peptidase inhibitor, Glade C (antithrombin), member 1], SERPINE1 [serpin peptidase inhibitor, Glade E (nexin, plasminogen activator inhibitor type 1), member 1], SERPINE2 [serpin peptidase inhibitor, Glade E (nexin, plasminogen activator inhibitor type 1), member 2], SERPINF1 [serpin peptidase inhibitor, Glade F (alpha-2 antiplasmin, pigment epithelium derived factor), member 1], SERPINH1 [serpin peptidase inhibitor, Glade H (heat shock protein 47), member 1, (collagen binding protein 1)], SERPINI1 [serpin peptidase inhibitor, Glade I (neuroserpin), member 1], SET [SET nuclear oncogene], SETX [senataxin], SEZ6L2 [seizure related 6 homolog (mouse)-like 2], SFPQ [splicing factor proline/glutamine-rich (polypyrimidine tract binding protein associated)], SFRP1 [secreted frizzled-related protein 1], SFRP4 [secreted frizzled-related protein 4], SFRS15 [splicing factor, arginine/serine-rich 15], SFTPA1 [surfactant protein A1], SFTPB [surfactant protein B], SFTPC [surfactant protein C], SGCB [sarcoglycan, beta (43 kDa dystrophin-associated glycoprotein)], SGCE [sarcoglycan, epsilon], SGK1 [serum/glucocorticoid regulated kinase 1], SH2B1 [SH2B adaptor protein 1], SH2B3 [SH2B adaptor protein 3], SH2D1A [SH2 domain containing 1A], SH3BGR [SH3 domain binding glutamic acid-rich protein], SH3BGRL [SH3 domain binding glutamic acid-rich protein like], SH3BP1 [SH3-domain binding protein 1], SH3GL1P2 [SH3-domain GRB2-like 1 pseudogene 2], SH3GL3 [SH3-domain GRB2-like 3], SH3 KBP1 [SH3-domain kinase binding protein 1], SH3PXD2A [SH3 and PX domains 2A], SHANK1 [SH3 and multiple ankyrin repeat domains 1], SHANK2 [SH3 and multiple ankyrin repeat domains 2], SHANK3 [SH3 and multiple ankyrin repeat domains 3], SHBG [sex hormone-binding globulin], SHC1 [SHC (Src homology 2 domain containing) transforming protein 1], SHC3 [SHC (Src homology 2 domain containing) transforming protein 3], SHH [sonic hedgehog homolog (Drosophila)], SHOC2 [soc-2 suppressor of clear homolog (C. elegans)], SI [sucrase-isomaltase (alpha-glucosidase)], SIAH1 [seven in absentia homolog 1 (Drosophila)], SIAH2 [seven in absentia homolog 2 (Drosophila)], SIGMAR1 [sigma non-opioid intracellular receptor 1], SILV [silver homolog (mouse)], SIM1 [single-minded homolog 1 (Drosophila)], SIM2 [single-minded homolog 2 (Drosophila)], SIP1 [survival of motor neuron protein interacting protein 1], SIRPA [signal-regulatory protein alpha], SIRT1 [sirtuin (silent mating type information regulation 2 homolog) 1 (S. cerevisiae)], SIRT4 [sirtuin (silent mating type information regulation 2 homolog) 4 (S. cerevisiae)], SIRT6 [sirtuin (silent mating type information regulation 2 homolog) 6 (S. cerevisiae)], SIX5 [SIX homeobox 5], SKI [v-ski sarcoma viral oncogene homolog (avian)], SKP2 [S-phase kinase-associated protein 2 (p45)], SLAMF6 [SLAM family member 6], SLC10A1 [solute carrier family 10 (sodium/bile acid cotransporter family), member 1], SLC11A2 [solute carrier family 11 (proton-coupled divalent metal ion transporters), member 2], SLC12A1 [solute carrier family 12 (sodium/potassium/chloride transporters), member 1], SLC12A2 [solute carrier family 12 (sodium/potassium/chloride transporters), member 2], SLC12A3 [solute carrier family 12 (sodium/chloride transporters), member 3], SLC12A5 [solute carrier family 12 (potassium/chloride transporter), member 5], SLC12A6 [solute carrier family 12 (potassium/chloride transporters), member 6], SLC13A1 [solute carrier family 13 (sodium/sulfate symporters), member 1], SLC15A1 [solute carrier family 15 (oligopeptide transporter), member 1], SLC16A2 [solute carrier family 16, member 2 (monocarboxylic acid transporter 8)], SLC17A5 [solute carrier family 17 (anion/sugar transporter), member 5], SLC17A7 [solute carrier family 17 (sodium-dependent inorganic phosphate cotransporter), member 7], SLC18A2 [solute carrier family 18 (vesicular monoamine), member 2], SLC18A3 [solute carrier family 18 (vesicular acetylcholine), member 3], SLC19A1 [solute carrier family 19 (folate transporter), member 1], SLC19A2 [solute carrier family 19 (thiamine transporter), member 2], SLC1A1 [solute carrier family 1 (neuronal/epithelial high affinity glutamate transporter, system Xag), member 1], SLC1A2 [solute carrier family 1 (glial high affinity glutamate transporter), member 2], SLC1A3 [solute carrier family 1 (glial high affinity glutamate transporter), member 3], SLC22A2 [solute carrier family 22 (organic cation transporter), member 2], SLC25A12 [solute carrier family 25 (mitochondrial carrier, Aralar), member 12], SLC25A13 [solute carrier family 25, member 13 (citrin)], SLC25A20 [solute carrier family 25 (carnitine/acylcarnitine translocase), member 20], SLC25A3 [solute carrier family 25 (mitochondrial carrier; phosphate carrier), member 3], SLC26A3 [solute carrier family 26, member 3], SLC27A1 [solute carrier family 27 (fatty acid transporter), member 1], SLC29A1 [solute carrier family 29 (nucleoside transporters), member 1], SLC2A1 [solute carrier family 2 (facilitated glucose transporter), member 1], SLC2A13 [solute carrier family 2 (facilitated glucose transporter), member 13], SLC2A2 [solute carrier family 2 (facilitated glucose transporter), member 2], SLC2A3 [solute carrier family 2 (facilitated glucose transporter), member 3], SLC2A4 [solute carrier family 2 (facilitated glucose transporter), member 4], SLC30A3 [solute carrier family 30 (zinc transporter), member 3], SLC30A4 [solute carrier family 30 (zinc transporter), member 4], SLC30A8 [solute carrier family 30 (zinc transporter), member 8], SLC31A1 [solute carrier family 31 (copper transporters), member 1], SLC32A1 [solute carrier family 32 (GABA vesicular transporter), member 1], SLC34A1 [solute carrier family 34 (sodium phosphate), member 1], SLC38A3 [solute carrier family 38, member 3], SLC39A2 [solute carrier family 39 (zinc transporter), member 2], SLC39A3 [solute carrier family 39 (zinc transporter), member 3], SLC40A1 [solute carrier family 40 (iron-regulated transporter), member 1], SLC4A11 [solute carrier family 4, sodium borate transporter, member 11], SLC5A3 [solute carrier family 5 (sodium/myo-inositol cotransporter), member 3], SLC5A8 [solute carrier family 5 (iodide transporter), member 8], SLC6A1 [solute carrier family 6 (neurotransmitter transporter, GABA), member 1], SLC6A14 [solute carrier family 6 (amino acid transporter), member 14], SLC6A2 [solute carrier family 6 (neurotransmitter transporter, noradrenalin), member 2], SLC6A3 [solute carrier family 6 (neurotransmitter transporter, dopamine), member 3], SLC6A4 [solute carrier family 6 (neurotransmitter transporter, serotonin), member 4], SLC6A8 [solute carrier family 6 (neurotransmitter transporter, creatine), member 8], SLC7A14 [solute carrier family 7 (cationic amino acid transporter, y-F system), member 14], SLC7A5 [solute carrier family 7 (cationic amino acid transporter, y+system), member 5], SLC9A2 [solute carrier family 9 (sodium/hydrogen exchanger), member 2], SLC9A3 [solute carrier family 9 (sodium/hydrogen exchanger), member 3], SLC9A3R1 [solute carrier family 9 (sodium/hydrogen exchanger), member 3 regulator 1], SLC9A3R2 [solute carrier family 9 (sodium/hydrogen exchanger), member 3 regulator 2], SLC9A6 [solute carrier family 9 (sodium/hydrogen exchanger), member 6], SLIT1 [slit homolog 1 (Drosophila)], SLIT2 [slit homolog 2 (Drosophila)], SLIT3 [slit homolog 3 (Drosophila)], SLITRK1 [SLIT and NTRK-like family, member 1], SLN [sarcolipin], SLPI [secretory leukocyte peptidase inhibitor], SMAD1 [SMAD family member 1], SMAD2 [SMAD family member 2], SMAD3

[SMAD family member 3], SMAD4 [SMAD family member 4], SMAD6 [SMAD family member 6], SMAD7 [SMAD family member 7], SMARCA1 [SWI/SNF related, matrix associated, actin dependent regulator of chromatin, subfamily a, member 1], SMARCA2 [SWI/SNF related, matrix associated, actin dependent regulator of chromatin, subfamily a, member 2], SMARCA4 [SWI/SNF related, matrix associated, actin dependent regulator of chromatin, subfamily a, member 4], SMARCA5 [SWI/SNF related, matrix associated, actin dependent regulator of chromatin, subfamily a, member 5], SMARCB1 [SWI/SNF related, matrix associated, actin dependent regulator of chromatin, subfamily b, member 1], SMARCC1 [SWI/SNF related, matrix associated, actin dependent regulator of chromatin, subfamily c, member 1], SMARCC2 [SWI/SNF related, matrix associated, actin dependent regulator of chromatin, subfamily c, member 2], SMARCD1 [SWI/SNF related, matrix associated, actin dependent regulator of chromatin, subfamily d, member 1], SMARCD3 [SWI/SNF related, matrix associated, actin dependent regulator of chromatin, subfamily d, member 3], SMARCE1 [SWI/SNF related, matrix associated, actin dependent regulator of chromatin, subfamily e, member 1], SMG1 [SMG1 homolog, phosphatidylinositol 3-kinase-related kinase (C. elegans)], SMN1 [survival of motor neuron 1, telomeric], SMO [smoothened homolog (Drosophila)], SMPD1 [sphingomyelin phosphodiesterase 1, acid lysosomal], SMS [spermine synthase], SNAI2 [snail homolog 2 (Drosophila)], SNAP25 [synaptosomal-associated protein, 25 kDa], SNCA [synuclein, alpha (non A4 component of amyloid precursor)], SNCAIP [synuclein, alpha interacting protein], SNOB [synuclein, beta], SNCG [synuclein, gamma (breast cancer-specific protein 1)], SNRPA [small nuclear ribonucleoprotein polypeptide A], SNRPN [small nuclear ribonucleoprotein polypeptide N], SNTG2 [syntrophin, gamma 2], SNURF [SNRPN upstream reading frame], SOAT1 [sterol O-acyltransferase 1], SOCS1 [suppressor of cytokine signaling 1], SOCS3 [suppressor of cytokine signaling 3], SOD1 [superoxide dismutase 1, soluble], SOD2 [superoxide dismutase 2, mitochondrial], SORBS3 [sorbin and SH3 domain containing 3], SORL1 [sortilin-related receptor, L(DLR class) A repeats-containing], SORT1 [sortilin 1], SOS1 [son of sevenless homolog 1 (Drosophila)], SOS2 [son of sevenless homolog 2 (Drosophila)], SOSTDC1 [sclerostin domain containing 1], SOX1 [SRY (sex determining region Y)-box 1], SOX10 [SRY (sex determining region Y)-box 10], SOX18 [SRY (sex determining region Y)-box 18], SOX2 [SRY (sex determining region Y)-box 2], SOX3 [SRY (sex determining region Y)-box 3], SOX9 [SRY (sex determining region Y)-box 9], SP1 [Sp1 transcription factor], SP3 [Sp3 transcription factor], SPANXB1 [SPANX family, member B1], SPANXC [SPANX family, member C], SPARC [secreted protein, acidic, cysteine-rich (osteonectin)], SPARCL1 [SPARC-like 1 (hevin)], SPAST [spastin], SPHK1 [sphingosine kinase 1], SPINK1 [serine peptidase inhibitor, Kazal type 1], SPINT2 [serine peptidase inhibitor, Kunitz type, 2], SPN [sialophorin], SPNS2 [spinster homolog 2 (Drosophila)], SPON2 [spondin 2, extracellular matrix protein], SPP1 [secreted phosphoprotein 1], SPRED2 [sprouty-related, EVH1 domain containing 2], SPRY2 [sprouty homolog 2 (Drosophila)], SPTA1 [spectrin, alpha, erythrocytic 1 (elliptocytosis 2)], SPTAN1 [spectrin, alpha, non-erythrocytic 1 (alpha-fodrin)], SPTB [spectrin, beta, erythrocytic], SPTBN1 [spectrin, beta, non-erythrocytic 1], SRC [v-src sarcoma (Schmidt-Ruppin A-2) viral oncogene homolog (avian)], SRCRB4D [scavenger receptor cysteine rich domain containing, group B (4 domains)], SRD5A1 [steroid-5-alpha-reductase, alpha polypeptide 1 (3-oxo-5 alpha-steroid delta 4-dehydrogenase alpha 1)], SREBF1 [sterol regulatory element binding transcription factor 1], SREBF2 [sterol regulatory element binding transcription factor 2], SRF [serum response factor (c-fos serum response element-binding transcription factor)], SRGAP1 [SLIT-ROBO Rho GTPase activating protein 1], SRGAP2 [SLIT-ROBO Rho GTPase activating protein 2], SRGAP3 [SLIT-ROBO Rho GTPase activating protein 3], SRPX [sushi-repeat-containing protein, X-linked], SRY [sex determining region Y], SSB [Sjogren syndrome antigen B (autoantigen La)], SSH1 [slingshot homolog 1 (Drosophila)], SSRP1 [structure specific recognition protein 1], SST [somatostatin], SSTR1 [somatostatin receptor 1], SSTR2 [somatostatin receptor 2], SSTR3 [somatostatin receptor 3], SSTR4 [somatostatin receptor 4], SSTR5 [somatostatin receptor 5], ST13 [suppression of tumorigenicity 13 (colon carcinoma) (Hsp70 interacting protein)], ST14 [suppression of tumorigenicity 14 (colon carcinoma)], ST6GAL1 [ST6 beta-galactosamide alpha-2 [6-sialyltranferase 1], ST7 [suppression of tumorigenicity 7], STAG2 [stromal antigen 2], STAG3 [stromal antigen 3], STAR [steroidogenic acute regulatory protein], STAT1 [signal transducer and activator of transcription 1, 91 kDa], STAT2 [signal transducer and activator of transcription 2, 113 kDa], STAT3 [signal transducer and activator of transcription 3 (acute-phase response factor)], STAT4 [signal transducer and activator of transcription 4], STAT5A [signal transducer and activator of transcription 5A], STAT5B [signal transducer and activator of transcription 5B], STAT6 [signal transducer and activator of transcription 6, interleukin-4 induced], STATH [statherin], STC1 [stanniocalcin 1], STIL [SCL/TAL1 interrupting locus], STIM1 [stromal interaction molecule 1], STK11 [serine/threonine kinase 11], STK24 [serine/threonine kinase 24 (STE20 homolog, yeast)], STK36 [serine/threonine kinase 36, fused homolog (Drosophila)], STK38 [serine/threonine kinase 38], STK38L [serine/threonine kinase 38 like], STK39 [serine threonine kinase 39 (STE20/SPS1 homolog, yeast)], STMN1 [stathmin 1], STMN2 [stathmin-like 2], STMN3 [stathmin-like 3], STMN4 [stathmin-like 4], STOML1 [stomatin (EPB72)-like 1], STS [steroid sulfatase (microsomal), isozyme S], STUB1 [STIP1 homology and U-box containing protein 1], STX1A [syntaxin 1A (brain)], STX3 [syntaxin 3], STYX [serine/threonine/tyrosine interacting protein], SUFU [suppressor of fused homolog (Drosophila)], SULT2A1 [sulfotransferase family, cytosolic, 2A, dehydroepiandrosterone (DHEA)-preferring, member 1], SUMO1 [SMT3 suppressor of mif two 3 homolog 1 (S. cerevisiae)], SUMO3 [SMT3 suppressor of mif two 3 homolog 3 (S. cerevisiae)], SUN1 [Sad1 and UNC84 domain containing 1], SUN2 [Sad1 and UNC84 domain containing 2], SUPT16H [suppressor of Ty 16 homolog (S. cerevisiae)], SUZ12P [suppressor of zeste 12 homolog pseudogene], SV2A [synaptic vesicle glycoprotein 2A], SYK [spleen tyrosine kinase], SYN1 [synapsin I], SYN2 [synapsin II], SYN3 [synapsin III], SYNGAP1 [synaptic Ras GTPase activating protein 1 homolog (rat)], SYNJ1 [synaptojanin 1], SYNPO₂ [synaptopodin 2], SYP [synaptophysin], SYT1 [synaptotagmin I], TAC1 [tachykinin, precursor 1], TAC3 [tachykinin 3], TACR1 [tachykinin receptor 1], TAF1 [TAF1 RNA polymerase II, TATA box binding protein (TBP)-associated factor, 250 kDa], TAF6 [TAF6 RNA polymerase II, TATA box binding protein (TBP)-associated factor, 80 kDa], TAGAP [T-cell activation RhoGTPase activating protein], TAGLN [transgelin], TAGLN3 [transgelin 3], TAOK2 [TAO kinase 2], TAP1 [transporter 1, ATP-binding cassette, sub-family B (MDR/TAP)], TAP2 [transporter 2, ATP-binding cassette, sub-family B (MDR/TAP)], TAPBP [TAP binding protein (tapasin)], TARDBP [TAR DNA binding protein], TARP [TCR gamma alternate reading frame protein], TAS2R1 [taste receptor, type 2, member 1], TAT [tyrosine aminotransferase], TBC1 D4 [TBC1 domain family, member 4], TBCB [tubulin folding cofactor B], TBCD [tubulin folding cofactor D], TBCE [tubulin folding cofactor E], TBL1Y [transducin (beta)-like 1, Y-linked], TBL2 [transducin (beta)-like 2], TBP [TATA box binding protein], TBPL2 [TATA box binding protein like 2], TBR1 [T-box, brain, 1], TBX1 [T-box 1], TBX21 [T-box 21], TBXA2R [thromboxane A2 receptor], TBXAS1 [thromboxane A synthase 1 (platelet)], TCEB3 [transcription elongation factor B (SIII), polypeptide 3 (110 kDa, elongin A)], TCF12 [transcription factor 12], TCF19 [transcription factor 19], TCF4 [transcription factor 4], TCF7 [transcription factor 7 (T-cell specific, HMG-box)], TCF7L2 [transcription factor 7-like 2 (T-cell specific, HMG-box)], TCHH [trichohyalin], TCN1 [transcobalamin I (vitamin B12 binding protein, R binder family)], TCN2 [transcobalamin II; macrocytic anemia], TCP1 [t-complex 1], TDO2 [tryptophan 2 [3-dioxygenase], TDRD3 [tudor domain containing 3], TEAD2 [TEA domain family member 2], TEAD4 [TEA domain family member 4], TEK [TEK tyrosine kinase, endothelial], TERF1 [telomeric repeat binding factor (NIMA-interacting) 1], TERF2 [telomeric repeat binding factor 2], TERT [telomerase reverse transcriptase], TET2 [tet oncogene family member 2], TF [transferrin], TFAM [transcription factor A, mitochondrial], TFAP2A [transcription factor AP-2 alpha (activating enhancer binding protein 2 alpha)], TFCP2 [transcription factor CP2], TFF1 [trefoil factor 1], TFF2 [trefoil factor 2], TFF3 [trefoil factor 3 (intestinal)], TFPI [tissue factor pathway inhibitor (lipoprotein-associated coagulation inhibitor)], TFPI2 [tissue factor pathway inhibitor 2], TFRC [transferrin receptor (p90, CD71)], TG [thyroglobulin], TGFα [transforming growth factor, alpha], TGFB1 [transforming growth factor, beta 1], TGFB1I1 [transforming growth factor beta 1 induced transcript 1], TGFB2 [transforming growth factor, beta 2], TGFB3 [transforming growth factor, beta 3], TGFBR1 [transforming growth factor, beta receptor 1], TGFBR2 [transforming growth factor, beta receptor II (70/80 kDa)], TGFBR3 [transforming growth factor, beta receptor III], TGIF1 [TGFB-induced factor homeobox 1], TGM2 [transglutaminase 2 (C polypeptide, protein-glutamine-gamma-glutamyltransferase)], TH [tyrosine hydroxylase], THAP1 [THAP domain containing, apoptosis associated protein 1], THBD [thrombomodulin], THBS1 [thrombospondin 1], THBS2 [thrombospondin 2], THBS4 [thrombospondin 4], THEM4 [thioesterase superfamily member 4], THPO [thrombopoietin], THRA [thyroid hormone receptor, alpha (erythroblastic leukemia viral (v-erb-a) oncogene homolog, avian)], THY1 [Thy-1 cell surface antigen], TIAM1 [T-cell lymphoma invasion and metastasis 1], TIAM2 [T-cell lymphoma invasion and metastasis 2], TIMP1 [TIMP metallopeptidase inhibitor 1], TIMP2 [TIMP metallopeptidase inhibitor 2], TIMP3 [TIMP metallopeptidase inhibitor 3], TINF2 [TERF1 (TRF1)-interacting nuclear factor 2], TJP1 [tight junction protein 1 (zona occludens 1)], TJP2 [tight junction protein 2 (zona occludens 2)], TK1 [thymidine kinase 1, soluble], TKT [transketolase], TLE1 [transducin-like enhancer of split 1 (E(sp1) homolog, Drosophila)], TLR1 [toll-like receptor 1], TLR2 [toll-like receptor 2], TLR3 [toll-like receptor 3], TLR4 [toll-like receptor 4], TLR5 [toll-like receptor 5], TLR7 [toll-like receptor 7], TLR8 [toll-like receptor 8], TLR9 [toll-like receptor 9], TLX3 [T-cell leukemia homeobox 3], TMEFF1 [transmembrane protein with EGF-like and two follistatin-like domains 1], TMEM100 [transmembrane protein 100], TMEM216 [transmembrane protein 216], TMEM50B [transmembrane protein 50B], TMEM67 [transmembrane protein 67], TMEM70 [transmembrane protein 70], TMEM87A [transmembrane protein 87A], TMOD2 [tropomodulin 2 (neuronal)], TMOD4 [tropomodulin 4 (muscle)], TMPRSS11A [transmembrane protease, serine 11A], TMPRSS15 [transmembrane protease, serine 15], TMPRSS2 [transmembrane protease, serine 2], TNC [tenascin C], TNF [tumor necrosis factor (TNF superfamily, member 2)], TNFAIP3 [tumor necrosis factor, alpha-induced protein 3], TNFRSF10A [tumor necrosis factor receptor superfamily, member 10a], TNFRSF10B [tumor necrosis factor receptor superfamily, member 10b], TNFRSF10C [tumor necrosis factor receptor superfamily, member 10c, decoy without an intracellular domain], TNFRSF10D [tumor necrosis factor receptor superfamily, member 10d, decoy with truncated death domain], TNFRSF11B [tumor necrosis factor receptor superfamily, member 11b], TNFRSF18 [tumor necrosis factor receptor superfamily, member 18], TNFRSF19 [tumor necrosis factor receptor superfamily, member 19], TNFRSF1A [tumor necrosis factor receptor superfamily, member 1A], TNFRSF1 B [tumor necrosis factor receptor superfamily, member 1 B], TNFRSF25 [tumor necrosis factor receptor superfamily, member 25], TNFRSF8 [tumor necrosis factor receptor superfamily, member 8], TNFSF10 [tumor necrosis factor (ligand) superfamily, member 10], TNFSF11 [tumor necrosis factor (ligand) superfamily, member 11], TNFSF13 [tumor necrosis factor (ligand) superfamily, member 13], TNFSF13B [tumor necrosis factor (ligand) superfamily, member 13b], TNFSF4 [tumor necrosis factor (ligand) superfamily, member 4], TNK2 [tyrosine kinase, non-receptor, 2], TNNI3 [troponin I type 3 (cardiac)], TNNT1 [troponin T type 1 (skeletal, slow)], TNNT2 [troponin T type 2 (cardiac)], TNR [tenascin R (restrictin, janusin)], TNS1 [tensin 1], TNS3 [tensin 3], TNXB [tenascin XB], TOLLIP [toll interacting protein], TOP1 [topoisomerase (DNA) I], TOP2A [topoisomerase (DNA) II alpha 170 kDa], TOP2B [topoisomerase (DNA) II beta 180 kDa], TOR1A [torsin family 1, member A (torsin A)], TP53 [tumor protein p53], TP53BP1 [tumor protein p53 binding protein 1], TP63 [tumor protein p63], TP73 [tumor protein p73], TPH1 [tryptophan hydroxylase 1], TPH2 [tryptophan hydroxylase 2], TPI1 [triosephosphate isomerase 1], TPO [thyroid peroxidase], TPT1 [tumor protein, translationally-controlled 1], TPTE [transmembrane phosphatase with tensin homology], TRADD [TNFRSF1A-associated via death domain], TRAF2 [TNF receptor-associated factor 2], TRAF3 [TNF receptor-associated factor 3], TRAF6 [TNF receptor-associated factor 6], TRAP1 [TNF receptor-associated protein 1], TREM1 [triggering receptor expressed on myeloid cells 1], TRH [thyrotropin-releasing hormone], TRIM21 [tripartite motif-containing 21], TRIM22 [tripartite motif-containing 22], TRIM26 [tripartite motif-containing 26], TRIM27 [tripartite motif-containing 27], TRIM50 [tripartite motif-containing 50], TR10 [triple functional domain (PTPRF interacting)], TRPA1 [transient receptor potential cation channel, subfamily A, member 1], TRPC1 [transient receptor potential cation channel, subfamily C, member 1], TRPC5 [transient receptor potential cation channel, subfamily C, member 5], TRPC6 [transient receptor potential cation channel, subfamily C, member 6], TRPM1 [transient receptor potential cation channel, subfamily M, member 1], TRPV1 [transient receptor potential cation channel, subfamily V, member 1], TRPV2 [transient receptor potential cation channel, subfamily V, member 2], TRRAP [transformation/transcription domain-associated protein], TSC1 [tuberous sclerosis 1], TSC2 [tuberous sclerosis 2], TSC22D3 [TSC22 domain family, member 3], TSG101 [tumor susceptibility gene 101], TSHR [thyroid stimulating hormone receptor], TSN [translin], TSPAN12 [tetraspanin 12], TSPAN7 [tetraspanin 7], TSPO [translocator protein (18 kDa)], TTC3 [tetratricopeptide repeat domain 3], TTF1 [transcription termination factor, RNA polymerase I], TTF2 [transcription termination factor, RNA polymerase II], TTN [titin], TTPA [tocopherol (alpha) transfer protein], TTR [transthyretin], TUB [tubby homolog (mouse)], TUBA1A [tubulin, alpha 1a], TUBA1B [tubulin, alpha 1b], TUBA1C [tubulin, alpha 1c], TUBA3C [tubulin, alpha 3c], TUBA3D [tubulin, alpha 3d], TUBA4A [tubulin, alpha 4a], TUBA8 [tubulin, alpha 8], TUBB [tubulin, beta], TUBB1 [tubulin, beta 1], TUBB2A [tubulin, beta 2A], TUBB2B [tubulin, beta 2B], TUBB2C [tubulin, beta 20], TUBB3 [tubulin, beta 3], TUBB4 [tubulin, beta 4], TUBB4Q [tubulin, beta polypeptide 4, member Q], TUBB6 [tubulin, beta 6], TUBGCP5 [tubulin, gamma complex associated protein 5], TUFM [Tu translation elongation factor, mitochondrial], TUSC3 [tumor suppressor candidate 3], TWIST1 [twist homolog 1 (Drosophila)], TXN [thioredoxin], TXNIP [thioredoxin interacting protein], TXNRD1 [thioredoxin reductase 1], TXNRD2 [thioredoxin reductase 2], TYK2 [tyrosine kinase 2], TYMP [thymidine phosphorylase], TYMS [thymidylate synthetase], TYR [tyrosinase (oculocutaneous albinism IA)], TYRO3 [TYRO3 protein tyrosine kinase], TYROBP [TYRO protein tyrosine kinase binding protein], TYRP1 [tyrosinase-related protein 1], U2AF1 [U2 small nuclear RNA auxiliary factor 1], UBA1 [ubiquitin-like modifier activating enzyme 1], UBA52 [ubiquitin A-52 residue ribosomal protein fusion product 1], UBB [ubiquitin B], UBC [ubiquitin C], UBE2A [ubiquitin-conjugating enzyme E2A (RAD6 homolog)], UBE2C [ubiquitin-conjugating enzyme E20], UBE2D2 [ubiquitin-conjugating enzyme E2D 2 (UBC4/5 homolog, yeast)], UBE2H [ubiquitin-conjugating enzyme E2H (UBC8 homolog, yeast)], UBE2I [ubiquitin-conjugating enzyme E2I (UBC9 homolog, yeast)], UBE3A [ubiquitin protein ligase E3A], UBL5 [ubiquitin-like 5], UCHL1 [ubiquitin carboxyl-terminal esterase L1 (ubiquitin thiolesterase)], UCN [urocortin], UCP1 [uncoupling protein 1 (mitochondrial, proton carrier)], UCP2 [uncoupling protein 2 (mitochondrial, proton carrier)], UCP3 [uncoupling protein 3 (mitochondrial, proton carrier)], UGT1A1 [UDP glucuronosyltransferase 1 family, polypeptide A1], UGT1A3 [UDP glucuronosyltransferase 1 family, polypeptide A3], ULK1 [unc-51-like kinase 1 (C. elegans)], UNC5A [unc-5 homolog A (C. elegans)], UNC5B [unc-5 homolog B (C. elegans)], UNC5C [unc-5 homolog C(C. elegans)], UNC5D [unc-5 homolog D (C. elegans)], UNG [uracil-DNA glycosylase], UPF3B [UPF3 regulator of nonsense transcripts homolog B (yeast)], UPK3B [uroplakin 3B], UPP2 [uridine phosphorylase 2], UQCRC1 [ubiquinol-cytochrome c reductase core protein I], USF1 [upstream transcription factor 1], USF2 [upstream transcription factor 2, c-fos interacting], USH2A [Usher syndrome 2A (autosomal recessive, mild)], USP1 [ubiquitin specific peptidase 1], USP15 [ubiquitin specific peptidase 15], USP25 [ubiquitin specific peptidase 25], USP29 [ubiquitin specific peptidase 29], USP33 [ubiquitin specific peptidase 33], USP4 [ubiquitin specific peptidase 4 (proto-oncogene)], USP5 [ubiquitin specific peptidase 5 (isopeptidase T)], USP9X [ubiquitin specific peptidase 9, X-linked], USP9Y [ubiquitin specific peptidase 9, Y-linked], UTRN [utrophin], UXT [ubiquitously-expressed transcript], VAMP7 [vesicle-associated membrane protein 7], VASP [vasodilator-stimulated phosphoprotein], VAV1 [vav 1 guanine nucleotide exchange factor], VAV2 [vav 2 guanine nucleotide exchange factor], VAX1 [ventral anterior homeobox 1], VCAM1 [vascular cell adhesion molecule 1], VCL [vinculin], VDAC1 [voltage-dependent anion channel 1], VDAC2 [voltage-dependent anion channel 2], VDR [vitamin D (1 [25-dihydroxyvitamin D3) receptor], VEGFA [vascular endothelial growth factor A], VEGFB [vascular endothelial growth factor B], VEGFC [vascular endothelial growth factor C], VGF [VGF nerve growth factor inducible], VHL [von Hippel-Lindau tumor suppressor], VIM [vimentin], VIP [vasoactive intestinal peptide], VIPR1 [vasoactive intestinal peptide receptor 1], VIPR2 [vasoactive intestinal peptide receptor 2], VKORC1 [vitamin K epoxide reductase complex, subunit 1], VLDLR [very low density lipoprotein receptor], VPS29 [vacuolar protein sorting 29 homolog (S. cerevisiae)], VSIG4 [V-set and immunoglobulin domain containing 4], VSX1 [visual system homeobox 1], VTN [vitronectin], VWC2 [von Willebrand factor C domain containing 2], VWF [von Willebrand factor], WAS [Wiskott-Aldrich syndrome (eczema-thrombocytopenia)], WASF1 [WAS protein family, member 1], WASF2 [WAS protein family, member 2], WASL [Wiskott-Aldrich syndrome-like], WBSCR16 [Williams-Beuren syndrome chromosome region 16], WBSCR17 [Williams-Beuren syndrome chromosome region 17], WBSCR22 [Williams Beuren syndrome chromosome region 22], WBSCR27 [Williams Beuren syndrome chromosome region 27], WBSCR28 [Williams-Beuren syndrome chromosome region 28], WDR4 [WD repeat domain 4], WEE1 [WEE1 homolog (S. pombe)], WHAMM [WAS protein homolog associated with actin, golgi membranes and microtubules], WIPF1 [WAS/WASL interacting protein family, member 1], WIPF3 [WAS/WASL interacting protein family, member 3], WNK3 [WNK lysine deficient protein kinase 3], WNT1 [wingless-type MMTV integration site family, member 1], WNT10A [wingless-type MMTV integration site family, member 10A], WNT10B [wingless-type MMTV integration site family, member 10B], WNT11 [wingless-type MMTV integration site family, member 11], WNT16 [wingless-type MMTV integration site family, member 16], WNT2 [wingless-type MMTV integration site family member 2], WNT2B [wingless-type MMTV integration site family, member 2B], WNT3 [wingless-type MMTV integration site family, member 3], WNT3A [wingless-type MMTV integration site family, member 3A], WNT4 [wingless-type MMTV integration site family, member 4], WNT5A [wingless-type MMTV integration site family, member 5A], WNT5B [wingless-type MMTV integration site family, member 5B], WNT6 [wingless-type MMTV integration site family, member 6], WNT7A [wingless-type MMTV integration site family, member 7A], WNT7B [wingless-type MMTV integration site family, member 7B], WNT8A [wingless-type MMTV integration site family, member 8A], WNT8B [wingless-type MMTV integration site family, member 8B], WNT9A [wingless-type MMTV integration site family, member 9A], WNT9B [wingless-type MMTV integration site family, member 9B], WRB [tryptophan rich basic protein], WRN [Werner syndrome, RecQ helicase-like], WT1 [Wilms tumor 1], XBP1 [X-box binding protein 1], XCL1 [chemokine (C motif) ligand 1], XDH [xanthine dehydrogenase], XIAP [X-linked inhibitor of apoptosis], XIRP2 [xin actin-binding repeat containing 2], XPC [xeroderma pigmentosum, complementation group C], XRCC1 [X-ray repair complementing defective repair in Chinese hamster cells 1], XRCC5 [X-ray repair complementing defective repair in Chinese hamster cells 5 (double-strand-break rejoining)], XRCC6 [X-ray repair complementing defective repair in Chinese hamster cells 6], XRN1 [5′-3′ exoribonuclease 1], YBX1 [Y box binding protein 1], YWHAB [tyrosine 3-monooxygenase/tryptophan 5-monooxygenase activation protein, beta polypeptide], YWHAE [tyrosine 3-monooxygenase/tryptophan 5-monooxygenase activation protein, epsilon polypeptide], YWHAG [tyrosine 3-monooxygenase/tryptophan 5-monooxygenase activation protein, gamma polypeptide], YWHAQ [tyrosine 3-monooxygenase/tryptophan 5-monooxygenase activation protein, theta polypeptide], YWHAZ [tyrosine 3-monooxygenase/tryptophan 5-monooxygenase activation protein, zeta polypeptide], ZAP70 [zeta-chain (TCR) associated protein kinase 70 kDa], ZBTB16 [zinc finger and BTB domain containing 16], ZBTB33 [zinc finger and BTB domain containing 33], ZC3H12A [zinc finger CCCH-type containing 12A], ZEB1 [zinc finger E-box binding homeobox 1], ZEB2 [zinc finger E-box binding homeobox 2], ZFP161 [zinc finger protein 161 homolog (mouse)], ZFP36 [zinc finger protein 36, C3H type, homolog (mouse)], ZFP42 [zinc finger protein 42 homolog (mouse)], ZFP57 [zinc finger protein 57 homolog (mouse)], ZFPM1 [zinc finger protein, multitype 1], ZFPM2 [zinc finger protein, multitype 2], ZFY [zinc finger protein, Y-linked], ZFYVE9 [zinc finger, FYVE domain containing 9], ZIC1 [Zic family member 1 (odd-paired homolog, Drosophila)], ZIC2 [Zic family member 2 (odd-paired homolog, Drosophila)], ZIC3 [Zic family member 3 (odd-paired homolog, Drosophila)], ZMPSTE24 [zinc metallopeptidase (STE24 homolog, S. cerevisiae)], ZNF148 [zinc finger protein 148], ZNF184 [zinc finger protein 184], ZNF225 [zinc finger protein 225], ZNF256 [zinc finger protein 256], ZNF333 [zinc finger protein 333], ZNF385B [zinc finger protein 385B], ZNF44 [zinc finger protein 44], ZNF521 [zinc finger protein 521], ZNF673 [zinc finger family member 673], ZNF79 [zinc finger protein 79], ZNF84 [zinc finger protein 84], ZW10 [ZW10, kinetochore associated, homolog (Drosophila)], and ZYX [zyxin].

Preferred neurodevelopmental genes may include BMP4 (bone morphogenetic protein 4); CHRD (chordin); NOG (noggin); WNT2 (wingless-type MMTV integration site family member 2); WNT2B (wingless-type MMTV integration site family, member 2B); WNT3A (wingless-type MMTV integration site family, member 3A); WNT4(wingless-type MMTV integration site family, member 4); WNT5A (wingless-type MMTV integration site family, member 5A); WNT6 (wingless-type MMTV integration site family, member 6); WNT7B (wingless-type MMTV integration site family, member 7B); WNT8B (wingless-type MMTV integration site family, member 8B); WNT9A (wingless-type MMTV integration site family, member 9A); WNT9B (wingless-type MMTV integration site family, member 9B); WNT10A (wingless-type MMTV integration site family, member 10A); WNT10B (wingless-type MMTV integration site family, member 10B); WNT16 (wingless-type MMTV integration site family, member 16); OTX2 (orthodenticle homeobox 2); GBX2 (gastrulation brain homeobox 2); FGF8 (fibroblast growth factor 8 (androgen-induced)); RELN (reelin); DAB1 (disabled homolog 1 (Drosophila)); POU4F1 (POU class 4 homeobox 1); and NUMB (numb homolog (Drosophila).

(i) BMP4

BMP4 (bone morphogenetic protein 4) is a critical signaling protein secreted from the dorsal part of an embryonic notochord and involved in the establishment of a dorsal-ventral axis. Inhibition of the BMP4 signal by other signaling proteins has been shown to cause the ectoderm to differentiate into the neural plate, the precursor tissue for the brain and spinal cord. Disregulation of BMP4 in an animal model was associated with the development of holoprosencephaly (HPE), a common malformation of the forebrain, and enteric nervous system disorders such as Hirschsprung's disease and intestinal neuronal dysplasia. Four missense mutations in BMP4 were detected in a population of human spina bifida aperta patients.

(ii) CHRD

CHRD (chordin) is a polypeptide that functions as a BMP antagonist to promote mammalian neural crest development and to regulate subsequent neural crest cell emigration from the neural tube. Mouse models lacking CHRD developed several lethal neonatal phenotypes including cyclopia, holoprosencephaly, and rostral truncations of the brain and craniofacial skeleton. In animal models, disruption of BMP signaling using exogenous CHRD is associated with the development of holoprosencephaly (HPE).

(iii) NOG

NOG (noggin) is a polypeptide that functions as a BMP4 antagonist to promote mammalian neural crest development and to regulate subsequent neural crest cell emigration from the neural tube, in a manner similar to CHRD. Experimental results of mouse knockout models lacking noggin suggest that NOG is involved in numerous developmental processes, such as neural tube fusion and joint formation. Enhanced caudal NOG expression plays a role in the lack of neurogenic potential characterizing the caudal-most neural crest cells. A missense mutation in NOG was detected in a population of human spina bifida aperta patients.

(iv) WNT genes

The WNT genes encode a multitude of morphogenetic signaling proteins also involved in dorso-ventral patterning of the developing neural tube. WNT proteins are involved in the canonical Wnt/β-catenin pathway, which acts in the roof plate, the dorsal-most region of the neural tube. In addition, WNT proteins have diverse roles in axon guidance processes. WNT proteins include WNT2 (wingless-type MMTV integration site family member 2); WNT2B (wingless-type MMTV integration site family, member 2B); WNT3A (wingless-type MMTV integration site family, member 3A); WNT4(wingless-type MMTV integration site family, member 4); WNT5A (wingless-type MMTV integration site family, member 5A); WNT6 (wingless-type MMTV integration site family, member 6); WNT7B (wingless-type MMTV integration site family, member 7B); WNT8B (wingless-type MMTV integration site family, member 8B); WNT9A (wingless-type MMTV integration site family, member 9A); WNT9B (wingless-type MMTV integration site family, member 9B); WNT10A (wingless-type MMTV integration site family, member 10A); WNT10B (wingless-type MMTV integration site family, member 10B); and WNT16 (wingless-type MMTV integration site family, member 16). Although the function of each WNT protein has not been affirmatively established for all WNT proteins, experimentation using animal models has lead to many insights as to the function of individual WNT proteins.

WNT signals are implicated in morphogenesis of neural tissues. During early differentiation of NT2 cells, WNT3A, WNT8A, WNT8B, WNT10B and WNT11 are down-regulated, and WNT2, WNT7B and WNT14B are up-regulated.

WNT2B produces two alternative transcript variants, and functions as a stem cell factor for neural or retinal progenitor cells during embryogenesis

WNT3A is thought to promote neural progenitor cell proliferation by inducing a shortened cell cycle in the progenitor cells. However, may also be involved in the neuronal differentiation process.

WNT4 has been identified as an inhibitor of embryonic stem cell neurogenesis in mouse embryonic stem (ES) cells. WNT4 also acts as an axon guidance molecule to attract ascending sensory axons in during development, and has been observed to be acutely induced in areas adjacent to the lesion of a spinal cord injury.

WNT5A is a WNT ligand that usually activates noncanonical Wnt signaling pathways during early development. WNT5A also acts as an axon guidance molecule to repel descending corticospinal tract (CST) axons during development, and has been observed to be robustly and diffusely expressed along the length of the spinal cord after an acute spinal injury.

WNT6 has been identified as an inhibitor of embryonic stem cell neurogenesis in mouse embryonic stem (ES) cells.

WNT7B acts as an axon guidance molecule and has been shown to mediate the establishment of synaptic connections between peripheral olfactory axons and CNS neurons.

WNT8B had been shown to modulate the number of dopaminergic (DA) neurons within the diencephalic anlage of the neural plate during primary neurogenesis. WNT8B was also shown to be significantly involved in neurogenesis in the developing hypothalamus region. The expression patterns of human WNT8B and the mouse wnt8b homolog appears to be highly similar and restricted to the developing brain. The chromosomal location of WNT8B to 10q24 suggests it as a candidate gene for partial epilepsy.

(v) OTX2

OTX2 (orthodenticle homeobox 2) encodes a member of the bicoid sub-family of homeodomain-containing transcription factors. The encoded protein acts as a transcription factor and may play a role in brain and sensory organ development. OTX2 was shown to provide the crucial anterior-posterior positional information for the generation of red nucleus neurons in the murine midbrain.

(vi) GBX2

GBX2 (gastrulation brain homeobox 2) is a protein involved in neural crest development and differentiation. The responsive elements of GBX2 respond directly to Wnt/beta-catenin signaling. and has been has previously been implicated in posteriorization of the neural crest cells. A role for GBX2 in neural fold patterning has also been suggested.

Mutual inhibition between GBX2 and OTX2, which are respectively expressed in the anterior and posterior parts of the neural plate, has been shown to position the prospective midbrain-hindbrain junction, and misexpression of GBX2 in the mesencephalon results on the deletion of the midbrain and cerebellum in a mouse model.

(vii) FGF8

FGF8 is a member of the fibroblast growth factor (FGF) family that plays an important role in early neural development. Expression of FGF8 was observed to transiently and rapidly increase in the early stages during retinoic acid-induced neural differentiation, followed by a decline in expression. FGF8 also acts as an axonal guidance molecule; exogenous FGF8 placed within the midbrain-hindbrain boundary (MHB) was shown to repel axons growing from midbrain neurons (mDANs).

(viii) RELN

RELN (reelin) is a protein that helps In addition, RELN modulates synaptic plasticity by enhancing the induction and maintenance of long-term potentiation. RELN is found in the brain, spinal cord, blood, and other body organs and tissues. RELN has been tentatively implicated in pathogenesis of several brain diseases. RELN expression is significantly lower in schizophrenia and psychotic bipolar disorder populations, but the cause remains uncertain as studies show that psychotropic medication itself affects RELN expression. Total lack of reelin causes a form of lissencephaly. Reelin may also play a role in Alzheimer's disease, temporal lobe epilepsy and autism.

(ix) DAB1

DAB1 (disabled-1) is a key regulator of reelin signaling. DAB1 functions downstream of RELN in a signaling pathway that controls cell positioning in the developing brain and during adult neurogenesis. DAB1 has been implicated in neuronal development in flies, and in mice, DAB1 mutation results in the scrambler mouse phenotype. Targeted disruption of the DAB1 gene in the mice disturbed neuronal layering in the cerebral cortex, hippocampus, and cerebellum, causing a reeler-like phenotype.

(x) POU4F1

POU4F1 (POU class 4 homeobox 1) is a class IV POU domain-containing transcription factor that is highly expressed in the developing sensory nervous system. POU4F1 is expressed in developing sensory neurons at all levels of the neural axis, including the trigeminal ganglion, hindbrain sensory ganglia, and dorsal root ganglia Mice lacking the POU4F1 exhibit growth defects in trigeminal axons, undergo extensive sensory cell death in late gestation, and die at birth.

(xi) NUMB

NUMB (numb homolog (Drosophila) is a protein known to play a role in the determination of cell fates during development. NUMB has been shown to regulate neurogenesis in the developing nervous systems including the maintenance of the self-renewal properties of neural progenitor cells in the vertebrate neural tube. NUMB mutations in mice results in premature depletion of neural stem/progenitor cells in mice. Inducible mouse mutants lacking NUMB in developing sensory ganglia show a severe reduction in axonal arborization in afferent fibers, but no deficit in neurogenesis. Mice embryos completely lacking functional NUMB exhibit severe defects in cranial neural tube closure and precocious neuron production in the forebrain. In addition, NUMB is expressed in neurons and glial cells after a spinal injury in a time-dependent manner in a mouse model.

The identity of the neurodevelopmental protein in which a chromosomal sequence is edited can and will vary. In general, the exemplary neurodevelopmental protein in which a chromosomal sequence is edited may be BMP4, CHRD, NOG, WNT2, WNT2B, WNT3A, WNT4, WNT5A, WNT6, WNT7B, WNT8B, WNT9A, WNT9B, WNT10A, WNT10B, WNT16, OTX2, GBX2, FGF8, RELN, DAB1, POU4F1, NUMB and any combination thereof.

In one aspect, the chromosomal sequences of any combination of any two neurodevelopmental proteins may be edited using a zinc finger nuclease-mediated process. In other aspects, the chromosomal sequences of any combination of any three exemplary neurodevelopmental proteins, any four exemplary neurodevelopmental proteins, any five exemplary neurodevelopmental proteins, any six exemplary neurodevelopmental proteins, any seven exemplary neurodevelopmental proteins, any eight exemplary neurodevelopmental proteins, any nine exemplary neurodevelopmental proteins, any ten exemplary neurodevelopmental proteins, any eleven exemplary neurodevelopmental proteins, any twelve exemplary neurodevelopmental proteins, any thirteen exemplary neurodevelopmental proteins, any fourteen exemplary neurodevelopmental proteins, any fifteen exemplary neurodevelopmental proteins, any sixteen exemplary neurodevelopmental proteins, any seventeen exemplary neurodevelopmental proteins, any eighteen exemplary neurodevelopmental proteins, any nineteen exemplary neurodevelopmental proteins, any twenty exemplary neurodevelopmental proteins, any twenty-one exemplary neurodevelopmental proteins, or any twenty-two exemplary neurodevelopmental proteins may be edited using a zinc finger nuclease-mediated process. In yet another aspect, the chromosomal sequences of any combination of all twenty-two exemplary neurodevelopmental proteins may be edited using a zinc finger nuclease-mediated process.

Exemplary genetically modified animals may comprise one, two, three, four, five, six, seven, eight, nine, ten, eleven, twelve, thirteen, fourteen, fifteen, sixteen, seventeen, eighteen, nineteen, twenty, or twenty-one, twenty-two or twenty-three inactivated chromosomal sequences encoding a neurodevelopmental protein and zero, one, two, three, four, five, six, seven, eight, nine, ten, eleven, twelve, thirteen, fourteen, fifteen, sixteen, seventeen, eighteen, nineteen, twenty, twenty-one, twenty-two or twenty-three chromosomally integrated sequences encoding orthologous or modified neurodevelopmental proteins.

### (b) Animals

The term “animal,” as used herein, refers to a non-human animal. The animal may be an embryo, a juvenile, or an adult. Suitable animals include vertebrates such as mammals, birds, reptiles, amphibians, and fish. Examples of suitable mammals include without limit rodents, companion animals, livestock, and primates. Non-limiting examples of rodents include mice, rats, hamsters, gerbils, and guinea pigs. Suitable companion animals include but are not limited to cats, dogs, rabbits, hedgehogs, and ferrets. Non-limiting examples of livestock include horses, goats, sheep, swine, cattle, llamas, and alpacas. Suitable primates include but are not limited to capuchin monkeys, chimpanzees, lemurs, macaques, marmosets, tamarins, spider monkeys, squirrel monkeys, and vervet monkeys. Non-limiting examples of birds include chickens, turkeys, ducks, and geese. Alternatively, the animal may be an invertebrate such as an insect, a nematode, and the like. Non-limiting examples of insects include Drosophila and mosquitoes. An exemplary animal is a rat. Non-limiting examples of suitable rat strains include Dahl Salt-Sensitive, Fischer 344, Lewis, Long Evans Hooded, Sprague-Dawley, and Wistar. In another iteration of the invention, the animal does not comprise a genetically modified mouse. In each of the foregoing iterations of suitable animals for the invention, the animal does not include exogenously introduced, randomly integrated transposon sequences.

### (c) Neurodevelopmental Protein

The neurodevelopmental protein may be from any of the animals listed above. Furthermore, the neurodevelopmental protein may be a human neurodevelopmental protein. Additionally, the neurodevelopmental protein may be a bacterial, fungal, or plant neurodevelopmental protein. The type of animal and the source of the protein can and will vary. The protein may be endogenous or exogenous (such as an orthologous protein). As an example, the genetically modified animal may be a rat, cat, dog, or pig, and the orthologous neurodevelopmental protein may be human. Alternatively, the genetically modified animal may be a rat, cat, or pig, and the orthologous neurodevelopmental protein may be canine. One of skill in the art will readily appreciate that numerous combinations are possible.

Additionally, the neurodevelopmental gene may be modified to include a tag or reporter gene or genes as are well-known. Reporter genes include those encoding selectable markers such as cloramphenicol acetyltransferase (CAT) and neomycin phosphotransferase (neo), and those encoding a fluorescent protein such as green fluorescent protein (GFP), red fluorescent protein, or any genetically engineered variant thereof that improves the reporter performance. Non-limiting examples of known such FP variants include EGFP, blue fluorescent protein (EBFP, EBFP2, Azurite, mKalama1), cyan fluorescent protein (ECFP, Cerulean, CyPet) and yellow fluorescent protein derivatives (YFP, Citrine, Venus, YPet). For example, in a genetic construct containing a reporter gene, the reporter gene sequence can be fused directly to the targeted gene to create a gene fusion. A reporter sequence can be integrated in a targeted manner in the targeted gene, for example the reporter sequences may be integrated specifically at the 5′ or 3′ end of the targeted gene. The two genes are thus under the control of the same promoter elements and are transcribed into a single messenger RNA molecule. Alternatively, the reporter gene may be used to monitor the activity of a promoter in a genetic construct, for example by placing the reporter sequence downstream of the target promoter such that expression of the reporter gene is under the control of the target promoter, and activity of the reporter gene can be directly and quantitatively measured, typically in comparison to activity observed under a strong consensus promoter. It will be understood that doing so may or may not lead to destruction of the targeted gene.

### (II) Genetically Modified Cells

A further aspect of the present disclosure provides genetically modified cells or cell lines comprising at least one edited chromosomal sequence encoding a neurodevelopmental protein. The genetically modified cell or cell line may be derived from any of the genetically modified animals disclosed herein. Alternatively, the chromosomal sequence coding a neurodevelopmental protein may be edited in a cell as detailed below. The disclosure also encompasses a lysate of said cells or cell lines.

In general, the cells will be eukaryotic cells. Suitable host cells include fungi or yeast, such as Pichia, Saccharomyces, or Schizosaccharomyces; insect cells, such as SF9 cells from Spodoptera frugiperda or S2 cells from Drosophila melanogaster; and animal cells, such as mouse, rat, hamster, non-human primate, or human cells. Exemplary cells are mammalian. The mammalian cells may be primary cells. In general, any primary cell that is sensitive to double strand breaks may be used. The cells may be of a variety of cell types, e.g., fibroblast, myoblast, T or B cell, macrophage, epithelial cell, and so forth.

When mammalian cell lines are used, the cell line may be any established cell line or a primary cell line that is not yet described. The cell line may be adherent or non-adherent, or the cell line may be grown under conditions that encourage adherent, non-adherent or organotypic growth using standard techniques known to individuals skilled in the art. Non-limiting examples of suitable mammalian cell lines include Chinese hamster ovary (CHO) cells, monkey kidney CVI line transformed by SV40 (COS7), human embryonic kidney line 293, baby hamster kidney cells (BHK), mouse sertoli cells (TM4), monkey kidney cells (CV1-76), African green monkey kidney cells (VERO), human cervical carcinoma cells (HeLa), canine kidney cells (MDCK), buffalo rat liver cells (BRL 3A), human lung cells (W138), human liver cells (Hep G2), mouse mammary tumor cells (MMT), rat hepatoma cells (HTC), HIH/3T3 cells, the human U2-OS osteosarcoma cell line, the human A549 cell line, the human K562 cell line, the human HEK293 cell lines, the human HEK293T cell line, and TR1 cells. For an extensive list of mammalian cell lines, those of ordinary skill in the art may refer to the American Type Culture Collection catalog (ATCC®, Mamassas, Va.).

In still other embodiments, the cell may be a stem cell. Suitable stem cells include without limit embryonic stem cells, ES-like stem cells, fetal stem cells, adult stem cells, pluripotent stem cells, induced pluripotent stem cells, multipotent stem cells, oligopotent stem cells, and unipotent stem cells.

### (III) Zinc Finger-Mediated Genome Editing

In general, the genetically modified animal or cell detailed above in sections (I) and (II), respectively, is generated using a zinc finger nuclease-mediated genome editing process. The process for editing a chromosomal sequence comprises: (a) introducing into an embryo or cell at least one nucleic acid encoding a zinc finger nuclease that recognizes a target sequence in the chromosomal sequence and is able to cleave a site in the chromosomal sequence, and, optionally, (i) at least one donor polynucleotide comprising a sequence for integration flanked by an upstream sequence and a downstream sequence that share substantial sequence identity with either side of the cleavage site, or (ii) at least one exchange polynucleotide comprising a sequence that is substantially identical to a portion of the chromosomal sequence at the cleavage site and which further comprises at least one nucleotide change; and (b) culturing the embryo or cell to allow expression of the zinc finger nuclease such that the zinc finger nuclease introduces a double-stranded break into the chromosomal sequence, and wherein the double-stranded break is repaired by (i) a non-homologous end-joining repair process such that an inactivating mutation is introduced into the chromosomal sequence, or (ii) a homology-directed repair process such that the sequence in the donor polynucleotide is integrated into the chromosomal sequence or the sequence in the exchange polynucleotide is exchanged with the portion of the chromosomal sequence.

Components of the zinc finger nuclease-mediated method are described in more detail below.

### (a) Zinc Finger Nuclease

The method comprises, in part, introducing into an embryo or cell at least one nucleic acid encoding a zinc finger nuclease. Typically, a zinc finger nuclease comprises a DNA binding domain (i.e., zinc finger) and a cleavage domain (i.e., nuclease). The DNA binding and cleavage domains are described below. The nucleic acid encoding a zinc finger nuclease may comprise DNA or RNA. For example, the nucleic acid encoding a zinc finger nuclease may comprise mRNA. When the nucleic acid encoding a zinc finger nuclease comprises mRNA, the mRNA molecule may be 5′ capped. Similarly, when the nucleic acid encoding a zinc finger nuclease comprises mRNA, the mRNA molecule may be polyadenylated. An exemplary nucleic acid according to the method is a capped and polyadenylated mRNA molecule encoding a zinc finger nuclease. Methods for capping and polyadenylating mRNA are known in the art.

(i) Zinc Finger Binding Domain

Zinc finger binding domains may be engineered to recognize and bind to any nucleic acid sequence of choice. See, for example, Beerli et al. (2002) Nat. Biotechnol. 20:135-141; Pabo et al. (2001) Ann. Rev. Biochem. 70:313-340; Isalan et al. (2001) Nat. Biotechnol. 19:656-660; Segal et al. (2001) Curr. Opin. Biotechnol. 12:632-637; Choo et al. (2000) Curr. Opin. Struct. Biol. 10:411-416; Zhang et al. (2000) J. Biol. Chem. 275(43):33850-33860; Doyon et al. (2008) Nat. Biotechnol. 26:702-708; and Santiago et al. (2008) Proc. Natl. Acad. Sci. USA 105:5809-5814. An engineered zinc finger binding domain may have a novel binding specificity compared to a naturally-occurring zinc finger protein. Engineering methods include, but are not limited to, rational design and various types of selection. Rational design includes, for example, using databases comprising doublet, triplet, and/or quadruplet nucleotide sequences and individual zinc finger amino acid sequences, in which each doublet, triplet or quadruplet nucleotide sequence is associated with one or more amino acid sequences of zinc fingers which bind the particular triplet or quadruplet sequence. See, for example, U.S. Pat. Nos. 6,453,242 and 6,534,261, the disclosures of which are incorporated by reference herein in their entireties. As an example, the algorithm of described in U.S. Pat. No. 6,453,242 may be used to design a zinc finger binding domain to target a preselected sequence. Alternative methods, such as rational design using a nondegenerate recognition code table may also be used to design a zinc finger binding domain to target a specific sequence (Sera et al. (2002) Biochemistry 41:7074-7081). Publically available web-based tools for identifying potential target sites in DNA sequences and designing zinc finger binding domains may be found at http://www.zincfingertools.org and http://bindr.gdcb.iastate.edu/ZiFiT/, respectively (Mandell et al. (2006) Nuc. Acid Res. 34:W516-W523; Sander et al. (2007) Nuc. Acid Res. 35:W599-W605).

A zinc finger binding domain may be designed to recognize a DNA sequence ranging from about 3 nucleotides to about 21 nucleotides in length, or from about 8 to about 19 nucleotides in length. In general, the zinc finger binding domains of the zinc finger nucleases disclosed herein comprise at least three zinc finger recognition regions (i.e., zinc fingers). In one embodiment, the zinc finger binding domain may comprise four zinc finger recognition regions. In another embodiment, the zinc finger binding domain may comprise five zinc finger recognition regions. In still another embodiment, the zinc finger binding domain may comprise six zinc finger recognition regions. A zinc finger binding domain may be designed to bind to any suitable target DNA sequence. See for example, U.S. Pat. Nos. 6,607,882; 6,534,261 and 6,453,242, the disclosures of which are incorporated by reference herein in their entireties.

Exemplary methods of selecting a zinc finger recognition region may include phage display and two-hybrid systems, and are disclosed in U.S. Pat. Nos. 5,789,538; 5,925,523; 6,007,988; 6,013,453; 6,410,248; 6,140,466; 6,200,759; and 6,242,568; as well as WO 98/37186; WO 98/53057; WO 00/27878; WO 01/88197 and GB 2,338,237, each of which is incorporated by reference herein in its entirety. In addition, enhancement of binding specificity for zinc finger binding domains has been described, for example, in WO 02/077227.

Zinc finger binding domains and methods for design and construction of fusion proteins (and polynucleotides encoding same) are known to those of skill in the art and are described in detail in U.S. Patent Application Publication Nos. 20050064474 and 20060188987, each incorporated by reference herein in its entirety. Zinc finger recognition regions and/or multi-fingered zinc finger proteins may be linked together using suitable linker sequences, including for example, linkers of five or more amino acids in length. See, U.S. Pat. Nos. 6,479,626; 6,903,185; and 7,153,949, the disclosures of which are incorporated by reference herein in their entireties, for non-limiting examples of linker sequences of six or more amino acids in length. The zinc finger binding domain described herein may include a combination of suitable linkers between the individual zinc fingers of the protein.

In some embodiments, the zinc finger nuclease may further comprise a nuclear localization signal or sequence (NLS). A NLS is an amino acid sequence which facilitates targeting the zinc finger nuclease protein into the nucleus to introduce a double stranded break at the target sequence in the chromosome. Nuclear localization signals are known in the art. See, for example, Makkerh et al. (1996) Current Biology 6:1025-1027.

(ii) Cleavage Domain

A zinc finger nuclease also includes a cleavage domain. The cleavage domain portion of the zinc finger nucleases disclosed herein may be obtained from any endonuclease or exonuclease. Non-limiting examples of endonucleases from which a cleavage domain may be derived include, but are not limited to, restriction endonucleases and homing endonucleases. See, for example, 2002-2003 Catalog, New England Biolabs, Beverly, Mass.; and Belfort et al. (1997) Nucleic Acids Res. 25:3379-3388 or www.neb.com. Additional enzymes that cleave DNA are known (e.g., 51 Nuclease; mung bean nuclease; pancreatic DNase I; micrococcal nuclease; yeast HO endonuclease). See also Linn et al. (eds.) Nucleases, Cold Spring Harbor Laboratory Press, 1993. One or more of these enzymes (or functional fragments thereof) may be used as a source of cleavage domains.

A cleavage domain also may be derived from an enzyme or portion thereof, as described above, that requires dimerization for cleavage activity. Two zinc finger nucleases may be required for cleavage, as each nuclease comprises a monomer of the active enzyme dimer. Alternatively, a single zinc finger nuclease may comprise both monomers to create an active enzyme dimer. As used herein, an “active enzyme dimer” is an enzyme dimer capable of cleaving a nucleic acid molecule. The two cleavage monomers may be derived from the same endonuclease (or functional fragments thereof), or each monomer may be derived from a different endonuclease (or functional fragments thereof).

When two cleavage monomers are used to form an active enzyme dimer, the recognition sites for the two zinc finger nucleases are preferably disposed such that binding of the two zinc finger nucleases to their respective recognition sites places the cleavage monomers in a spatial orientation to each other that allows the cleavage monomers to form an active enzyme dimer, e.g., by dimerizing. As a result, the near edges of the recognition sites may be separated by about 5 to about 18 nucleotides. For instance, the near edges may be separated by about 5, 6, 7, 8, 9, 10, 11, 12, 13, 14, 15, 16, 17 or 18 nucleotides. It will however be understood that any integral number of nucleotides or nucleotide pairs may intervene between two recognition sites (e.g., from about 2 to about 50 nucleotide pairs or more). The near edges of the recognition sites of the zinc finger nucleases, such as for example those described in detail herein, may be separated by 6 nucleotides. In general, the site of cleavage lies between the recognition sites.

Restriction endonucleases (restriction enzymes) are present in many species and are capable of sequence-specific binding to DNA (at a recognition site), and cleaving DNA at or near the site of binding. Certain restriction enzymes (e.g., Type IIS) cleave DNA at sites removed from the recognition site and have separable binding and cleavage domains. For example, the Type IIS enzyme Fok I catalyzes double-stranded cleavage of DNA, at 9 nucleotides from its recognition site on one strand and 13 nucleotides from its recognition site on the other. See, for example, U.S. Pat. Nos. 5,356,802; 5,436,150 and 5,487,994; as well as Li et al. (1992) Proc. Natl. Acad. Sci. USA 89:4275-4279; Li et al. (1993) Proc. Natl. Acad. Sci. USA 90:2764-2768; Kim et al. (1994a) Proc. Natl. Acad. Sci. USA 91:883-887; Kim et al. (1994b) J. Biol. Chem. 269:31, 978-31, 982. Thus, a zinc finger nuclease may comprise the cleavage domain from at least one Type IIS restriction enzyme and one or more zinc finger binding domains, which may or may not be engineered. Exemplary Type IIS restriction enzymes are described for example in International Publication WO 07/014,275, the disclosure of which is incorporated by reference herein in its entirety. Additional restriction enzymes also contain separable binding and cleavage domains, and these also are contemplated by the present disclosure. See, for example, Roberts et al. (2003) Nucleic Acids Res. 31:418-420.

An exemplary Type IIS restriction enzyme, whose cleavage domain is separable from the binding domain, is Fok I. This particular enzyme is active as a dimmer (Bitinaite et al. (1998) Proc. Natl. Acad. Sci. USA 95: 10, 570-10, 575). Accordingly, for the purposes of the present disclosure, the portion of the Fok I enzyme used in a zinc finger nuclease is considered a cleavage monomer. Thus, for targeted double-stranded cleavage using a Fok I cleavage domain, two zinc finger nucleases, each comprising a FokI cleavage monomer, may be used to reconstitute an active enzyme dimer. Alternatively, a single polypeptide molecule containing a zinc finger binding domain and two Fok I cleavage monomers may also be used.

In certain embodiments, the cleavage domain may comprise one or more engineered cleavage monomers that minimize or prevent homodimerization, as described, for example, in U.S. Patent Publication Nos. 20050064474, 20060188987, and 20080131962, each of which is incorporated by reference herein in its entirety. By way of non-limiting example, amino acid residues at positions 446, 447, 479, 483, 484, 486, 487, 490, 491, 496, 498, 499, 500, 531, 534, 537, and 538 of Fok I are all targets for influencing dimerization of the Fok I cleavage half-domains. Exemplary engineered cleavage monomers of Fok I that form obligate heterodimers include a pair in which a first cleavage monomer includes mutations at amino acid residue positions 490 and 538 of Fok I and a second cleavage monomer that includes mutations at amino-acid residue positions 486 and 499.

Thus, in one embodiment, a mutation at amino acid position 490 replaces Glu (E) with Lys (K); a mutation at amino acid residue 538 replaces Iso (I) with Lys (K); a mutation at amino acid residue 486 replaces Gln (O) with Glu (E); and a mutation at position 499 replaces Iso (I) with Lys (K). Specifically, the engineered cleavage monomers may be prepared by mutating positions 490 from E to K and 538 from Ito K in one cleavage monomer to produce an engineered cleavage monomer designated “E490K:1538K” and by mutating positions 486 from Q to E and 499 from Ito L in another cleavage monomer to produce an engineered cleavage monomer designated “Q486E:1499L.” The above described engineered cleavage monomers are obligate heterodimer mutants in which aberrant cleavage is minimized or abolished. Engineered cleavage monomers may be prepared using a suitable method, for example, by site-directed mutagenesis of wild-type cleavage monomers (Fok I) as described in U.S. Patent Publication No. 20050064474 (see Example 5).

The zinc finger nuclease described above may be engineered to introduce a double stranded break at the targeted site of integration. The double stranded break may be at the targeted site of integration, or it may be up to 1, 2, 3, 4, 5, 10, 15, 20, 25, 30, 35, 40, 45, 50, 100, or 1000 nucleotides away from the site of integration. In some embodiments, the double stranded break may be up to 1, 2, 3, 4, 5, 10, 15, or 20 nucleotides away from the site of integration. In other embodiments, the double stranded break may be up to 10, 15, 20, 25, 30, 35, 40, 45, or 50 nucleotides away from the site of integration. In yet other embodiments, the double stranded break may be up to 50, 100, or 1000 nucleotides away from the site of integration.

### (b) Optional Donor Polynucleotide

The method for editing chromosomal sequences encoding neurodevelopmental proteins may further comprise introducing at least one donor polynucleotide comprising a sequence encoding a neurodevelopmental protein into the embryo or cell. A donor polynucleotide comprises at least three components: the sequence coding the neurodevelopmental protein, an upstream sequence, and a downstream sequence. The sequence encoding the protein is flanked by the upstream and downstream sequence, wherein the upstream and downstream sequences share sequence similarity with either side of the site of integration in the chromosome.

Typically, the donor polynucleotide will be DNA. The donor polynucleotide may be a DNA plasmid, a bacterial artificial chromosome (BAC), a yeast artificial chromosome (YAC), a viral vector, a linear piece of DNA, a PCR fragment, a naked nucleic acid, or a nucleic acid complexed with a delivery vehicle such as a liposome or poloxamer. An exemplary donor polynucleotide comprising the sequence encoding a neurodevelopmental protein may be a BAC.

The sequence of the donor polynucleotide that encodes the neurodevelopmental protein may include coding (i.e., exon) sequence, as well as intron sequences and upstream regulatory sequences (such as, e.g., a promoter). Depending upon the identity and the source of the neurodevelopmental protein, the size of the sequence encoding the neurodevelopmental protein can and will vary. For example, the sequence encoding the neurodevelopmental protein may range in size from about 1 kb to about 5,000 kb.

The donor polynucleotide also comprises upstream and downstream sequence flanking the sequence encoding the neurodevelopmental protein. The upstream and downstream sequences in the donor polynucleotide are selected to promote recombination between the chromosomal sequence of interest and the donor polynucleotide. The upstream sequence, as used herein, refers to a nucleic acid sequence that shares sequence similarity with the chromosomal sequence upstream of the targeted site of integration. Similarly, the downstream sequence refers to a nucleic acid sequence that shares sequence similarity with the chromosomal sequence downstream of the targeted site of integration. The upstream and downstream sequences in the donor polynucleotide may share about 75%, 80%, 85%, 90%, 95%, or 100% sequence identity with the targeted chromosomal sequence. In other embodiments, the upstream and downstream sequences in the donor polynucleotide may share about 95%, 96%, 97%, 98%, 99%, or 100% sequence identity with the targeted chromosomal sequence. In an exemplary embodiment, the upstream and downstream sequences in the donor polynucleotide may share about 99% or 100% sequence identity with the targeted chromosomal sequence.

An upstream or downstream sequence may comprise from about 50 bp to about 2500 bp. In one embodiment, an upstream or downstream sequence may comprise about 100, 200, 300, 400, 500, 600, 700, 800, 900, 1000, 1100, 1200, 1300, 1400, 1500, 1600, 1700, 1800, 1900, 2000, 2100, 2200, 2300, 2400, or 2500 bp. An exemplary upstream or downstream sequence may comprise about 200 bp to about 2000 bp, about 600 bp to about 1000 bp, or more particularly about 700 bp to about 1000 bp.

In some embodiments, the donor polynucleotide may further comprise a marker. Such a marker may make it easy to screen for targeted integrations. Non-limiting examples of suitable markers include restriction sites, fluorescent proteins, or selectable markers.

One of skill in the art would be able to construct a donor polynucleotide as described herein using well-known standard recombinant techniques (see, for example, Sambrook et al., 2001 and Ausubel et al., 1996).

In the method detailed above for integrating a sequence encoding the neurodevelopmental protein, a double stranded break introduced into the chromosomal sequence by the zinc finger nuclease is repaired, via homologous recombination with the donor polynucleotide, such that the sequence encoding the neurodevelopmental protein is integrated into the chromosome. The presence of a double-stranded break facilitates integration of the sequence into the chromosome. A donor polynucleotide may be physically integrated or, alternatively, the donor polynucleotide may be used as a template for repair of the break, resulting in the introduction of the sequence encoding the neurodevelopmental protein as well as all or part of the upstream and downstream sequences of the donor polynucleotide into the chromosome. Thus, endogenous chromosomal sequence may be converted to the sequence of the donor polynucleotide.

### (c) Optional Exchange Polynucleotide

The method for editing chromosomal sequences encoding neurodevelopmental proteins may further comprise introducing into the embryo or cell at least one exchange polynucleotide comprising a sequence that is substantially identical to the chromosomal sequence at the site of cleavage and which further comprises at least one specific nucleotide change.

Typically, the exchange polynucleotide will be DNA. The exchange polynucleotide may be a DNA plasmid, a bacterial artificial chromosome (BAC), a yeast artificial chromosome (YAC), a viral vector, a linear piece of DNA, a PCR fragment, a naked nucleic acid, or a nucleic acid complexed with a delivery vehicle such as a liposome or poloxamer. An exemplary exchange polynucleotide may be a DNA plasmid.

The sequence in the exchange polynucleotide is substantially identical to a portion of the chromosomal sequence at the site of cleavage. In general, the sequence of the exchange polynucleotide will share enough sequence identity with the chromosomal sequence such that the two sequences may be exchanged by homologous recombination. For example, the sequence in the exchange polynucleotide may have at least about 80, 81, 82, 83, 84, 85, 86, 87, 88, 89, 90, 91, 92, 93, 94, 95, 96, 97, 98, or 99% sequence identity with a portion of the chromosomal sequence.

Importantly, the sequence in the exchange polynucleotide comprises at least one specific nucleotide change with respect to the sequence of the corresponding chromosomal sequence. For example, one nucleotide in a specific codon may be changed to another nucleotide such that the codon codes for a different amino acid. In one embodiment, the sequence in the exchange polynucleotide may comprise one specific nucleotide change such that the encoded protein comprises one amino acid change. In other embodiments, the sequence in the exchange polynucleotide may comprise two, three, four, or more specific nucleotide changes such that the encoded protein comprises one, two, three, four, or more amino acid changes. In still other embodiments, the sequence in the exchange polynucleotide may comprise a three nucleotide deletion or insertion such that the reading frame of the coding reading is not altered (and a functional protein is produced). The expressed protein, however, would comprise a single amino acid deletion or insertion.

The length of the sequence in the exchange polynucleotide that is substantially identical to a portion of the chromosomal sequence at the site of cleavage can and will vary. In general, the sequence in the exchange polynucleotide may range from about 50 bp to about 10,000 bp in length. In various embodiments, the sequence in the exchange polynucleotide may be about 100, 200, 400, 600, 800, 1000, 1200, 1400, 1600, 1800, 2000, 2200, 2400, 2600, 2800, 3000, 3200, 3400, 3600, 3800, 4000, 4200, 4400, 4600, 4800, or 5000 bp in length. In other embodiments, the sequence in the exchange polynucleotide may be about 5500, 6000, 6500, 6000, 6500, 7000, 7500, 8000, 8500, 9000, 9500, or 10,000 bp in length. One of skill in the art would be able to construct an exchange polynucleotide as described herein using well-known standard recombinant techniques (see, for example, Sambrook et al., 2001 and Ausubel et al., 1996).

In the method detailed above for modifying a chromosomal sequence, a double stranded break introduced into the chromosomal sequence by the zinc finger nuclease is repaired, via homologous recombination with the exchange polynucleotide, such that the sequence in the exchange polynucleotide may be exchanged with a portion of the chromosomal sequence. The presence of the double stranded break facilitates homologous recombination and repair of the break. The exchange polynucleotide may be physically integrated or, alternatively, the exchange polynucleotide may be used as a template for repair of the break, resulting in the exchange of the sequence information in the exchange polynucleotide with the sequence information in that portion of the chromosomal sequence. Thus, a portion of the endogenous chromosomal sequence may be converted to the sequence of the exchange polynucleotide. The changed nucleotide(s) may be at or near the site of cleavage. Alternatively, the changed nucleotide(s) may be anywhere in the exchanged sequences. As a consequence of the exchange, however, the chromosomal sequence is modified.

### (d) Delivery of Nucleic Acids

To mediate zinc finger nuclease genomic editing, at least one nucleic acid molecule encoding a zinc finger nuclease and, optionally, at least one exchange polynucleotide or at least one donor polynucleotide are delivered to the embryo or the cell of interest. Typically, the embryo is a fertilized one-cell stage embryo of the species of interest.

Suitable methods of introducing the nucleic acids to the embryo or cell include microinjection, electroporation, sonoporation, biolistics, calcium phosphate-mediated transfection, cationic transfection, liposome transfection, dendrimer transfection, heat shock transfection, nucleofection transfection, magnetofection, lipofection, impalefection, optical transfection, proprietary agent-enhanced uptake of nucleic acids, and delivery via liposomes, immunoliposomes, virosomes, or artificial virions. In one embodiment, the nucleic acids may be introduced into an embryo by microinjection. The nucleic acids may be microinjected into the nucleus or the cytoplasm of the embryo. In another embodiment, the nucleic acids may be introduced into a cell by nucleofection.

In embodiments in which both a nucleic acid encoding a zinc finger nuclease and a donor (or exchange) polynucleotide are introduced into an embryo or cell, the ratio of donor (or exchange) polynucleotide to nucleic acid encoding a zinc finger nuclease may range from about 1:10 to about 10:1. In various embodiments, the ratio of donor (or exchange) polynucleotide to nucleic acid encoding a zinc finger nuclease may be about 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, or 10:1. In one embodiment, the ratio may be about 1:1.

In embodiments in which more than one nucleic acid encoding a zinc finger nuclease and, optionally, more than one donor (or exchange) polynucleotide are introduced into an embryo or cell, the nucleic acids may be introduced simultaneously or sequentially. For example, nucleic acids encoding the zinc finger nucleases, each specific for a distinct recognition sequence, as well as the optional donor (or exchange) polynucleotides, may be introduced at the same time. Alternatively, each nucleic acid encoding a zinc finger nuclease, as well as the optional donor (or exchange) polynucleotides, may be introduced sequentially.

### (e) Culturing the Embryo or Cell

The method of inducing genomic editing with a zinc finger nuclease further comprises culturing the embryo or cell comprising the introduced nucleic acid(s) to allow expression of the zinc finger nuclease. An embryo may be cultured in vitro (e.g., in cell culture). Typically, the embryo is cultured at an appropriate temperature and in appropriate media with the necessary O₂/CO₂ ratio to allow the expression of the zinc finger nuclease. Suitable non-limiting examples of media include M2, M16, KSOM, BMOC, and HTF media. A skilled artisan will appreciate that culture conditions can and will vary depending on the species of embryo. Routine optimization may be used, in all cases, to determine the best culture conditions for a particular species of embryo. In some cases, a cell line may be derived from an in vitro-cultured embryo (e.g., an embryonic stem cell line).

Alternatively, an embryo may be cultured in vivo by transferring the embryo into the uterus of a female host. Generally speaking the female host is from the same or similar species as the embryo. Preferably, the female host is pseudo-pregnant. Methods of preparing pseudo-pregnant female hosts are known in the art. Additionally, methods of transferring an embryo into a female host are known. Culturing an embryo in vivo permits the embryo to develop and may result in a live birth of an animal derived from the embryo. Such an animal would comprise the edited chromosomal sequence encoding the neurodevelopmental protein in every cell of the body.

Similarly, cells comprising the introduced nucleic acids may be cultured using standard procedures to allow expression of the zinc finger nuclease. Standard cell culture techniques are described, for example, in Santiago et al. (2008) PNAS 105:5809-5814; Moehle et al. (2007) PNAS 104:3055-3060; Urnov et al. (2005) Nature 435:646-651; and Lombardo et al (2007) Nat. Biotechnology 25:1298-1306. Those of skill in the art appreciate that methods for culturing cells are known in the art and can and will vary depending on the cell type. Routine optimization may be used, in all cases, to determine the best techniques for a particular cell type.

Upon expression of the zinc finger nuclease, the chromosomal sequence may be edited. In cases in which the embryo or cell comprises an expressed zinc finger nuclease but no donor (or exchange) polynucleotide, the zinc finger nuclease recognizes, binds, and cleaves the target sequence in the chromosomal sequence of interest. The double-stranded break introduced by the zinc finger nuclease is repaired by an error-prone non-homologous end-joining DNA repair process. Consequently, a deletion, insertion, or nonsense mutation may be introduced in the chromosomal sequence such that the sequence is inactivated.

In cases in which the embryo or cell comprises an expressed zinc finger nuclease as well as a donor (or exchange) polynucleotide, the zinc finger nuclease recognizes, binds, and cleaves the target sequence in the chromosome. The double-stranded break introduced by the zinc finger nuclease is repaired, via homologous recombination with the donor (or exchange) polynucleotide, such that the sequence in the donor polynucleotide is integrated into the chromosomal sequence (or a portion of the chromosomal sequence is converted to the sequence in the exchange polynucleotide). As a consequence, a sequence may be integrated into the chromosomal sequence (or a portion of the chromosomal sequence may be modified).

The genetically modified animals disclosed herein may be crossbred to create animals comprising more than one edited chromosomal sequence or to create animals that are homozygous for one or more edited chromosomal sequences. For example, two animals comprising the same edited chromosomal sequence may be crossbred to create an animal homozygous for the edited chromosomal sequence. Alternatively, animals with different edited chromosomal sequences may be crossbred to create an animal comprising both edited chromosomal sequences.

For example, animal A comprising an inactivated bmp4 chromosomal sequence may be crossed with animal B comprising a chromosomally integrated sequence encoding a human BMP4 protein to give rise to a “humanized” BMP4 offspring comprising both the inactivated bmp4 chromosomal sequence and the chromosomally integrated human BMP4 sequence. Similarly, an animal comprising an inactivated bmp4 chrd chromosomal sequence may be crossed with an animal comprising a chromosomally integrated sequence encoding the human neurodevelopmental CHRD protein to generate “humanized” neurodevelopmental CHRD offspring. Moreover, a humanized BMP4 animal may be crossed with a humanized CHRD animal to create a humanized BMP4/CHRD offspring. Those of skill in the art will appreciate that many combinations are possible. Exemplary combinations of chromosomal sequences are presented above.

In other embodiments, an animal comprising an edited chromosomal sequence disclosed herein may be crossbred to combine the edited chromosomal sequence with other genetic backgrounds. By way of non-limiting example, other genetic backgrounds may include wild-type genetic backgrounds, genetic backgrounds with deletion mutations, genetic backgrounds with another targeted integration, and genetic backgrounds with non-targeted integrations. Suitable integrations may include without limit nucleic acids encoding drug transporter proteins, Mdr protein, and the like.

### (IV) Applications

A further aspect of the present disclosure encompasses a method for assessing an effect of an agent such as a pharmaceutically active ingredient, a drug, a toxin, or a chemical. For example, the effect of an agent may be measured in a “humanized” genetically modified animal, such that the information gained therefrom may be used to predict the effect of the agent in a human. In general, the method comprises administering the agent to a genetically modified animal comprising at least one inactivated chromosomal sequence encoding a neurodevelopmental protein and at least one chromosomally integrated sequence encoding an orthologous neurodevelopmental protein, and comparing a parameter obtained from the genetically modified animal to the parameter obtained from a wild-type animal administered the same agent.

Suitable agents include without limit pharmaceutically active ingredients, drugs, foods, food additives, pesticides, herbicides, toxins, industrial chemicals, household chemicals, and other environmental chemicals. The agent may be a therapeutic treatment for a neurodevelopmental disorder, including but not limited to administering of one or more novel candidate therapeutic compounds, administering a novel combination of established therapeutic compounds, a novel therapeutic method, and any combination thereof. Non-limiting examples of novel therapeutic methods include various drug delivery mechanisms such as oral or injected therapeutic compositions, drug-releasing implants, nanotechnology applications in drug therapy, vaccine compositions, surgery, and combinations thereof.

Non-limiting examples of suitable parameters for the assessment of the agent include: (a) rate of elimination of the agent or at least one agent metabolite; (b) circulatory levels of the agent or at least one agent metabolite; (c) bioavailability of the agent or at least one agent metabolite; (d) rate of metabolism of the agent or at least one agent metabolite; (e) rate of clearance of the agent or at least one agent metabolite; (f) toxicity of the agent or at least one agent metabolite; (g) efficacy of the agent or at least one agent metabolite; (h) disposition of the agent or at least one agent metabolite; and (i) extrahepatic contribution to metabolic rate and clearance of the agent or at least one agent metabolite; and (j) ability of the agent to modify an incidence or indication of a neurodevelopmental disorder in the genetically modified animal.

For example, an ADME-Tox profile of an agent may be assessed using the genetically modified animal. The ADME-Tox profile may include assessments of at least one or more physiologic and metabolic consequences of administering the agent. In addition, the ADME-Tox profile may assess behavioral effects such as addiction or depression in response to the agent.

The incidence or indication of the neurodevelopmental disorder may occur spontaneously in the genetically modified animal. Alternatively, the incidence or indication of the neurodevelopmental disorder may be promoted by exposure to a neurodisruptive agent. Non-limiting examples of neurodisruptive agents include a neurodevelopmental protein such as any of those described above, a drug, a toxin, a chemical, and an environmental stress. Non-limiting examples of environmental stresses include forced swimming, cold swimming, platform shaker stimuli, loud noises, and immobilization stress.

Non-limiting examples of neurodevelopmental disorders include autism spectrum disorders such as autism, Asperger syndrome, and Pediatric Autoimmune Neuropsychiatric Disorders Associated with Streptococcal infections (PANDAS); Rett syndrome; Williams syndrome; Renpenning's syndrome; fragile X syndrome; Down syndrome; Prader-Willi syndrome; Sotos syndrome; Tuberous sclerosis complex (TSC); Timothy syndrome; Joubert syndrome; holoprosencephaly; Hirschsprung's disease; intestinal neuronal dysplasia; and Williams syndrome.

Suitable neurodevelopmental proteins may include any one or more of neurodevelopmental proteins described above, including but not limited to BMP4, CHRD, NOG, WNT2, WNT2B, WNT3A, WNT4, WNT5A, WNT6, WNT7B, WNT8B, WNT9A, WNT9B, WNT10A, WNT10B, WNT16, OTX2, GBX2, FGF8, RELN, DAB1, POU4F1, NUMB, and any combination thereof.

Yet another aspect encompasses a method for assessing the therapeutic potential of an agent as a treatment for a neurodevelopmental disorder. The method includes administering the agent to a genetically modified animal and comparing a selected parameter obtained from the genetically modified animal to the selected parameter obtained from a wild-type animal with no exposure to the same agent. The genetically modified animal comprises at least one edited chromosomal sequence encoding a neurodevelopmental protein.

The selected parameter may be chosen from a) spontaneous behaviors; b) performance during behavioral testing; c) physiological anomalies; d) abnormalities in tissues or cells; e) biochemical function; and f) molecular structures. These selected parameters may also be used to assess a genetically modified animal for one or more indications of a neurodevelopmental disorder. As described previously, the genetically modified animal may develop the neurodevelopmental disorder spontaneously, or the development of the disorder may be promoted by a neurodisruptive agent.

Spontaneous behavior may be assessed using any one or more methods of spontaneous behavioral observation known in the art. In general, any spontaneous behavior within a known behavioral repertoire of an animal may be observed, including movement, posture, social interaction, rearing, sleeping, blinking, eating, drinking, urinating, defecating, mating, and aggression. An extensive battery of observations for quantifying the spontaneous behavior of mice and rats is well-known in the art, including but not limited to home-cage observations such as body position, respiration, tonic involuntary movement, unusual motor behavior such as pacing or rocking, catatonic behavior, vocalization, palpebral closure, mating frequency, running wheel behavior, nest building, and frequency of aggressive interactions.

Performance during behavioral testing may be assessed using any number of behavioral tests known in the art. The particular type of performance test may depend upon at least one of several factors including the behavioral repertoire of the animal and the purpose of the testing. Non-limiting examples of tests for assessing the reflex function of rats include assessments of approach response, touch response, eyelid reflex, pinna reflex, sound response, tail pinch response, pupillary reflex, and righting reflex. Non-limiting examples of behavioral tests suitable for assessing the motor function of rats includes open field locomotor activity assessment, the rotarod test, the grip strength test, the cylinder test, the limb-placement or grid walk test, the vertical pole test, the Inverted grid test, the adhesive removal test, the painted paw or catwalk (gait) tests, the beam traversal test, and the inclined plane test. Non-limiting examples of behavioral tests suitable for assessing the long-term memory function of rats include the elevated plus maze test, the Morris water maze swim test, contextual fear conditioning, the Y-maze test, the T-maze test, the novel object recognition test, the active avoidance test, the passive (inhibitory) avoidance test, the radial arm maze test, the two-choice swim test, the hole board test, the olfactory discrimination (go-no-go) test, and the pre-pulse inhibition test. Non-limiting examples of behavioral tests suitable for assessing the anxiety of rats include the open field locomotion assessment, observations of marble-burying behavior, the elevated plus maze test, the light/dark box test. Non-limiting examples of behavioral tests suitable for assessing the depression of rats includes the forced swim test, the tail suspension test, the hot plate test, the tail suspension test, anhedonia observations, and the novelty suppressed feeding test.

Physiological anomalies may include any difference in physiological function between a genetically modified animal and a wild-type animal. Non-limiting examples of physiological functions include homeostasis, metabolism, sensory function, neurological function, musculoskeletal function, cardiovascular function, respiratory function, dermatological function, renal function, reproductive functions, immunological function, and endocrinological function. Numerous measures of physiological function are well-known in the art.

Abnormalities in tissues or cells may include any difference in the structure or function of a tissue or cell of a genetically modified animal and the corresponding structure or function of a wild-type animal. Non-limiting examples of cell or tissue abnormalities include cell hypertrophy, tissue hyperplasia, neoplasia, hypoplasia, aplasia, hypotrophy, dysplasia, overproduction or underproduction of cell products, abnormal neuronal discharge frequency, and changes in synaptic density of neurons.

Non-limiting examples of biochemical functions may include enzyme function, cell signaling function, maintenance of homeostasis, cellular respiration; methods of assessing biochemical functions are well known in the art. Molecular structures may be assessed using any method known in the art including microscopy such as dual-photon microscopy and scanning electron microscopy, and immunohistological techniques such as Western blot and ELISA.

A additional aspect provides a method for assessing a side effect of a therapeutic compound comprising administering the therapeutic compound to an animal model and assessing at least one or more behaviors chosen from learning, memory, anxiety, depression, addiction, sensory-motor function, taste preference, and odor preference. The animal model may be chosen from a genetically modified animal and a wild-type animal. The genetically modified animal comprises at least one edited chromosomal sequence encoding a neurodevelopmental protein. The therapeutic compound is chosen from a novel therapeutic compound and a novel combination of known therapeutic agents. Any of the methods described above to measure spontaneous behavior or performance during behavioral tests may be used to assess the side effect.

In this method, the therapeutic compound may be self-administered, or the therapeutic compound may be administered by another. The animal model may be contacted with the therapeutic compound using administration methods including oral ingestion, epidermal absorption, injection, absorption through the mucous membranes of the oral cavity, rectum, nasal cavity, lungs, or vagina, and any other suitable administration method known in the art. If the therapeutic compound is administered using oral ingestion, the therapeutic compound may be incorporated in an amount of water, food, or supplemental material such as a chewable or lickable object and provided to the animal model.

Also provided are methods to assess an effect of an agent in an isolated cell comprising at least one edited chromosomal sequence encoding a neurodevelopmental protein, as well as methods of using lysates of such cells (or cells derived from a genetically modified animal disclosed herein) to assess the effect of an agent. For example, the role of a particular neurodevelopmental protein in the metabolism of a particular agent may be determined using such methods. Similarly, substrate specificity and pharmacokinetic parameter may be readily determined using such methods. Those of skill in the art are familiar with suitable tests and/or procedures.

Yet another aspect encompasses a method for assessing the therapeutic efficacy of a potential gene therapy strategy. That is, a chromosomal sequence encoding a neurodevelopmental protein may be modified such that the incidence or indications of a neurodevelopmental disorder of a genetically modified animal are reduced or eliminated. In particular, the method comprises editing a chromosomal sequence encoding a neurodevelopmental protein such that an altered protein product is produced. The genetically modified animal may be exposed to a neurodisruptive agent described above and behavioral, cellular, and/or molecular responses may be measured and compared to those of a wild-type animal exposed to the same neurodisruptive agent. Consequently, the therapeutic potential of the neurodevelopmental gene therapy regime may be assessed.

Still yet another aspect encompasses a method of generating a cell line or cell lysate using a genetically modified animal comprising an edited chromosomal sequence encoding a neurodevelopmental protein. An additional other aspect encompasses a method of producing purified biological components using a genetically modified cell or animal comprising an edited chromosomal sequence encoding a neurodevelopmental protein. Non-limiting examples of biological components include antibodies, cytokines, signal proteins, enzymes, receptor agonists and receptor antagonists.

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

A “gene,” as used herein, refers to a DNA region (including exons and introns) encoding a gene product, as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites, and locus control regions.

The terms “nucleic acid” and “polynucleotide” refer to a deoxyribonucleotide or ribonucleotide polymer, in linear or circular conformation, and in either single- or double-stranded form. For the purposes of the present disclosure, these terms are not to be construed as limiting with respect to the length of a polymer. The terms can encompass known analogs of natural nucleotides, as well as nucleotides that are modified in the base, sugar and/or phosphate moieties (e.g., phosphorothioate backbones). In general, an analog of a particular nucleotide has the same base-pairing specificity; i.e., an analog of A will base-pair with T.

The terms “polypeptide” and “protein” are used interchangeably to refer to a polymer of amino acid residues.

The term “recombination” refers to a process of exchange of genetic information between two polynucleotides. For the purposes of this disclosure, “homologous recombination” refers to the specialized form of such exchange that takes place, for example, during repair of double-strand breaks in cells. This process requires sequence similarity between the two polynucleotides, uses a “donor” or “exchange” molecule to template repair of a “target” molecule (i.e., the one that experienced the double-strand break), and is variously known as “non-crossover gene conversion” or “short tract gene conversion,” because it leads to the transfer of genetic information from the donor to the target. Without being bound by any particular theory, such transfer can involve mismatch correction of heteroduplex DNA that forms between the broken target and the donor, and/or “synthesis-dependent strand annealing,” in which the donor is used to resynthesize genetic information that will become part of the target, and/or related processes. Such specialized homologous recombination often results in an alteration of the sequence of the target molecule such that part or all of the sequence of the donor polynucleotide is incorporated into the target polynucleotide.

As used herein, the terms “target site” or “target sequence” refer to a nucleic acid sequence that defines a portion of a chromosomal sequence to be edited and to which a zinc finger nuclease is engineered to recognize and bind, provided sufficient conditions for binding exist.

Techniques for determining nucleic acid and amino acid sequence identity are known in the art. Typically, such techniques include determining the nucleotide sequence of the mRNA for a gene and/or determining the amino acid sequence encoded thereby, and comparing these sequences to a second nucleotide or amino acid sequence. Genomic sequences can also be determined and compared in this fashion. In general, identity refers to an exact nucleotide-to-nucleotide or amino acid-to-amino acid correspondence of two polynucleotides or polypeptide sequences, respectively. Two or more sequences (polynucleotide or amino acid) can be compared by determining their percent identity. The percent identity of two sequences, whether nucleic acid or amino acid sequences, is the number of exact matches between two aligned sequences divided by the length of the shorter sequences and multiplied by 100. An approximate alignment for nucleic acid sequences is provided by the local homology algorithm of Smith and Waterman, Advances in Applied Mathematics 2:482-489 (1981). This algorithm can be applied to amino acid sequences by using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov, Nucl. Acids Res. 14(6):6745-6763 (1986). An exemplary implementation of this algorithm to determine percent identity of a sequence is provided by the Genetics Computer Group (Madison, Wis.) in the “BestFit” utility application. Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations-FSwiss protein+Spupdate+PIR. Details of these programs can be found on the GenBank website. With respect to sequences described herein, the range of desired degrees of sequence identity is approximately 80% to 100% and any integer value therebetween. Typically the percent identities between sequences are at least 70-75%, preferably 80-82%, more preferably 85-90%, even more preferably 92%, still more preferably 95%, and most preferably 98% sequence identity.

Alternatively, the degree of sequence similarity between polynucleotides can be determined by hybridization of polynucleotides under conditions that allow formation of stable duplexes between regions that share a degree of sequence identity, followed by digestion with single-stranded-specific nuclease(s), and size determination of the digested fragments. Two nucleic acid, or two polypeptide sequences are substantially similar to each other when the sequences exhibit at least about 70%-75%, preferably 80%-82%, more-preferably 85%-90%, even more preferably 92%, still more preferably 95%, and most preferably 98% sequence identity over a defined length of the molecules, as determined using the methods above. As used herein, substantially similar also refers to sequences showing complete identity to a specified DNA or polypeptide sequence. DNA sequences that are substantially similar can be identified in a Southern hybridization experiment under, for example, stringent conditions, as defined for that particular system. Defining appropriate hybridization conditions is within the skill of the art. See, e.g., Sambrook et al., supra; Nucleic Acid Hybridization: A Practical Approach, editors B. D. Hames and S. J. Higgins, (1985) Oxford; Washington, D.C.; IRL Press).

Selective hybridization of two nucleic acid fragments can be determined as follows. The degree of sequence identity between two nucleic acid molecules affects the efficiency and strength of hybridization events between such molecules. A partially identical nucleic acid sequence will at least partially inhibit the hybridization of a completely identical sequence to a target molecule. Inhibition of hybridization of the completely identical sequence can be assessed using hybridization assays that are well known in the art (e.g., Southern (DNA) blot, Northern (RNA) blot, solution hybridization, or the like, see Sambrook, et al., Molecular Cloning: A Laboratory Manual, Second Edition, (1989) Cold Spring Harbor, N.Y.). Such assays can be conducted using varying degrees of selectivity, for example, using conditions varying from low to high stringency. If conditions of low stringency are employed, the absence of non-specific binding can be assessed using a secondary probe that lacks even a partial degree of sequence identity (for example, a probe having less than about 30% sequence identity with the target molecule), such that, in the absence of non-specific binding events, the secondary probe will not hybridize to the target.

When utilizing a hybridization-based detection system, a nucleic acid probe is chosen that is complementary to a reference nucleic acid sequence, and then by selection of appropriate conditions the probe and the reference sequence selectively hybridize, or bind, to each other to form a duplex molecule. A nucleic acid molecule that is capable of hybridizing selectively to a reference sequence under moderately stringent hybridization conditions typically hybridizes under conditions that allow detection of a target nucleic acid sequence of at least about 10-14 nucleotides in length having at least approximately 70% sequence identity with the sequence of the selected nucleic acid probe. Stringent hybridization conditions typically allow detection of target nucleic acid sequences of at least about 10-14 nucleotides in length having a sequence identity of greater than about 90-95% with the sequence of the selected nucleic acid probe. Hybridization conditions useful for probe/reference sequence hybridization, where the probe and reference sequence have a specific degree of sequence identity, can be determined as is known in the art (see, for example, Nucleic Acid Hybridization: A Practical Approach, editors B. D. Hames and S. J. Higgins, (1985) Oxford; Washington, D.C.; IRL Press). Conditions for hybridization are well-known to those of skill in the art.

Hybridization stringency refers to the degree to which hybridization conditions disfavor the formation of hybrids containing mismatched nucleotides, with higher stringency correlated with a lower tolerance for mismatched hybrids. Factors that affect the stringency of hybridization are well-known to those of skill in the art and include, but are not limited to, temperature, pH, ionic strength, and concentration of organic solvents such as, for example, formamide and dimethylsulfoxide. As is known to those of skill in the art, hybridization stringency is increased by higher temperatures, lower ionic strength and lower solvent concentrations. With respect to stringency conditions for hybridization, it is well known in the art that numerous equivalent conditions can be employed to establish a particular stringency by varying, for example, the following factors: the length and nature of the sequences, base composition of the various sequences, concentrations of salts and other hybridization solution components, the presence or absence of blocking agents in the hybridization solutions (e.g., dextran sulfate, and polyethylene glycol), hybridization reaction temperature and time parameters, as well as, varying wash conditions. A particular set of hybridization conditions may be selected following standard methods in the art (see, for example, Sambrook, et al., Molecular Cloning: A Laboratory Manual, Second Edition, (1989) Cold Spring Harbor, N.Y.).

### EXAMPLES

The following examples are included to illustrate the invention.

### Example 1

### Genome Editing of NOG locus

Zinc finger nucleases (ZFNs) that target and cleave the NOG locus of rats may be designed, assembled and validated using strategies and procedures previously described (see Geurts et al. Science (2009) 325:433). ZFN design may make use of an archive of pre-validated 1-finger and 2-finger modules. The rat NOG gene region was scanned for putative zinc finger binding sites to which existing modules could be fused to generate a pair of 4-, 5-, or 6-finger proteins that would bind a 12-18 bp sequence on one strand and a 12-18 bp sequence on the other strand, with about 5-6 bp between the two binding sites.

Capped, polyadenylated mRNA encoding pairs of ZFNs may be produced using known molecular biology techniques. The mRNA may be transfected into rat cells. Control cells may be injected with mRNA encoding GFP. Active ZFN pairs may be identified by detecting ZFN-induced double strand chromosomal breaks using the Cel-1 nuclease assay. This assay detects alleles of the target locus that deviate from wild type (WT) as a result of non-homologous end joining (NHEJ)-mediated imperfect repair of ZFN-induced DNA double strand breaks. PCR amplification of the targeted region from a pool of ZFN-treated cells generates a mixture of WT and mutant amplicons. Melting and reannealing of this mixture results in mismatches forming between heteroduplexes of the WT and mutant alleles. A DNA “bubble” formed at the site of mismatch is cleaved by the surveyor nuclease Cel-1, and the cleavage products can be resolved by gel electrophoresis. This assay may be used to identify a pair of active ZFNs that edited the APP locus.

To mediate editing of the NOG gene locus in animals, fertilized rat embryos may be microinjected with mRNA encoding the active pair of ZFNs using standard procedures (e.g., see Geurts et al. (2009) supra). The injected embryos may be either incubated in vitro, or transferred to pseudopregnant female rats to be carried to parturition. The resulting embryos/fetus, or the toe/tail clip of live born animals may be harvested for DNA extraction and analysis. DNA may be isolated using standard procedures. The targeted region of the NOG locus may be PCR amplified using appropriate primers. The amplified DNA may be subcloned into a suitable vector and sequenced using standard methods.

### Example 2

### Genome Editing of BMP4 in a Model Organism

ZFN-mediated genome editing may be used to study the effects of a “knockout” mutation in neurodevelopmental chromosomal sequence, such as a chromosomal sequence encoding the BMP4 protein, in a genetically modified model animal and cells derived from the animal. Such a model animal may be a rat. In general, ZFNs that bind to the rat chromosomal sequence encoding the BMP4 protein associated with a neurodevelopmental pathway may be used to introduce a deletion or insertion such that the coding region of the BMP4 gene is disrupted such that a functional BMP4 protein may not be produced.

Suitable fertilized embryos may be microinjected with capped, polyadenylated mRNA encoding the ZFN essentially as detailed above in Example 1. The frequency of ZFN-induced double strand chromosomal breaks may be determined using the Cel-1 nuclease assay, as detailed above. The sequence of the edited chromosomal sequence may be analyzed as described above. The development of the neurodevelopmental symptoms and disorders caused by the BMP4 “knockout” may be assessed in the genetically modified rat or progeny thereof. Furthermore, molecular analyses of neurodevelopmental pathways may be performed in cells derived from the genetically modified animal comprising a BMP4 “knockout”.

### Example 3

### Generation of a Humanized Rat Expressing a Mutant Form of Human BMP4

Four missense mutations in BMP4 were detected in a population of human spina bifida aperta patients. ZFN-mediated genome editing may be used to generate a humanized rat wherein the rat BMP4 gene is replaced with a mutant form of the human BMP4 gene associated with spina bifida aperta, or any combination of the four mutations. Such a humanized rat may be used to study the development of the spina bifida aperta associated with the mutant human BMP4 protein. In addition, the humanized rat may be used to assess the efficacy of potential therapeutic agents targeted at the pathway leading to spina bifida aperta comprising BMP4.

The genetically modified rat may be generated using the methods described in the Example 1. However, to generate the humanized rat, the ZFN mRNA may be co-injected with the human chromosomal sequence encoding the mutant BMP4 protein into the rat embryo. The rat chromosomal sequence may then be replaced by the mutant human sequence by homologous recombination, and a humanized rat expressing a mutant form of the BMP4 protein may be produced.

What is claimed is:

1. A genetically modified animal comprising at least one edited chromosomal sequence encoding a neurodevelopmental protein.

2. The genetically modified animal of claim 1, wherein the edited chromosomal sequence is inactivated, modified, or comprises an integrated sequence.

3. The genetically modified animal of claim 1, wherein the edited chromosomal sequence is inactivated such that no functional neurodevelopmental protein associated is produced.

4. The genetically modified animal of claim 3, wherein the inactivated chromosomal sequence comprises no exogenously introduced sequence.

5. The genetically modified animal of claim 3, further comprising at least one chromosomally integrated sequence encoding a functional neurodevelopmental protein.

6. The genetically modified animal of claim 1, wherein the neurodevelopmental protein is chosen from BMP4, CHRD, NOG, WNT2, WNT2B, WNT3A, WNT4, WNT5A, WNT6, WNT7B, WNT8B, WNT9A, WNT9B, WNT10A, WNT10B, WNT16, OTX2, GBX2, FGF8, RELN, DAB1, POU4F1, NUMB, and combinations thereof.

7. The genetically modified animal of claim 1, further comprising a conditional knock-out system for conditional expression of the neurodevelopmental protein.

8. The genetically modified animal of claim 1, wherein the edited chromosomal sequence comprises an integrated reporter sequence.

9. The genetically modified animal of claim 1, wherein the animal is heterozygous or homozygous for the at least one edited chromosomal sequence.

10. The genetically modified animal of claim 1, wherein the animal is an embryo, a juvenile, or an adult.

11. The genetically modified animal of claim 1, wherein the animal is chosen from bovine, canine, equine, feline, ovine, porcine, non-human primate, and rodent.

12. The genetically modified animal of claim 1, wherein the animal is rat.

13. The genetically modified animal of claim 4, wherein the animal is rat and the protein is an ortholog of a human neurodevelopmental protein.

14. A non-human embryo, the embryo comprising at least one RNA molecule encoding a zinc finger nuclease that recognizes a chromosomal sequence encoding a neurodevelopmental protein, and, optionally, at least one donor polynucleotide comprising a sequence encoding an ortholog of the neurodevelopmental protein or an edited neurodevelopmental protein.

15. The non-human embryo of claim 14, wherein the neurodevelopmental protein is chosen from BMP4, CHRD, NOG, WNT2, WNT2B, WNT3A, WNT4, WNT5A, WNT6, WNT7B, WNT8B, WNT9A, WNT9B, WNT10A, WNT10B, WNT16, OTX2, GBX2, FGF8, RELN, DAB1, POU4F1, NUMB, and combinations thereof.

16. The non-human embryo of claim 14, wherein the embryo is chosen from bovine, canine, equine, feline, ovine, porcine, non-human primate, and rodent.

17. The non-human embryo of claim 14, wherein the embryo is rat and the protein is an ortholog of a human neurodevelopmental protein.

18. A genetically modified cell, the cell comprising at least one edited chromosomal sequence encoding a neurodevelopmental protein.

19. The genetically modified cell of claim 18, wherein the edited chromosomal sequence is inactivated, modified, or comprises an integrated sequence.

20. The genetically modified cell of claim 19, wherein the edited chromosomal sequence is inactivated such that no functional neurodevelopmental protein is produced.

21. The genetically modified cell of claim 20, further comprising at least one chromosomally integrated sequence encoding a functional neurodevelopmental protein.

22. The genetically modified cell of claim 18, wherein the neurodevelopmental protein is chosen from BMP4, CHRD, NOG, WNT2, WNT2B, WNT3A, WNT4, WNT5A, WNT6, WNT7B, WNT8B, WNT9A, WNT9B, WNT10A, WNT10B, WNT16, OTX2, GBX2, FGF8, RELN, DAB1, POU4F1, NUMB, and combinations thereof.

23. The genetically modified cell of claim 18, wherein the cell is heterozygous or homozygous for the at least one edited chromosomal sequence.

24. The genetically modified cell of claim 18, wherein the cell is of bovine, canine, equine, feline, human, ovine, porcine, non-human primate, or rodent origin.

25. The genetically modified cell of claim 18, wherein the cell is of rat origin and the protein is an ortholog of a human neurodevelopmental protein.

26. A method for assessing the effect of an agent in a genetically modified animal, the method comprising administering the agent to the genetically modified animal comprising at least one edited chromosomal sequence encoding a neurodevelopmental protein, and comparing a parameter obtained from the genetically modified animal to the parameter obtained from a wild-type animal administered the same agent, wherein the parameter is chosen from:
a) rate of elimination of the agent or its metabolite(s);
b) circulatory levels of the agent or its metabolite(s);
c) bioavailability of the agent or its metabolite(s);
d) rate of metabolism of the agent or its metabolite(s);
e) rate of clearance of the agent or its metabolite(s);
f) toxicity of the agent or its metabolite(s); and
g) ability of the agent to modify an incidence or indication of a neurodevelopmental disorder in the genetically modified animal.

27. The method of claim 26, wherein the agent is a pharmaceutically active ingredient, a drug, a toxin, or a chemical.

28. The method of claim 26, wherein the at least one edited chromosomal sequence is inactivated such that no functional neurodevelopmental protein is produced, and wherein the genetically modified animal further comprises at least one chromosomally integrated sequence encoding an ortholog of the neurodevelopmental protein.

29. The method of claim 26, wherein the neurodevelopmental protein is chosen from BMP4, CHRD, NOG, WNT2, WNT2B, WNT3A, WNT4, WNT5A, WNT6, WNT7B, WNT8B, WNT9A, WNT9B, WNT10A, WNT10B, WNT16, OTX2, GBX2, FGF8, RELN, DAB1, POU4F1, NUMB, and combinations thereof.

30. The method of claim 26, wherein the animal is a rat of a strain chosen from Dahl Salt-Sensitive, Fischer 344, Lewis, Long Evans Hooded, Sprague-Dawley, and Wistar.

31. The method of claim 26, wherein the incidence or indication of the neurodevelopmental disorder occurs spontaneously in the genetically modified animal.

32. The method of claim 26, wherein the incidence or indication of the neurodevelopmental disorder is promoted by exposure to a neurodisruptive agent.

33. The method of claim 32, wherein the neurodisruptive agent is chosen from a neurodevelopmental protein, a drug, a toxin, a chemical, and an environmental stress.

34. A method for assessing the therapeutic potential of an agent as a treatment for a neurodevelopmental disorder, the method comprising administering the agent to a genetically modified animal, wherein the genetically modified animal comprises at least one edited chromosomal sequence encoding a neurodevelopmental protein, and comparing a selected parameter obtained from the genetically modified animal to the selected parameter obtained from a wild-type animal with no exposure to the same agent, wherein the selected parameter is chosen from:
a) spontaneous behaviors;
b) performance during behavioral testing;
c) physiological anomalies;
d) abnormalities in tissues or cells;
e) biochemical function; and
f) molecular structures.

35. The method of claim 34, wherein the agent comprises at least one pharmaceutically active compound.

36. The method of claim 34, wherein the at least one edited chromosomal sequence is inactivated such that no functional neurodevelopmental protein is produced, and wherein the animal further comprises at least one chromosomally integrated sequence encoding an ortholog of the neurodevelopmental protein.

37. The method of claim 34, wherein the neurodevelopmental protein is chosen from BMP4, CHRD, NOG, WNT2, WNT2B, WNT3A, WNT4, WNT5A, WNT6, WNT7B, WNT8B, WNT9A, WNT9B, WNT10A, WNT10B, WNT16, OTX2, GBX2, FGF8, RELN, DAB1, POU4F1, NUMB, and combinations thereof.

38. The method of claim 34, wherein the animal is a rat of a strain chosen from Dahl Salt-Sensitive, Fischer 344, Lewis, Long Evans Hooded, Sprague-Dawley, and Wistar.

39. The method of claim 34, wherein the incidence or indication of the neurodevelopmental disorder occurs spontaneously in the genetically modified animal.

40. The method of claim 34, wherein the incidence or indication of the neurodevelopmental disorder is promoted by exposure to a neurodisruptive agent.

41. The method of claim 40, wherein the neurodisruptive agent is chosen from a neurodevelopmental protein, a drug, a toxin, a chemical, and an environmental stress.
